SEQUENCE LISTING (43 sequences)

SEQ ID NO: 1 - protein, 738 aa, Homo sapiens
  CD31
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 2 - protein, 300 aa, Homo sapiens
  CD38
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 3 - protein, 585 aa, Mus musculus
  Soluble CD31 (sCD31)
MLLALGLTLVLYASLQAEENSFTINSIHMESLPSWEVMNGQQLTLECLVDISTTSKSRSQHRVLFYKDDAMVYNVTSREHTESYVIPQARVFHSGKYKCTVMLNNKEKTTIEYEVKVHGVSKPKVTLDKKEVTEGGVVTVNCSLQEEKPPIFFKIEKLEVGTKFVKRRIDKTSNENFVLMEFPIEAQDHVLVFRCQAGILSGFKLQESEPIRSEYVTVQESFSTPKFEIKPPGMIIEGDQLHIRCIVQVTHLVQEFTEIIIQKDKAIVATSKQSSEAVYSVMAMVEYSGHYTCKVESNRISKASSIMVNITELFPKPKLEFSSSRLDQGELLDLSCSVSGTPVANFTIQKEETVLSQYQNFSKIAEESDSGEYSCTAGIGKVVKRSGLVPIQVCEMLSKPSIFHDAKSEIIKGHAIGISCQSENGTAPITYHLMKAKSDFQTLEVTSNDPATFTDKPTRDMEYQCRADNCHSHPAVFSEILRVRVIAPVDEVVISILSSNEVQSGSEMVLRCSVKEGTSPITFQFYKEKEDRPFHQAVVNDTQAFWHNKQASKKQEGQYYCTASNRASSMRTSPRSSTLAVRVFL

SEQ ID NO: 4 - protein, 7 aa, Artificial Sequence
  VH-CDR1 Consensus
  SITE 7: X is selected from Tyr (Y), Asn (N) and Ser (S)
GFTFSNX

SEQ ID NO: 5 - protein, 6 aa, Artificial Sequence
  SITE 1: X is selected from Ser (S) and Tyr (Y)
  VH-CDR2 Consensus
  SITE 6: X is selected from Tyr (Y), Asp (D), Asn (N) and Ser (S)
XGSSRX

SEQ ID NO: 6 - protein, 14 aa, Artificial Sequence
  SITE 1: X is selected from Ser (S) and an empty position
  VH-CDR3 Consensus
  SITE 2: X is selected from Ser (S) and an empty position
  SITE 3: X is selected from Ser (S) and Tyr (Y)
  SITE 4: X is selected from Ser (S) and Asp (D)
  SITE 5: X is selected from Tyr (Y), Ser (S), Asp (D) and Gly (G)
  SITE 7: X being selected from Tyr (Y) and Gly (G)
  SITE 8: X being selected from Ser (S), Tyr (Y) and Phe (F)
  SITE 9: X being selected from Gly (G) and Asp (D)
  SITE 10: X being selected from Asn (N), Tyr (Y) and Ser (S)
XXXXXYXXXXGMDV

SEQ ID NO: 7 - protein, 7 aa, Artificial Sequence
  VH-CDR1
GFTFSNY

SEQ ID NO: 8 - protein, 7 aa, Artificial Sequence
  VH-CDR1
GFTFSNN

SEQ ID NO: 9 - protein, 7 aa, Artificial Sequence
  VH-CDR1
GFTFSNS

SEQ ID NO: 10 - protein, 6 aa, Artificial Sequence
  VH-CDR2
SGSSRS

SEQ ID NO: 11 - protein, 6 aa, Artificial Sequence
  VH-CDR2
SGSSRY

SEQ ID NO: 12 - protein, 6 aa, Artificial Sequence
  VH-CDR2
SGSSRN

SEQ ID NO: 13 - protein, 6 aa, Artificial Sequence
  VH-CDR2
YGSSRD

SEQ ID NO: 14 - protein, 6 aa, Artificial Sequence
  VH-CDR2
SGSSRD

SEQ ID NO: 15 - protein, 13 aa, Artificial Sequence
  VH-CDR3
SSDDYYYDYGMDV

SEQ ID NO: 16 - protein, 14 aa, Artificial Sequence
  VH-CDR3
SSSSYYYSGNGMDV

SEQ ID NO: 17 - protein, 14 aa, Artificial Sequence
  VH-CDR3
SSYSSYGSGNGMDV

SEQ ID NO: 18 - skipped

SEQ ID NO: 19 - protein, 12 aa, Artificial Sequence
  VH-CDR3
SSGYYFGYGMDV

SEQ ID NO: 20 - protein, 14 aa, Artificial Sequence
  VH-CDR3
SSSSYYYSGSGMDV

SEQ ID NO: 21 - protein, 14 aa, Artificial Sequence
  VL-CDR1 Consensus
  SITE 10: X being selected from Ser (S) and Asn (N)
  SITE 11: X being selected from Ser (S) and Tyr (Y)
  SITE 12: X being selected from Tyr (Y), and Ser (S)
AGTSSDVGGXXXVS

SEQ ID NO: 22 - protein, 7 aa, Artificial Sequence
  SITE 1: X being selected from Tyr (Y), Ser (S) and Asp (D)
  VL-CDR2 Consensus
  SITE 4: X being selected from Tyr (Y) and Asn (N)
XDSXRPS

SEQ ID NO: 23 - protein, 9 aa, Artificial Sequence
  VL-CDR3 Consensus
STRVFGGGT

SEQ ID NO: 24 - protein, 14 aa, Artificial Sequence
  VL-CDR1
AGTSSDVGGSYSVS

SEQ ID NO: 25 - protein, 14 aa, Artificial Sequence
  VL-CDR1
AGTSSDVGGSSYVS

SEQ ID NO: 26 - skipped

SEQ ID NO: 27 - protein, 14 aa, Artificial Sequence
  VL-CDR1
AGTSSDVGGSYYVS

SEQ ID NO: 28 - protein, 14 aa, Artificial Sequence
  VL-CDR1
AGTSSDVGGNSYVS

SEQ ID NO: 29 - protein, 7 aa, Artificial Sequence
  VL-CDR2
DDSNRPS

SEQ ID NO: 30 - protein, 7 aa, Artificial Sequence
  VL-CDR2
YDSYRPS

SEQ ID NO: 31 - skipped

SEQ ID NO: 32 - protein, 7 aa, Artificial Sequence
  VL-CDR2
SDSYRPS

SEQ ID NO: 33 - protein, 122 aa, Artificial Sequence
  HCVR B08
EVQLVESGGSLVKPGGSLRLSCAASGFTFSNYYMNWVRQAPGKGLEWISSISGSSRSIYYADFVKGRFTISRDNAKNSLYLQMNSLRAEDTAVYYCVRSSDDYYYDYGMDVWGRGTLVTVSS

SEQ ID NO: 34 - protein, 123 aa, Artificial Sequence
  HCVR C05
EVQLVESGGSLVKPGGSLRLSCAASGFTFSNNYMNWVRQAPGKGLEWISSISGSSRYISYADFVKGRFTISRDNAKNSLYLQMNSLRAEDTAVYYCVRSSSSYYYSGNGMDVWGRGTLVTVSS

SEQ ID NO: 35 - protein, 123 aa, Artificial Sequence
  HCVR C06
EVQLVESGGSLVKPGGSLRLSCAASGFTFSNSAMNWVRQAPGKGLEWISSISGSSRNIYYADFVKGRFTISRDNAKNSLYLQMNSLRAEDTAVYYCVRSSYSSYGSGNGMDVWGRGTLVTVSS

SEQ ID NO: 36 - protein, 121 aa, Artificial Sequence
  HCVR D06
EVQLVESGGSLVKPGGSLRLSCAASGFTFSNYDMNWVRQAPGKGLEWISYIYGSSRDISYADFVKGRFTISRDNAKNSLYLQMNSLRAEDTAVYYCVRSSGYYFGYGMDVWGRGTLVTVSS

SEQ ID NO: 37 - protein, 123 aa, Artificial Sequence
  HCVR D07
EVQLVESGGSLVKPGGSLRLSCAASGFTFSNSAMNWVRQAPGKGLEWISYISGSSRDISYADFVKGRFTISRDNAKNSLYLQMNSLRAEDTAVYYCVRSSSSYYYSGSGMDVWGRGTLVTVSS

SEQ ID NO: 38 - protein, 123 aa, Artificial Sequence
  HCVR D10
EVQLVESGGSLVKPGGSLRLSCAASGFTFSNSAMNWVRQAPGKGLEWISSISGSSRDIYYADFVKGRFTISRDNAKNSLYLQMNSLRAEDTAVYYCVRSSSSYYYSGNGMDVWGRGTLVTVSS

SEQ ID NO: 39 - protein, 107 aa, Artificial Sequence
  LCVR B08
QSVLTQPASVSGSPGQSITISCAGTSSDVGGSYSVSWYQQHPGKAPKLMIYDDSNRPSGVSNRFSGSKSGNTASLTISGLQAEDEADYYCSSYTDYSTRVFGGGTKL

SEQ ID NO: 40 - protein, 107 aa, Artificial Sequence
  LCVR C05/C06
QSVLTQPASVSGSPGQSITISCAGTSSDVGGSSYVSWYQQHPGKAPKLMIYYDSYRPSGVSNRFSGSKSGNTASLTISGLQAEDEADYYCSSNTYYSTRVFGGGTKL

SEQ ID NO: 41 - protein, 107 aa, Artificial Sequence
  LCVR D06
QSVLTQPASVSGSPGQSITISCAGTSSDVGGSYYVSWYQQHPGKAPKLMIYSDSYRPSGVSNRFSGSKSGNTASLTISGLQAEDEADYYCSSNTAYSTRVFGGGTKL

SEQ ID NO: 42 - protein, 107 aa, Artificial Sequence
  LCVR D07/D10
QSVLTQPASVSGSPGQSITISCAGTSSDVGGNSYVSWYQQHPGKAPKLMIYYDSYRPSGVSNRFSGSKSGNTASLTISGLQAEDEADYYCSSNTYYSTRVFGGGTKL

SEQ ID NO: 43 - protein, 609 aa, Homo sapiens [sequence omitted]

(19) United States
(12) Patent Application Publication
(10) Pub. No.: US 2022/0089765 A1
(43) Pub. Date: Mar. 24, 2022

(54) CD31 COMPETITORS AND USES THEREOF

(71) Applicant: ENCEFA, Saint-Maur-des-Fossés (FR)

(72) Inventors: Laurence BRESSAC, Saint-Maur-des-Fossés (FR); Serge GUERREIRO, Alfortville (FR); Damien TOULORGE, Paris (FR)

(73) Assignee: ENCEFA, Saint-Maur-des-Fossés (FR)

(21) Appl. No.: 17/425,022

(22) PCT Filed: Jan. 23, 2020
(86) PCT No.: PCT/EP2020/051667

§ 371 Date: Jul. 22, 2021

(30) Foreign Application Priority Data
Jan. 23, 2019 (EP) 19305086.1

(51) Int. Cl. C07K 16/28; A61K 38/17; A61P 25/16

(52) U.S. Cl. CPC C07K 16/2896; A61K 38/1774; C07K 2317/565; C07K 2317/24; A61P 25/16

(57) ABSTRACT

Isolated anti-human CD38 antibodies or antigen-binding fragment thereof; nucleic acids and expression vectors encoding the same. Also, compounds which specifically compete with CD31 for CD38 binding, for use in preventing and/or treating a disease selected from neurodegenerative diseases, neuroinflammatory diseases, inflammatory diseases, autoimmune diseases, metabolic diseases, ocular diseases, age-related diseases, cancer and metastasis in a subject in need thereof.

### FIELD OF THE INVENTION

The present invention relates to the field of prevention and/or treatment of diseases.

In particular, the invention relates to a compound, useful for the prevention and/or treatment of a disease, said compound being a competitor of CD31.

### BACKGROUND

CD31, also known as Platelet endothelial cell adhesion molecule-1 (PECAM-1), is a 130 kDa type I transmembrane glycoprotein that consists of six extracellular immunoglobulins (Ig)-like homology domains, a 19-residue transmembrane domain, and a 118-residue cytoplasmic tail (Newman and Newman, 2003. Arterioscler Thromb Vasc Biol. 23:953-964). As such, CD31 belongs to the Ig superfamily of cell adhesion molecules. CD31 is able to bind to itself through homophilic interaction involving Ig-like domain 1; as well to two other ligands, αVβ3 integrin and CD38, through heterophilic interaction involving Ig-like domains 1-3 (Newman, 1997. J Clin Invest. 99(1):3-8).

CD31 expression is mainly observed in endothelial cells, where it is considered as a constitutive marker (Kalinowska & Losy, 2006. Eur J Neurol. 13(12):1284-90), but also in most non-erythroid cells of the hematopoietic lineage including platelets, monocytes, neutrophils, T and B cell subsets (Wang et al., 2003. Am J Physiol Heart Circ Physiol. 284(3):H1008-17). While CD31 homophilic interaction is a major constituent of the endothelial cell intercellular junction, it is also involved in the process of leukocyte-endothelial transmigration (diapedesis), allowing the penetration of leucocytes into tissue during inflammation (Ilan & Madri, 2003. Curr Opin Cell Biol. 15(5):515-24).

CD31 is found under both a membrane form and a soluble form, following metalloproteinase-dependent cleavage (Ilan et al., 2001. FASEB J. 15(2):362-72). Increased circulating levels of soluble CD31 (sCD31) were observed in some inflammatory diseases, including atherosclerosis and sepsis (Feng et al., 2016. Eur Rev Med Pharmacol Sci. 20(19):4082-4088; Kjaergaard et al., 2016. APMIS. 124(10): 846-55), which might reflect the fact that sCD31 is involved in reducing further leukocyte transmigration in a negative feedback loop pattern (Muller et al., 1993. J Exp Med. 178(2):449-60). Of interest, increased cerebrospinal fluid (CSF) sCD31 levels were also observed in several neurodegenerative diseases including multiple sclerosis (Losy et al., 1999. J Neuroimmunol. 99(2): 169-72, Kuenz et al., 2005. J Neuroimmunol. 167(1-2): 143-9), HIV-encephalitis (Eugenin et al., 2006. J Leukoc Biol. 79(3):444-52), paraneoplastic encephalo-myelo-polyneuropathy (Dziewulska et al., 2000. Folia Neuropathol. 38(1):29-33), as well as cerebral ischemia (Zaremba and Losy, 2002. Acta Neurol Scand. 106(5):292-8). The general understanding of these increased brain sCD31 levels is that it indicates disruption of the blood-brain barrier (BBB), which is typical for any neuro-inflammatory reaction (Kalinowska and Losy, 2006. Eur J Neurol. 13(12):1284-90).

However, brain sCD31 levels after ischemic stroke were found to correlate positively with (i) neurological stroke severity and (ii) with the degree of functional disability (Zaremba and Losy, 2002. Central-European J Immunol. 27:90-96). Thus, the Applicant asked whether sCD31 was actively engaged in the neurodegenerative process. Indeed, sCD31 could also be toxic by itself to neurons. Unexpectedly, the Applicant found that sCD31 was not toxic, but quite to the contrary, strongly protected neurons in vitro. This result is particularly surprising since CD31 expression in the brain is restricted to endothelial cells of the BBB (Williams et al., 1996. J Neurosci Res. 45(6):747-57). The neuroprotective effect of sCD31 was antagonized in the presence of the neutralizing clone Moon-1 anti-CD31 antibody, as well as by antagonistic anti-CD38 antibodies (clone OKT10 or AT13/5), demonstrating that the neuroprotective effect of sCD31 is mediated through interaction with its ligand CD38. Moreover, the neuroprotective effect of sCD31 was recapitulated by agonistic anti-CD38 antibodies that were previously shown to mimic the interaction between sCD31 and CD38 (Deaglio et al., 1998. J Immunol. 160(1):

395-402). Of note, the neuroprotective effect of agonistic anti-CD38 antibody (clone HB7) was, like that of sCD31, inhibited in the presence of antagonistic anti-CD38 antibodies (clone OKT10 or AT13/5).

CD38 is a 45 kDa type II transmembrane glycoprotein with a long C-terminal extracellular domain and a short N-terminal cytoplasmic domain. CD38 have several biological activities, including a receptor-mediated function through internalization, a tyrosine phosphorylation-mediated function and an enzyme-mediated function. In addition, some specific anti-CD38 antibodies were reported to be of interest in the treatment of cancer, as disclosed in WO2018224683. The Applicant found, herein, that the neuroprotective effect of sCD31 or agonistic anti-CD38 antibody (clone HB7) was antagonized when tyrosine phosphorylation was inhibited.

### SUMMARY

The present invention relates to an isolated anti-human CD38 antibody or antigen-binding fragment thereof, wherein said antibody or antigen-binding fragment thereof competes with CD31, preferably with human CD31; and induces lysosomal exocytosis in a cell, that is inhibited in the presence of vacuolin-1.

In one embodiment, said isolated anti-human CD38 antibody or antigen-binding fragment thereof is monoclonal.

In one embodiment, said isolated anti-human CD38 antibody or antigen-binding fragment thereof is humanized.

In one embodiment,
a) the variable region of the heavy chain (HCVR) of said isolated anti-human CD38 antibody or antigen-binding fragment thereof comprises the three following complementary-determining regions (CDRs):

(SEQ ID NO: 4)
V_{H}-CDR1: GFTFSNX₁,
(SEQ ID NO: 5)
V_{H}-CDR2: X₂GSSRX₃, and
(SEQ ID NO: 6)
V_{H}-CDR3: X₄X₅X₆X₇X8YX₉X₁₀X₁₁X₁₂GMDV;

and
b) the variable region of the light chain (LCVR) of said isolated anti-human CD38 antibody or antigen-binding fragment thereof comprises the three following CDRs:

(SEQ ID NO: 21)
V_{L}-CDR1: AGTSSDVGGX₁₃X₁₄X₁₅VS,
(SEQ ID NO: 22)
V_{L}-CDR2: X₁₆DSX₁₇RPS, and
(SEQ ID NO: 23)
V_{L}-CDR3: STRVFGGGT;

wherein:
   X₁ is selected from Tyr (Y), Asn (N) and Ser (S);
   X₂ is selected from Ser (S) and Tyr (Y);
   X₃ is selected from Tyr (Y), Asp (D), Asn (N) and Ser (S);
   X₄ is selected from Ser (S) and an empty position;
   X₅ is selected from Ser (S) and an empty position;
   X₆ is selected from Ser (S) and Tyr (Y);
   X₇ is selected from Ser (S) and Asp (D);
   X₈ is selected from Tyr (Y), Ser (S), Asp (D) and Gly (G);
   X₉ being selected from Tyr (Y) and Gly (G);
   X₁₀ being selected from Ser (S), Tyr (Y) and Phe (F);
   X₁₁ being selected from Gly (G) and Asp (D);
   X₁₂ being selected from Asn (N), Tyr (Y) and Ser (S);
   X₁₃ being selected from Ser (S) and Asn (N);
   X₁₄ being selected from Ser (S) and Tyr (Y);
   X₁₅ being selected from Tyr (Y), and Ser (S);
   X₁₆ being selected from Tyr (Y), Ser (S) and Asp (D); and
   X₁₇ being selected from Tyr (Y) and Asn (N).

In one embodiment, said anti-human CD38 antibody or antigen-binding fragment thereof comprises:
a V_{H}-CDR1 with SEQ ID NO: 7, a V_{H}-CDR2 with SEQ ID NO: 10, a V_{H}-CDR3 with SEQ ID NO: 15, a V_{L}-CDR1 with SEQ ID NO: 24, a V_{L}-CDR2 with SEQ ID NO: 29 and a V_{L}-CDR3 with SEQ ID NO: 23;
a V_{H}-CDR1 with SEQ ID NO: 8, a V_{H}-CDR2 with SEQ ID NO: 11, a V_{H}-CDR3 with SEQ ID NO: 16, a V_{L}-CDR1 with SEQ ID NO: 25, a V_{L}-CDR2 with SEQ ID NO: 30 and a V_{L}-CDR3 with SEQ ID NO: 23;
a V_{H}-CDR1 with SEQ ID NO: 9, a V_{H}-CDR2 with SEQ ID NO: 12, a V_{H}-CDR3 with SEQ ID NO: 17, a V_{L}-CDR1 with SEQ ID NO: 25, a V_{L}-CDR2 with SEQ ID NO: 30 and a V_{L}-CDR3 with SEQ ID NO: 23;
a V_{H}-CDR1 with SEQ ID NO: 7, a V_{H}-CDR2 with SEQ ID NO: 13, a V_{H}-CDR3 with SEQ ID NO: 19, a V_{L}-CDR1 with SEQ ID NO: 27, a V_{L}-CDR2 with SEQ ID NO: 32 and a V_{L}-CDR3 with SEQ ID NO: 23;
a V_{H}-CDR1 with SEQ ID NO: 9, a V_{H}-CDR2 with SEQ ID NO: 14, a V_{H}-CDR3 with SEQ ID NO: 20, a V_{L}-CDR1 with SEQ ID NO: 28, a V_{L}-CDR2 with SEQ ID NO: 30 and a V_{L}-CDR3 with SEQ ID NO: 23; or
a V_{H}-CDR1 with SEQ ID NO: 9, a V_{H}-CDR2 with SEQ ID NO: 14, a V_{H}-CDR3 with SEQ ID NO: 16, a V_{L}-CDR1 with SEQ ID NO: 28, a V_{L}-CDR2 with SEQ ID NO: 30 and a V_{L}-CDR3 with SEQ ID NO: 23.

In one embodiment, said anti-human CD38 antibody or antigen-binding fragment thereof comprises:
a HCVR with SEQ ID NO: 33 and a LCVR with SEQ ID NO: 39;
a HCVR with SEQ ID NO: 34 and a LCVR with SEQ ID NO: 40;
a HCVR with SEQ ID NO: 35 and a LCVR with SEQ ID NO: 40;
a HCVR with SEQ ID NO: 36 and a LCVR with SEQ ID NO: 41;
a HCVR with SEQ ID NO: 37 and a LCVR with SEQ ID NO: 42; or
a HCVR with SEQ ID NO: 38 and a LCVR with SEQ ID NO: 42.

The present invention also relates to a nucleic acid encoding the isolated anti-human CD38 antibody or antigen-binding fragment thereof according to the present invention.

The present invention also relates to an expression vector comprising the nucleic acid according to the present invention.

The present invention also relates to a compound which specifically competes with CD31 for CD38 binding, for use in preventing and/or treating a disease selected from neurodegenerative diseases; neuroinflammatory diseases; inflammatory diseases; autoimmune diseases; metabolic diseases; ocular diseases; age-related diseases; and cancer and metastasis; in a subject in need thereof.

In one embodiment, said disease is selected from amyotrophic lateral sclerosis; Parkinson's disease and related disorders; Alzheimer's disease and related disorders; Huntington disease; multiple sclerosis; rheumatoid arthritis; systemic lupus erythematosus; diabetes; obesity; non-alcoholic steatohepatitis; age-related macular degeneration; and glaucoma.

The present invention also relates to a compound which specifically competes with CD31 for CD38 binding, for use in increasing the level of at least one anti-inflammatory cytokine in a subject in need thereof, in particular in the blood of said subject.

In one embodiment, said anti-inflammatory cytokine is interleukin-10 (IL-10).

In one embodiment, the compound for use in any of the methods of the present invention is selected from a peptide, a chimeric peptide, an antibody, an antigen-binding fragment thereof, an antigen-binding antibody mimetic, an oligonucleotide and a small organic molecule; and said compound:
induces tyrosine phosphorylation of discrete cytoplasmic substrates in a cell, that is inhibited in the presence of genistein; and/or
induces lysosomal exocytosis in a cell, that is inhibited in the presence of vacuolin-1.

In one embodiment, the compound for use in any of the methods of the present invention is an isolated anti-human CD38 antibody or antigen-binding fragment thereof according to the present invention.

In one embodiment, the compound for use in any of the methods of the present invention is:
a peptide comprising the Ig-like domains 1-3 of human CD31, preferably a peptide comprising amino acids 28 to 315 of SEQ ID NO: 1 or a variant thereof; or
a chimeric peptide comprising the Ig-like domains 1-3 of human CD31, preferably a peptide comprising amino acids 28 to 315 of SEQ ID NO: 1 or a variant thereof; fused to a Fc domain of an immunoglobulin, to human serum albumin, preferably to the domain III of human serum albumin, or to transferrin.

### Definitions

Unless defined otherwise, all technical and scientific terms used herein have the same meaning as commonly understood to one skilled in the relevant art. For convenience, the meaning of certain terms and phrases employed in the specification, examples and claims are provided.

“Affitins”, as used herein, refers to artificial proteins with the ability to selectively bind antigens. They are structurally derived from the DNA binding protein Sac7d, found in Sulfolobus acidocaldarius, a microorganism belonging to the archaeal domain. By randomizing the amino acids on the binding surface of Sac7d, e.g., by generating variants corresponding to random substitutions of 11 residues of the binding interface of Sac7d, an affitin library may be generated and subjecting the resulting protein library to rounds of ribosome display, the affinity can be directed towards various targets, such as peptides, proteins, viruses and bacteria. Affitins are antibody mimetics and are being developed as tools in biotechnology. They have also been used as specific inhibitors for various enzymes (Krehenbrink et al., 2008. J Mol Biol. 383(5):1058-68). The skilled person may readily develop affitins with the required binding properties using methods know in the art, in particular as disclosed in International patent application WO2008068637 and the above-cited publication, in particular the generation of phage display and/or ribosome display libraries and their screening using an antigen as disclosed herein.

“Antibody” or “immunoglobulin”, as used herein, refer to a protein having a combination of two heavy and two light chains whether or not it possesses any relevant specific immunoreactivity. “Antibodies” refers to such assemblies which have significant known specific immunoreactive activity to an antigen of interest. The term encompasses polyclonal antibodies, monoclonal antibodies, recombinant antibodies, bispecific antibodies, multispecific antibodies and modified antibodies.

It will also be appreciated that the terms “antibody” and “immunoglobulin”, as used herein, encompass modified antibodies or antibody binding fragments, using known methods. For example, to slow clearance in vivo and obtain a more desirable pharmacokinetic profile, an antibody or binding fragment thereof may be modified with polyethylene glycol (PEG). Methods for coupling and site-specifically conjugating PEG to an antibody or binding fragment thereof are described in, e.g., Leong et al., 2001. Cytokine. 16(3):106-19; Delgado et al., 1996. Br J Cancer. 73(2):175-82.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (c) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (V_{L} domain) and heavy chain variable domain (V_{H} domain) of an antibody combine to form the variable region that defines a three-dimensional antigen binding site. This quaternary antibody structure forms the antigen binding site present at the end of each arm of the “Y”. More specifically, the antigen binding site is defined by three complementarity determining regions (CDRs) on each of the V_{H} and V_{L} domains.

Several researches to develop therapeutic antibodies have led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as N297A, N297G, L234A/L235A, L235E, T250Q/M428L, M252Y/S254T/T256E and H433K/N434F, have been shown to increase the binding affinity to FcRn and the half-life of IgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells or neurons, abrogating effector functions is required. Conversely, for antibodies intended for oncology use, increasing effector functions may improve the therapeutic activity. The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions. Binding of IgG to the FcγRs or C1q depends on residues located in the hinge region and the CH2 domain. Two regions of the CH2 domain are critical for FcγRs and C1q binding, and have unique sequences in IgG2 and IgG4.

“Antibody-dependent cell-mediated cytotoxicity” or “ADCC”, as used herein, refer to a form of cytotoxicity in which secreted antibodies bound onto Fc receptors (FcRs) present on certain cytotoxic cells (e.g., NK cells, neutrophils, monocytes and macrophages) enable these cytotoxic effector cells to bind specifically to an antigen-bearing target cell and subsequently to kill the target cell. To assess ADCC activity of a molecule of interest, an in vitro ADCC assay, such as that described in U.S. Pat. Nos. 5,500,362 or 5,821,337, may be performed.

“Antibody-dependent phagocytosis” or “opsonization” as used herein, refers to the cell-mediated reaction wherein non-specific cytotoxic cells that express FcγRs recognize bound antibody on a target cell and subsequently cause phagocytosis of the target cell.

“Anticalins”, as used herein, refer to artificial proteins that are able to bind to antigens, either to proteins or to small molecules. They are antibody mimetic derived from human lipocalins which are a family of naturally binding proteins. Anticalins are about eight times smaller with a size of about 180 amino acids and a mass of about 20 kDa (Kolmar & Skerra, 2008. FEBS J. 275(11):2667). Anticalin phage display libraries have been generated which allow for the screening and selection, in particular of anticalins with specific binding properties. The skilled person may readily develop anticalins with the required binding properties using methods known in the art, in particular as disclosed in EP patent EP1270725, US patent U.S. Pat. No. 8,536,307, Schlehuber & Skerra (2002. Biophys Chem. 96(2-3):213-28) and the above-cited publication, in particular the generation of phage display and/or ribosome display libraries and their screening using an antigen as disclosed herein.

“Antigen-binding antibody mimetic”, as used herein, refers to artificial proteins, peptides, nucleic acids, and more generally, any chemical compounds with the capacity to bind antigens mimicking that of antibodies.

Such mimetics include oligonucleotide aptamers, as well as affitins, anticalins and peptide aptamers. Affitins, anticalins and peptide aptamers may be produced in a number of expression system comprising bacterial expression systems or by combinatorial chemistry. Thus, the present invention provides affitins, anticalins, peptide aptamers and other similar antigen-binding antibody mimetics (such as oligonucleotide aptamers) with the features of the antibodies described herein, in particular with regard to the competition with CD31.

“Antigen-binding fragment of an antibody”, as used herein, refers to a part or region of an antibody, which comprises fewer amino acid residues than the whole antibody, i.e., a molecule corresponding to a portion of the structure of the antibody of the invention, that exhibits antigen-binding capacity for CD38, possibly in its native form; such fragment especially exhibits the same or substantially the same antigen-binding specificity for said antigen compared to the antigen-binding specificity of the corresponding whole antibody. Advantageously, the antigen-binding fragments have a similar binding affinity as the corresponding whole antibodies. However, antigen-binding fragments that have a reduced antigen-binding affinity with respect to corresponding whole antibodies are also encompassed within the invention. The antigen-binding capacity can be determined by measuring the affinity between the antibody and the target fragment. These antigen-binding fragments may also be designated as “functional fragments” of antibodies.

Antigen-binding fragments of an antibody encompass, without any limitation, Fv, dsFv, scFv, Fab, Fab′, F(ab′)2, and single-domain antibody. Fv fragments consist of the V_{L} and V_{H} domains of an antibody associated together by hydrophobic interactions; in dsFv fragments, the V_{H}:V_{L} heterodimer is stabilized by a disulfide bond; in scFv fragments, the V_{L} and V_{H} domains are connected to one another via a flexible peptide linker thus forming a single-chain protein. Fab fragments are monomeric fragments obtainable by papain digestion of an antibody; they comprise the entire L chain, and a V_{H}-C_{H}1 fragment of the H chain, bound together through a disulfide bond. The F(ab′)₂ fragment can be produced by pepsin digestion of an antibody below the hinge disulfide; it comprises two Fab′ fragments, and additionally a portion of the hinge region of the immunoglobulin molecule. The Fab′ fragments are obtainable from F(ab′)₂ fragments by cutting a disulfide bond in the hinge region. F(ab′)₂ fragments are divalent, i.e., they comprise two antigen binding sites, like the native immunoglobulin molecule; on the other hand, Fv (a V_{H}-V_{L} dimer constituting the variable part of Fab), dsFv, scFv, Fab, and Fab′ fragments are monovalent, i.e., they comprise a single antigen-binding site. These basic antigen-binding fragments of the invention can be combined together to obtain multivalent antigen-binding fragments, such as diabodies, tribodies or tetrabodies. These multivalent antigen-binding fragments are also part of the present invention.

“Bispecific antibodies”, as used herein, refers to antibodies that recognize two different antigens by virtue of possessing at least one region (e.g., derived from a variable region of a first antibody) that is specific for a first antigen, and at least a second region (e.g., derived from a variable region of a second antibody) that is specific for a second antigen. A bispecific antibody specifically binds to two target antigens and is thus one type of multispecific antibody. Multispecific antibodies, which recognize two or more different antigens, can be produced by recombinant DNA methods or include, but are not limited to, antibodies produced chemically by any convenient method. Bispecific antibodies include all antibodies or conjugates of antibodies, or polymeric forms of antibodies which are capable of recognizing two different antigens. Bispecific antibodies include antibodies that have been reduced and reformed so as to retain their bivalent characteristics and to antibodies that have been chemically coupled so that they can have several antigen recognition sites for each antigen such as BiME (Bispecific Macrophage Enhancing antibodies), BiTE (bispecific T cell engager), DART (Dual affinity retargeting); DNL (dock-and-lock), DVD-Ig (dual variable domain immunoglobulins), HAS (human serum albumin), kih (knobs into holes).

“CD31”, as used herein, refers to the 130 kDa type I transmembrane glycoprotein, also known as Platelet endothelial cell adhesion molecule-1 (PECAM-1), PECA1, GPIIA′, EndoCAM or CD31/EndoCAMas, and described in Newman & Newman (2003. Arterioscler Thromb Vasc Biol. 23:953-964). In the present invention, the term “CD31” refers more particularly to CD31 from a mammal species, even more particularly to human CD31. Preferably, the term “human CD31” refers to the protein of amino acid sequence SEQ ID NO: 1, referenced by the NP_000433 NCBI accession number. The numbering of amino acids of human CD31 as described herein corresponds to the numbering of amino acids of the human CD31 sequence set forth in SEQ ID NO: 1, and referenced by the NP_000433 NCBI accession number.

SEQ ID NO: 1
MQPRWAQGATMWLGVLLTLLLCSSLEGQENSFTINSVDMKSLPDWTVQN
GKNLTLQCFADVSTTSHVKPQHQMLFYKDDVLFYNISSMKSTESYFIPE
VRIYDSGTYKCTVIVNNKEKTTAEYQVLVEGVPSPRVTLDKKEAIQGGI
VRVNCSVPEEKAPIHFTIEKLELNEKMVKLKREKNSRDQNFVILEFPVE
EQDRVLSFRCQARIISGIHMQTSESTKSELVTVTESFSTPKFHISPTGM
IMEGAQLHIKCTIQVTHLAQEFPEIIIQKDKAIVAHNRHGNKAVYSVMA
MVEHSGNYTCKVESSRISKVSSIVVNITELFSKPELESSFTHLDQGERL
NLSCSIPGAPPANFTIQKEDTIVSQTQDFTKIASKSDSGTYICTAGIDK
VVKKSNTVQIVVCEMLSQPRISYDAQFEVIKGQTIEVRCESISGTLPIS
YQLLKTSKVLENSTKNSNDPAVFKDNPTEDVEYQCVADNCHSHAKMLSE
VLRVKVIAPVDEVQISILSSKVVESGEDIVLQCAVNEGSGPITYKFYRE
KEGKPFYQMTSNATQAFWTKQKASKEQEGEYYCTAFNRANHASSVPRSK
ILTVRVILAPWKKGLIAVVIIGVIIALLIIAAKCYFLRKAKAKQMPVEM
SRPAVPLLNSNNEKMSDPNMEANSHYGHNDDVRNHAMKPINDNKEPLNS
DVQYTEVQVSSAESHKDLGKKDTETVYSEVRKAVPDAVESRYSRTEGSL
DGT

“CD38”, as used herein, refers to the 45 kDa type II transmembrane glycoprotein also known as T10, cyclic ADP-ribose hydrolase 1, ADPRC1, as described in Malavasi et al. (2008. Physiological Review. 88:841-886). In the present invention, the term “CD38” refers more particularly to particularly from a mammal species, even more particularly to human CD38. Preferably, the term “human CD38” refers to the protein of amino acid sequence SEQ ID NO: 2, referenced by the NP_001766 NCBI accession number. The numbering of amino acids of human CD38 as described herein corresponds to the numbering of amino acids of the human CD38 sequence set forth in SEQ ID NO: 2, and referenced by the NP_001766 NCBI accession number.

SEQ ID NO: 2
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

“CDR” or “complementarity determining region”, as used herein, means the non-contiguous antigen combining sites found within the variable region of both heavy and light chain polypeptides. CDRs were identified according to the rules of Table 1 (as deduced from Kabat et al., 1991. Sequences of proteins of immunological interest (5^{th} ed.). Bethesda, Md.: U.S. Dep. of Health and Human Services; and Chothia and Lesk, 1987. J Mol Biol. 196(4):901-17), or by application of the IMGT “collier de perle” algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/−10%.

TABLE 1

| | Heavy chain variable region (HCVR or V_{H}) | | |
|---|---|---|---|
| | V_{H}-CDR1 | V_{H}-CDR2 | V_{H}-CDR3 |
| Start | Approx. at residue 26 | Always 15 residues after | Always 33 residues after |
| | (always 4 after a Cys) | the end of V_{H}-CDR1 | end of V_{H}-CDR2 |
| | according to | according to | Always 2 residues after a |
| | Chothia/AbM's definition | Kabat/AbM's definition | Cys |
| | Kabat's definition starts 5 | | |
| | residues later | | |
| Residue | Always Cys-Xaa-Xaa- | Typically, Leu-Glu-Trp- | Always Cys-Xaa-Xaa, |
| before | Xaa, with Xaa being any | Ile-Gly, but a number of | with Xaa being any |
| | amino acid according to | variations | amino acid |
| | Chothia/AbM's definition | | Typically, Cys-Ala-Arg |
| Residue | Always Trp | Lys/Arg- | Always Trp-Gly-Xaa- |
| after | Typically, Trp-Val, but | Leu/Ile/Val/Phe/Thr/Ala- | Gly, with Xaa being any |
| | also, Trp-Ile or Trp-Ala | Thr/Ser/Ile/Ala | amino acid |
| Length | 10 to 12 residues | 16 to 19 residues | 3 to 25 residues |
| | according to AbM's | according to Kabat's | |
| | definition | definition | |
| | Chothia's definition | AbM's definition ends 7 | |
| | excludes the last 4 | residues earlier | |
| | residues | | |
| | 5 to 7 residues according | | |
| | to Kabat's definition | | |
| | Light chain variable region (LCVR or V_{L}) | | |
| | V_{L}-CDR1 | V_{L}-CDR2 | V_{L}-CDR3 |
| Start | Approx. at residue 24 | Always 16 residues after | Always 33 residues after |
| | | the end of V_{L}-CDR1 | end of V_{L}-CDR2 (except |
| | | | NEW (PDB ID: 7FAB) |
| | | | which has the deletion at |
| | | | the end of CDR-L2*) |
| Residue | Always Cys | Generally, Ile-Tyr, but | Always Cys |
| before | | also, Val-Tyr, Ile-Lys or | |
| | | Ile-Phe | |
| Residue | Always Trp | | Always Phe-Gly-Xaa- |
| after | Typically, Trp-Tyr-Gln, | | Gly, with Xaa being any |
| | but also, Trp-Leu-Gln, | | amino acid |
| | Trp-Phe-Gln or Trp-Tyr- | | |
| | Leu | | |
| Length | 10 to 17 residues | Always 7 residues | 7 to 11 residues |
| | | (except NEW (PDB ID: | |
| | | 7FAB) which has a | |
| | | deletion in this region*) | |

*Saul & Poljak, 1992. Proteins. 14(3):363-71

“Chimeric antibody”, as used herein, refers to an antibody in which the sequence of the variable domain derived from the germline of a mammalian species, such as a mouse, have been grafted onto the sequence of the constant domain derived from the germline of another mammalian species, such as a human Advantageously, if the monoclonal antibody according to the invention is a chimeric monoclonal antibody, the latter comprises human constant regions. Starting from a non-human antibody, a chimeric antibody may be prepared by using genetic recombinant techniques well known to one skilled in the art. For example, the chimeric antibody may be produced by cloning for the heavy chain and the light chain a recombinant DNA including a promoter and a sequence coding for the variable region of the non-human antibody, and a sequence coding for the constant region of a human antibody. As for the methods for preparing chimeric antibodies, reference may be made, e.g., to Verhoeyn et al. (1988. Science. 239(4847):1534-6).

“Complement dependent cytotoxicity” or “CDC”, as used herein, refer to the lysis of a target cell in the presence of complement. Activation of the classical complement pathway is initiated by the binding of the first component of the complement system to antibodies which are bound to their cognate antigen. To assess complement activation, a CDC assay such as, e.g., the one described in Gazzano-Santoro et al., 1997. J Immunol Methods. 202(2):163-71, may be performed.

“Compound which specifically competes with CD31”, as used herein, relates to any molecule suitable for pharmaceutical uses, which specifically binds to CD38, thereby shunting endogenous CD31 interaction with CD38. Such compounds encompass antibodies, antigen-binding fragments thereof, antigen-binding antibody mimetics, small organic molecules, oligonucleotides and recombinant proteins.

“Consist(s/ing) essentially of”, with reference to a composition, pharmaceutical composition or medicament, is used herein to intend that the compound according to the invention is the only one agent with a biologic activity within said composition, pharmaceutical composition or medicament.

“Epitope”, as used herein, refers to a specific arrangement of amino acids located on a protein or proteins to which an antibody or binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Fragment crystallizable region” or “Fc” or “Fc region”, as used herein, encompass the polypeptides comprising the constant region of an antibody, excluding the first constant region immunoglobulin domain. Thus, Fc refers to the last two constant region immunoglobulin domains of IgA, IgD and IgG, and to the last three constant region immunoglobulin domains of IgE and IgM and the flexible hinge N-terminal to these domains.

For IgA and IgM, Fc may include the J chain. For IgG, Fc comprises immunoglobulin domains Cγ2 and Cγ3 and the hinge between Cγ1 and Cγ2.

Although the boundaries of the Fc region may vary, the human IgG heavy chain Fc region is usually defined to comprise residues C226 or P230 to its carboxyl-terminus, wherein the numbering is according to the EU index as set forth in Kabat et al., 1991 (Sequences of proteins of immunological interest (5^{th} ed.). Bethesda, Md.: U.S. Dep. of Health and Human Services). The EU index as set forth in Kabat refers to the residue numbering of the human IgG1 EU antibody as described in Kabat et al., 1991 (supra). Fc may refer to this region in isolation, or this region in the context of an antibody, antibody fragment, or Fc fusion protein.

The Fc fragment naturally consists of the constant region of the heavy chain excluding domain C_{H}1, i.e., of the lower boundary region and of the constant domains C_{H}2 and C_{H}3 or C_{H}2 to C_{H}4 (depending on the isotype).

In the sense of the invention, the Fc fragment of an antibody may be natural or may have been modified in various ways, provided that it comprises a functional domain for binding to FcR receptors (FcγR receptors for IgGs), and preferably a functional domain for binding to receptor FcRn. The modifications may include the deletion of certain portions of the Fc fragment, provided that the latter contains a functional domain for binding to receptors FcR (receptors FcγR for IgGs), and preferably a functional domain for binding to receptor FcRn. The modifications of the antibody or the Fc fragment of an antibody may also include various substitutions of amino acids able to affect the biological properties of the antibody, provided that the latter contains a functional domain for binding to receptors FcR, and preferably a functional domain for binding to receptor FcRn.

In particular, when the antibody is an IgG, it may comprise mutations intended to enhance the binding to receptor FcγRIII (CD16), as described in WO2000042072, WO2004029207, WO2004063351 or WO2004074455. Mutations permitting to enhance the binding to receptor FcRn, and therefore the half-life in vivo of the antibody, may also be present, as described, for example, in WO2000042072, WO2002060919, WO2010045193 or WO2010106180. Other mutations, such as those permitting to reduce or increase the binding to the proteins of the complement, and therefore the Complement Dependent Cytotoxicity (CDC) response, may be present or not, such as described, for example, in WO1999051642 or WO2004074455.

“Fe-fusion protein”, as used herein, encompass the polypeptides comprising an immunoglobin Fc domain that is directly linked to another peptide, or linked to another peptide using a linker.

“Framework region” or “FR”, as used herein, includes the amino acid residues that are part of the variable region, but are not part of the CDRs (e.g., using the Kabat/Chothia definition of CDRs). Therefore, a variable region framework is between about 100-120 amino acids in length but includes only those amino acids outside of the CDRs.

For the specific example of a HCVR and for the CDRs as defined by Kabat/Chothia:
FR1 may correspond to the domain of the variable region encompassing amino acids 1-25 according to Chothia/AbM's definition, or 5 residues later according to Kabat's definition;
FR2 may correspond to the domain of the variable region encompassing amino acids 36-49;
FR3 may correspond to the domain of the variable region encompassing amino acids 67-98; and
FR4 may correspond to the domain of the variable region from amino acids 104-110 to the end of the variable region.

The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Heavy chain region”, as used herein, includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or binding fragment thereof according to the present invention may comprise the Fc region of an immunoglobulin heavy chain (e.g., a hinge portion, a C_{H}2 domain, and a C_{H}3 domain). In another embodiment, the antibody or binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the antibody or binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

“Hinge region”, as used herein, includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

“Human serum albumin fusion protein”, as used herein, refers to a recombinant protein composed of a peptide fused to the at least one domain of the human serum albumin using or not a linker.

“Humanized antibody”, as used herein, refers to a chimeric antibody or binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody” also includes antibodies and binding fragment thereof in which CDR sequences derived from the germline of another mammalian species, such as a mouse, have been grafted onto human framework sequences. In other words, the term “humanized antibody” refers to an antibody or binding fragment thereof in which the CDRs of a recipient human antibody are replaced by CDRs from a donor non-human antibody, e.g., a mouse antibody. Humanized antibodies or binding fragments thereof may also comprise residues of donor origin in the framework sequences. The humanized antibody or binding fragment thereof can also comprise at least a portion of a human immunoglobulin constant region. Humanized antibodies and binding fragments thereof may also comprise residues which are found neither in the recipient antibody, nor in the imported CDR or FR sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Natl Acad Sci USA. 91(3):969-73).

Methods for humanizing the antibody or binding fragment thereof according to the present invention are well-known in the art. The choice of human variable domains, both light and heavy, to be used in making the humanized antibody or binding fragment thereof is very important to reduce antigenicity. According to the so-called “best-fit” method, the sequence of the variable domain of an antibody or binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or binding fragment thereof according to the present invention uses a particular FR from the consensus sequence of all human antibodies of a particular subgroup of light or heavy chains. The same framework can be used for several different humanized antibodies (Carter et al., 1992. Proc Natl Acad Sci USA. 89(10):4285-9; Presta et al., 1993. J Immunol. 151(5):2623-32). It is further important that antibodies be humanized with retention of high affinity for CD38 and other favorable biological properties. To achieve this goal, according to a preferred method, humanized antibodies and binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for CD38, is achieved. In general, the CDR residues are directly and most substantially involved in influencing antigen binding.

Another method for humanizing the antibody or binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized Examples of such transgenic or transchromosomic animal include, without limitation:
the XenoMouse (Abgenix, Fremont, Calif.), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in Patent application WO2002043478;
the TC mice, described in Tomizuka et al., 2000. Proc Natl Acad Sci USA. 97(2): 722-7; and
the OmniRat™ (OMT, Inc.), described in Patent application WO2008151081; Geurts et al., 2009. Science. 325(5939):433; Menoret et al., 2010. Eur J Immunol. 40(10):2932-41; Osborn et al., 2013. J Immunol. 190(4):1481-90.

Humanized antibodies and binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or binding fragments thereof as disclosed in the present application.

In some embodiments, the antibody or binding fragment thereof according to the present invention comprising HCVR and LCVR (or CDRs thereof) may comprise a first constant domain (C_{H}1 and/or C_{L}), the amino acid sequence of which is fully or substantially human. In some embodiment, especially when the antibody or binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required. The presence of a “fully human hinge region” in the antibody or binding fragment thereof according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or binding fragment thereof, it may be desirable to modify the antibody or binding fragment thereof according to the present invention with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

“Hypervariable loop”, as used herein, is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, Md.: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

“Identity” or “identical”, when used in a relationship between the sequences of two or more amino acid sequences, or of two or more nucleic acid sequences, refer to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”).

Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82.

Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Medicament”, as used herein, is meant to encompass a composition suitable for administration to a subject or patient, such as a mammal, especially a human. In general, a “medicament” is sterile and is usually free of contaminants that are capable of eliciting an undesirable response within the subject (e.g., the compound(s) in the medicament is pharmaceutical grade). Medicaments can be designed for administration to subjects in need thereof via a number of different routes of administration including oral, buccal, rectal, parenteral, intraperitoneal, intradermal, subcutaneous, intranasal, intrathecal, perispinal and the like. The preferred form depends on the intended mode of administration and therapeutic application. Typical preferred form are oral, injectable or infusible solutions.

“Modified antibody”, as used herein, corresponds to a molecule comprising an antibody or an antigen-binding fragment thereof, wherein said antibody or fragment thereof is associated with a functionally different molecule. A modified antibody of the invention may be either a fusion chimeric protein or a conjugate resulting from any suitable form of attachment including covalent attachment, grafting, chemical bonding with a chemical or biological group or with a molecule, such as a PEG polymer or another protective group or molecule suitable for protection against proteases cleavage in vivo, for improvement of stability and/or half-life of the antibody or functional fragment. With similar techniques, especially by chemical coupling or grafting, a modified antibody can be prepared with a biologically active molecule, said active molecule being for example chosen among toxins, in particular Pseudomonas exotoxin A, the A-chain of plant toxin ricin or saporin toxin, especially a therapeutic active ingredient, a vector (including especially a protein vector) suitable for targeting the antibody or functional fragment to specific cells or tissues of the human body, or it may be associated with a label or with a linker, especially when fragments of the antibody are used. PEGylation of the antibody or functional fragments thereof is a particular interesting embodiment as it improves the delivery conditions of the active substance to the host, especially for a therapeutic application. PEGylation can be site specific to prevent interference with the recognition sites of the antibodies or functional fragments, and can be performed with high molecular weight PEG. PEGylation can be achieved through free cysteine residues present in the sequence of the antibody or functional fragment or through added free Cysteine residues in the amino sequence of the antibody or functional fragment.

“Monoclonal antibody”, as used herein, is intended to refer to a preparation of antibody molecules, which share a common heavy chain and common light chain amino acid sequence, in contrast with “polyclonal antibody” preparations which contain a mixture of antibodies of different amino acid sequence. Certain differences in protein sequences, related to post-translational modifications (such as for example the cleavage of the C-terminal lysine of the heavy chain, deamidation of asparagine residues and/or isomerization of aspartate residues), may nevertheless exist between individual antibodies present in a monoclonal antibody composition. The modifier “monoclonal” is not to be construed as requiring production of the antibody by any particular method. Monoclonal antibodies can be generated by several known technologies like phage, bacteria, yeast or ribosomal display, as well as by classical methods exemplified by hybridoma-derived antibodies, first described by Kohler et al. (1975. Nature. 256(5517):495-7). Thus, the term “monoclonal” is used to refer to all antibodies derived from one nucleic acid clone.

“Oligonucleotide”, as used herein, relates to a short molecule of nucleotides including DNA, RNA but also modified nucleotides (such as nucleotides comprising at least one chemical modification). In particular, said oligonucleotide consists of less than 200 nucleotides, more particularly of less than 50 nucleotides.

“Oligonucleotide aptamer”, as used herein, refers to short oligonucleotides that can selectively bind to small molecular ligands or protein targets with high affinity and specificity, when folded into their unique three-dimensional structures.

“Peptide”, as used herein, is intended to refer to a molecule that is at least composed of amino acids. A peptide can also possess other molecular groups like polysaccharide chains or other post-translational modifications. “Recombinant peptide” specifically refers to a peptide which is produced, expressed, generated or isolated by recombinant means, such as peptides which are expressed using a recombinant expression vector transfected into a host cell. Recombinant peptides include, for example, chimeric peptides. “Chimeric peptide” is intended to refer to a peptide that is created through the joining of two or more genes that originally coded for separate proteins or protein fragments; or through the fusion of two or more proteins or protein fragments. Chimeric peptides include, for example, a peptide fused to the Fc region of an IgG, a peptide fused to human serum albumin (HSA) or a particular domain (such as, e.g., domain III) of HSA, or to transferrin (as in Strohl, 2015. BioDrugs. 29(4): 215-239.

“Peptide aptamers”, as used herein, refers to small combinatorial proteins that are selected to bind to specific sites on their target antigens.

“Recombinant antibody”, as used herein, refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e g, a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

“Small organic molecule”, as used herein, refers to a low molecular weight organic compound (up to 5000 Da, more in particular up to 2000 Da, and most in particular up to about 1000 Da).

“Subject”, as used herein, refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

“Therapeutically effective amount”, as used herein, means the level, amount or concentration of agent (e.g., a compound that competes with CD31 for CD38 binding) that is aimed at, without causing significant negative or adverse side effects to the subject, (1) delaying or preventing the onset of the targeted disease(s); (2) slowing down or stopping the progression, aggravation, or deterioration of one or more symptoms of the targeted disease(s); (3) bringing about ameliorations of the symptoms of the targeted disease(s); (4) reducing the severity or incidence of the targeted disease(s); or (5) curing the targeted disease(s). A therapeutically effective amount may be administered prior to the onset of the targeted disease(s), for a prophylactic or preventive action. Alternatively or additionally, the therapeutically effective amount may be administered after initiation of the targeted disease(s), for a therapeutic action.

“Treating” or “treatment” or “alleviation”, as used herein, refer to both therapeutic and prophylactic (or preventative) measures; wherein the object is to slow down (or lessen) the targeted disease(s). Those in need of treatment include those already with the targeted disease(s) as well those suspected to have the targeted disease(s).

“Variable region” or “variable domain”, as used herein, refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site.

The first, second and third hypervariable loops of the Vλ light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the V_{L} domain (Morea et al., 2000. Methods. 20(3):267-79).

The first, second and third hypervariable loops of the V_{κ} light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., 2000. Methods. 20(3):267-79).

The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the V_{H} domain (Morea et al., 2000. Methods. 20(3):267-79).

Unless otherwise indicated, the terms L1, L2 and L3 respectively refer to the first, second and third hypervariable loops of a V_{L} domain, and encompass hypervariable loops obtained from both V_{κ} and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

### DETAILED DESCRIPTION

The present invention relates to a compound which specifically competes with CD31 for CD38 binding.

In one embodiment, the compound according to the present invention competes with the extracellular domain of CD31. In one embodiment, the compound according to the present invention competes with the Ig-like domains 1-3 of the extracellular domain of CD31.

The amino acid sequence of human CD31 set forth in SEQ ID NO: 1 comprises the following features: a signal peptide from amino acid 1 to 27; the Ig-like domain 1 from amino acid 34 to 121; the Ig-like domain 2 from amino acid 145 to 233; the Ig-like domain 3 from amino acid 236 to 315; the Ig-like domain 4 from amino acid 328 to 401; the Ig-like domain 5 from amino acid 424 to 493; the Ig-like domain 6 from amino acid 499 to 591; a juxta-membrane domain from amino acid 592 to 601; a transmembrane domain from amino acid 602 to 620 and a cytoplasmic domain from amino acid 621 to 738.

Accordingly, in one embodiment, the compound according to the present invention competes with the extracellular domain of human CD31, preferably with amino acids 1 to 601 or 28 to 601 of SEQ ID NO: 1. In one embodiment, the compound according to the present invention competes with the Ig-like domains 1-3 of human CD31, preferably with amino acids 1 to 315 or 28 to 315 or 1 to 327 or 28 to 327 of SEQ ID NO: 1.

In one embodiment, the compound according to the present invention is selected from the group comprising or consisting of a peptide (including a recombinant peptide or a chimeric peptide), an antibody, an antigen-binding fragment thereof, an antigen-binding antibody mimetic, an oligonucleotide and a small organic molecule.

In one embodiment, the compound according to the present invention specifically binds to CD38, preferably to human CD38.

The specific binding between the compound according to the invention and CD38 implies that said compound exhibits appreciable affinity for CD38.

The affinity of the compound according to the invention for CD38 can be determined by various methods well known from the one skilled in the art. These methods include, but are not limited to, biosensor analysis (including, e.g., Biacore analysis), Blitz analysis and Scatchard plot.

Alternatively or additionally, whether the compound according to the present invention binds to CD38 can be tested readily by, inter alia, comparing the reaction of said compound with CD38 or a fragment thereof (in particular, a fragment comprising or consisting of an epitope of CD38) with the reaction of said compound with proteins or antigens other than CD38 or a fragment thereof.

In one embodiment where the compound according to the present invention is an antibody, an antigen-binding fragment thereof or an antigen-binding antibody mimetic, “appreciable affinity” may be defined by a K_{D}-affinity constant of about 10⁻⁷ M, preferably of about 10⁻⁸ M or stronger. In particular, the binding between the antibody, antigen-binding fragment thereof or antigen-binding antibody mimetic according to the invention and CD38 is considered to be specific when the K_{D}-affinity constant ranges from about 10⁻⁷ M to about 10⁻¹² M, preferably from about 10⁻⁸ to about 10⁻¹², more preferably from about 10⁻⁹ M to about 10⁻¹¹ M, in particular the K_{D}-affinity constant is of about 10⁻¹⁰ M.

In one embodiment where the compound according to the present invention is an antibody, an antigen-binding fragment thereof or an antigen-binding antibody mimetic, said compound has a K_{D}-affinity constant inferior or equal to about 10⁻⁷ M, preferably inferior or equal to about 10⁻⁸, more preferably inferior or equal to about 10⁻⁹ M, even more preferably inferior or equal to about 10⁻¹⁰ M for CD38; preferably as may be determined by biosensor analysis, particularly by Biacore Analysis.

In one embodiment where the compound according to the present invention is a small organic molecule, “appreciable affinity” may be defined by a K_{D}-affinity constant of about 10⁻⁶ M. In particular, the binding between the small organic molecule according to the invention and CD38 is considered to be specific when the K_{D}-affinity constant ranges from about 10⁻⁶ M to about 10⁻¹⁰ M, preferably from about 10⁻⁷ M to about 10⁻⁹ M, in particular the K_{D}-affinity constant is of about 10⁻⁸M; preferably as may be determined by biosensor analysis, particularly by Biacore Analysis.

In one embodiment where the compound according to the present invention is an oligonucleotide, “appreciable affinity” may be defined by a K_{D}-affinity constant of about 200 nM or stronger. In particular, the binding between the oligonucleotide according to the invention and CD38 is considered to be specific when the K_{D}-affinity constant ranges from about 10 nM to about 200 nM, preferably from about 50 nM to about 150 nM, in particular the K_{D}-affinity constant is of about 100 nM; preferably as may be determined by biosensor analysis, particularly by Biacore Analysis.

In one embodiment, the compound according to the present invention specifically binds to at least one epitope within CD38.

In one embodiment, the compound according to the present invention specifically binds to at least one epitope within human CD38.

In one embodiment, said epitope is linear. In one embodiment, said epitope is conformational.

In one embodiment, said epitope comprises 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 contiguous or non-contiguous amino acid residues.

In one embodiment, said epitope extends at least throughout amino acid residue 1 to amino acid residue 300 of human CD38 with SEQ ID NO: 2, or throughout the corresponding amino acid residues in homologous CD38 from other species.

In one embodiment, said epitope extends at least throughout amino acid residue 1 to amino acid residue 296 of human CD38 with SEQ ID NO: 2, or throughout the corresponding amino acid residues in homologous CD38 from other species.

In one embodiment, said epitope extends at least throughout amino acid residue 1 to amino acid residue 294 of human CD38 with SEQ ID NO: 2, or throughout the corresponding amino acid residues in homologous CD38 from other species.

In one embodiment, said epitope extends at least throughout amino acid residue 1 to amino acid residue 285 of human CD38 with SEQ ID NO: 2, or throughout the corresponding amino acid residues in homologous CD38 from other species.

In one embodiment, said epitope extends at least throughout amino acid residue 70 to amino acid residue 300 of human CD38 with SEQ ID NO: 2, or throughout the corresponding amino acid residues in homologous CD38 from other species.

In one embodiment, said epitope extends at least throughout amino acid residue 70 to amino acid residue 296 of human CD38 with SEQ ID NO: 2, or throughout the corresponding amino acid residues in homologous CD38 from other species.

In one embodiment, said epitope extends at least throughout amino acid residue 70 to amino acid residue 294 of human CD38 with SEQ ID NO: 2, or throughout the corresponding amino acid residues in homologous CD38 from other species.

In one embodiment, said epitope extends at least throughout amino acid residue 70 to amino acid residue 285 of human CD38 with SEQ ID NO: 2, or throughout the corresponding amino acid residues in homologous CD38 from other species.

In one embodiment, said epitope extends at least throughout amino acid residue 189 to amino acid residue 300 of human CD38 with SEQ ID NO: 2, or throughout the corresponding amino acid residues in homologous CD38 from other species.

In one embodiment, said epitope extends at least throughout amino acid residue 189 to amino acid residue 296 of human CD38 with SEQ ID NO: 2, or throughout the corresponding amino acid residues in homologous CD38 from other species.

In one embodiment, said epitope extends at least throughout amino acid residue 189 to amino acid residue 294 of human CD38 with SEQ ID NO: 2, or throughout the corresponding amino acid residues in homologous CD38 from other species.

In one embodiment, said epitope extends at least throughout amino acid residue 189 to amino acid residue 285 of human CD38 with SEQ ID NO: 2, or throughout the corresponding amino acid residues in homologous CD38 from other species.

In one embodiment, said epitope extends at least throughout amino acid residue 219 to amino acid residue 300 of human CD38 with SEQ ID NO: 2, or throughout the corresponding amino acid residues in homologous CD38 from other species.

In one embodiment, said epitope extends at least throughout amino acid residue 219 to amino acid residue 296 of human CD38 with SEQ ID NO: 2, or throughout the corresponding amino acid residues in homologous CD38 from other species.

In one embodiment, said epitope extends at least throughout amino acid residue 219 to amino acid residue 294 of human CD38 with SEQ ID NO: 2, or throughout the corresponding amino acid residues in homologous CD38 from other species.

In one embodiment, said epitope extends at least throughout amino acid residue 219 to amino acid residue 285 of human CD38 with SEQ ID NO: 2, or throughout the corresponding amino acid residues in homologous CD38 from other species.

In one embodiment, said epitope extends at least throughout amino acid residue 220 to amino acid residue 300 of human CD38 with SEQ ID NO: 2, or throughout the corresponding amino acid residues in homologous CD38 from other species.

In one embodiment, said epitope extends at least throughout amino acid residue 220 to amino acid residue 296 of human CD38 with SEQ ID NO: 2, or throughout the corresponding amino acid residues in homologous CD38 from other species.

In one embodiment, said epitope extends at least throughout amino acid residue 220 to amino acid residue 294 of human CD38 with SEQ ID NO: 2, or throughout the corresponding amino acid residues in homologous CD38 from other species.

In one embodiment, said epitope extends at least throughout amino acid residue 220 to amino acid residue 285 of human CD38 with SEQ ID NO: 2, or throughout the corresponding amino acid residues in homologous CD38 from other species.

In one embodiment, said epitope extends at least throughout amino acid residue 233 to amino acid residue 300 of human CD38 with SEQ ID NO: 2, or throughout the corresponding amino acid residues in homologous CD38 from other species.

In one embodiment, said epitope extends at least throughout amino acid residue 233 to amino acid residue 296 of human CD38 with SEQ ID NO: 2, or throughout the corresponding amino acid residues in homologous CD38 from other species.

In one embodiment, said epitope extends at least throughout amino acid residue 233 to amino acid residue 294 of human CD38 with SEQ ID NO: 2, or throughout the corresponding amino acid residues in homologous CD38 from other species.

In one embodiment, said epitope extends at least throughout amino acid residue 233 to amino acid residue 285 of human CD38 with SEQ ID NO: 2, or throughout the corresponding amino acid residues in homologous CD38 from other species.

In one embodiment, said epitope extends at least throughout amino acid residue 220 to amino acid residue 241 and amino acid residue 273 to amino acid residue 285 of human CD38 with SEQ ID NO: 2, or throughout the corresponding amino acid residues in homologous CD38 from other species.

In one embodiment, said epitope comprises at least cysteine 254 of human CD38 with SEQ ID NO: 2 or the corresponding cysteine in homologous CD38 from other species.

In one embodiment, said epitope comprises at least cysteine 275 of human CD38 with SEQ ID NO: 2 or the corresponding cysteine in homologous CD38 from other species.

In one embodiment, said epitope comprises at least cysteine 254 and cysteine 275 of human CD38 with SEQ ID NO: 2 or the corresponding cysteine in homologous CD38 from other species.

In one embodiment, said epitope comprises the 5^{th} (penultimate) C-terminal disulfide loop involving cysteine 254 and cysteine 275 of human CD38 with SEQ ID NO: 2 or the corresponding cysteine in homologous CD38 from other species. In particular, the 5^{th} (penultimate) C-terminal disulfide loop of human CD38 comprises amino acid residues 220 to amino acid residue 285 of human CD38 with SEQ ID NO: 2, or the corresponding cysteine in homologous CD38 from other species.

In one embodiment, said epitope does not comprise cysteine 287 of human CD38 with SEQ ID NO: 2 or the corresponding cysteine in homologous CD38 from other species.

In one embodiment, said epitope does not comprise cysteine 290 of human CD38 with SEQ ID NO: 2 or the corresponding cysteine in homologous CD38 from other species.

In one embodiment, said epitope does not comprise cysteine 287 and cysteine 290 of human CD38 with SEQ ID NO: 2 or the corresponding cysteine in homologous CD38 from other species.

In one embodiment, said epitope does not comprise the 6^{th} C-terminal disulfide loop involving cysteine 287 and cysteine 296 of human CD38 with SEQ ID NO: 2 or the corresponding cysteine in homologous CD38 from other species. In particular, the 6^{th} C-terminal disulfide loop of human CD38 comprises amino acid residues 285 to amino acid residue 300 of human CD38 with SEQ ID NO: 2, or the corresponding cysteine in homologous CD38 from other species.

In one embodiment, the compound according to the present invention directly protects dopaminergic neurons from mitochondrial inhibition induced by the neurotoxin MPP⁺.

In one embodiment, the compound according to the present invention has at least one, preferably at least two, more preferably the three following properties:
it protects dopaminergic neurons against energy deficit due to mitochondrial complex I inhibition, in particular against the mitochondrial neurotoxin MPP⁺;
it increases the release of the anti-inflammatory cytokine interleukin-10 (IL-10) in vitro using human peripheral blood mononuclear cells (PBMC); and/or
it increases IL-10 levels in vivo when injected to mice.

In one embodiment, the compound according to the present invention competes with CD31, preferably with human CD31, and induces tyrosine phosphorylation. In particular, the compound according to the present invention induces tyrosine phosphorylation of discrete cytoplasmic substrates that is inhibited in the presence of genistein.

Examples of discrete cytoplasmic substrates phosphorylated include, but are not limited to, the c-cbl proto-oncogene, the protein kinase syk, the p85 subunit of phosphatidylinositol-3 kinase, phospholipase C-γ (PLC-γ), the Raf-1/MAP kinase, the CD3-ζ/ZAP-70 signaling pathway.

In one embodiment, the compound according to the present invention competes with CD31, preferably with human CD31, and triggers CD38 internalization.

In one embodiment, the compound according to the present invention competes with CD31, preferably with human CD31, and induces lysosomal exocytosis. In particular, the compound according to the present invention induces lysosomal exocytosis that is inhibited in the presence of vacuolin-1.

In one embodiment, the compound according to the present invention does not mediate, trigger or enhance antibody-dependent cellular cytotoxicity (ADCC), antibody-dependent cellular phagocytosis (ADCP) and/or complement-dependent cytotoxicity (CDC).

In one embodiment, the compound according to the present invention is a CD31 peptide, preferably a human CD31 peptide.

In one embodiment, the compound according to the present invention is a peptide comprising or consisting of the extracellular domain of human CD31. In one embodiment, the compound according to the present invention is a fragment of the extracellular domain of human CD31. In one embodiment, the compound according to the present invention is a peptide comprising or consisting of the Ig-like domains 1-3 of human CD31.

In one embodiment, the compound according to the present invention is a peptide comprising or consisting of amino acids 1 to 601 of SEQ ID NO: 1 or a variant thereof. In one embodiment, the compound according to the present invention is a peptide comprising or consisting of amino acids 28 to 601 of SEQ ID NO: 1 or a variant thereof. In one embodiment, the compound according to the present invention is a peptide comprising or consisting of amino acids 1 to 591 of SEQ ID NO: 1 or a variant thereof. In one embodiment, the compound according to the present invention is a peptide comprising or consisting of amino acids 28 to 591 of SEQ ID NO: 1 or a variant thereof. In one embodiment, the compound according to the present invention is a peptide comprising or consisting of amino acids 1 to 493 of SEQ ID NO: 1 or a variant thereof. In one embodiment, the compound according to the present invention is a peptide comprising or consisting of amino acids 28 to 493 of SEQ ID NO: 1 or a variant thereof. In one embodiment, the compound according to the present invention is a peptide comprising or consisting of amino acids 1 to 401 of SEQ ID NO: 1 or a variant thereof. In one embodiment, the compound according to the present invention is a peptide comprising or consisting of amino acids 28 to 401 of SEQ ID NO: 1 or a variant thereof. In one embodiment, the compound according to the present invention is a peptide comprising or consisting of amino acids 1 to 315 of SEQ ID NO: 1 or a variant thereof. In one embodiment, the compound according to the present invention is a peptide comprising or consisting of amino acids 28 to 315 of SEQ ID NO: 1 or a variant thereof. In one embodiment, the compound according to the present invention is a peptide comprising or consisting of amino acids 1 to 233 of SEQ ID NO: 1 or a variant thereof. In one embodiment, the compound according to the present invention is a peptide comprising or consisting of amino acids 28 to 233 of SEQ ID NO: 1 or a variant thereof. In one embodiment, the compound according to the present invention is a peptide comprising or consisting of amino acids 1 to 121 of SEQ ID NO: 1 or a variant thereof. In one embodiment, the compound according to the present invention is a peptide comprising or consisting of amino acids 28 to 121 of SEQ ID NO: 1 or a variant thereof.

In one embodiment, a variant of a peptide as described hereinabove shares at least 70%, preferably at least 75%, 80%, 85%, 90%, 95%, 96%, 97%, 98%, 99% or more local identity with said peptide.

In one embodiment, the compound according to the present invention is a chimeric CD31 peptide, preferably a chimeric human CD31 peptide.

In one embodiment, the chimeric CD31 peptide comprises or consists of a CD31 peptide as defined hereinabove, fused to a Fc domain of an immunoglobulin to form a Fc-fusion protein.

In one embodiment, the chimeric CD31 peptide comprises or consists of a CD31 peptide as defined hereinabove, fused to human serum albumin to form a human serum albumin fusion protein.

In one embodiment, the chimeric CD31 peptide comprises a CD31 peptide as defined hereinabove, fused to one or several domains of human serum albumin to form a human serum albumin domain fusion protein. In particular, the chimeric CD31 peptide may comprise or consist of a CD31 peptide as defined hereinabove, fused to the domain III of human serum albumin.

As used herein, the term “domain III of human serum albumin” comprises or consists of amino acid residues 404 to 601 of the sequence set forth in SEQ ID NO: 43, corresponding to human serum albumin encoded by the ALB gene (Uniprot accession number and version: P02768-1, last modified on Apr. 1, 1990; Checksum: F88FF61DD242E818).

In one embodiment, the chimeric CD31 peptide comprises or consists of a CD31 peptide as defined hereinabove, fused to transferrin to form a transferrin-fusion protein.

In one embodiment, the compound according to the present invention is an anti-CD38 antibody or an antigen-binding fragment thereof.

In one embodiment, the compound according to the present invention is an anti-CD38 polyclonal antibody or an antigen-binding fragment thereof.

In one embodiment, the compound according to the present invention is an anti-CD38 monoclonal antibody or an antigen-binding fragment thereof.

In one embodiment, the anti-CD38 antibody or antigen-binding fragment thereof according to the present invention is selected from the group comprising or consisting of mouse anti-human CD38 monoclonal antibody clone HB7; rat anti-human CD38 monoclonal antibody clone 90; mouse anti-human CD38 monoclonal antibody clone AT1; mouse anti-human CD38 monoclonal antibody clone T16; mouse anti-human CD38 monoclonal antibody clone IB4; and mutated, recombinant (including chimeric and humanized), bispecific and modified antibodies thereof which are able to specifically compete with CD31.

In one embodiment, the anti-CD38 antibody or antigen-binding fragment thereof according to the present invention is selected from the group comprising or consisting of mouse anti-human CD38 monoclonal antibody clone HB7; rat anti-human CD38 monoclonal antibody clone 90; and mutated, recombinant (including chimeric and humanized), bispecific and modified antibodies thereof which are able to specifically compete with CD31.

In one embodiment, the anti-CD38 antibody or antigen-binding fragment thereof according to the present invention is selected from the group comprising or consisting of mouse anti-human CD38 monoclonal antibody clone HB7; and mutated, recombinant (including chimeric and humanized), bispecific and modified antibodies thereof which are able to specifically compete with CD31.

In the following, and unless explicitly mentioned otherwise, CDR numbering and definitions are according to the Chothia definition.

In one embodiment, the anti-CD38 antibody or antigen-binding fragment thereof according to the present invention comprises a heavy chain variable region (abbreviated herein as HCVR or V_{H}) which comprises at least one, preferably at least two, more preferably the three following complementary-determining regions (CDRs):
V_{H}-CDR1: GFTFSNX₁ (SEQ ID NO: 4) or a variant thereof;
V_{H}-CDR2: X₂GSSRX₃ (SEQ ID NO: 5) or a variant thereof; and
V_{H}-CDR3: X₄X₅X₆X₇X₈YX₉X₁₀X₁₁X₁₂GMDV (SEQ ID NO: 6) or a variant thereof, with:
X₁ being selected from Tyr (Y), Asn (N) and Ser (S);
X₂ being selected from Ser (S) and Tyr (Y);
X₃ being selected from Tyr (Y), Asp (D), Asn (N) and Ser (S);
X₄ being selected from Ser (S) and an empty position;
X₅ being selected from Ser (S) and an empty position;
X₆ being selected from Ser (S) and Tyr (Y);
X₇ being selected from Ser (S) and Asp (D);
X₈ being selected from Tyr (Y), Ser (S), Asp (D) and Gly (G);
X₉ being selected from Tyr (Y) and Gly (G);
X₁₀ being selected from Ser (S), Tyr (Y) and Phe (F);
X₁₁ being selected from Gly (G) and Asp (D); and
X₁₂ being selected from Asn (N), Tyr (Y) and Ser (S).

In one embodiment, the anti-CD38 antibody or antigen-binding fragment thereof according to the present invention comprises a HCVR which comprises the three following CDRs:
V_{H}-CDR1: GFTFSNX₁ (SEQ ID NO: 4) or a variant thereof;
V_{H}-CDR2: X₂GSSRX₃ (SEQ ID NO: 5) or a variant thereof; and
V_{H}-CDR3: X₄X₅X₆X₇X₈YX₉X₁₀X₁₁X₁₂GMDV (SEQ ID NO: 6) or a variant thereof, with:
X₁ being selected from Tyr (Y), Asn (N) and Ser (S);
X₂ being selected from Ser (S) and Tyr (Y);
X₃ being selected from Tyr (Y), Asp (D), Asn (N) and Ser (S);
X₄ being selected from Ser (S) and an empty position;
X₅ being selected from Ser (S) and an empty position;
X₆ being selected from Ser (S) and Tyr (Y);
X₇ being selected from Ser (S) and Asp (D);
X₈ being selected from Tyr (Y), Ser (S), Asp (D) and Gly (G);
X₉ being selected from Tyr (Y) and Gly (G);
X₁₀ being selected from Ser (S), Tyr (Y) and Phe (F);
X₁₁ being selected from Gly (G) and Asp (D); and
X₁₂ being selected from Asn (N), Tyr (Y) and Ser (S).

In one embodiment, the anti-CD38 antibody or antigen-binding fragment thereof according to the present invention is named “Clone B08” according to the Examples below, and comprises a HCVR which comprises at least one, preferably at least two, more preferably the three following CDRs:
V_{H}-CDR1: GFTFSNY (SEQ ID NO: 7) or a variant thereof;
V_{H}-CDR2: SGSSRS (SEQ ID NO: 10) or a variant thereof; and
V_{H}-CDR3: SSDDYYYDYGMDV (SEQ ID NO: 15) or a variant thereof.

In one embodiment, the Clone B08 comprises a HCVR which comprises the three following CDRs:
V_{H}-CDR1: GFTFSNY (SEQ ID NO: 7) or a variant thereof;
V_{H}-CDR2: SGSSRS (SEQ ID NO: 10) or a variant thereof; and
V_{H}-CDR3: SSDDYYYDYGMDV (SEQ ID NO: 15) or a variant thereof.

In one embodiment, the anti-CD38 antibody or antigen-binding fragment thereof according to the present invention is named “Clone C05” according to the Examples below, and comprises a HCVR which comprises at least one, preferably at least two, more preferably the three following CDRs:
V_{H}-CDR1: GFTFSNN (SEQ ID NO: 8) or a variant thereof;
V_{H}-CDR2: SGSSRY (SEQ ID NO: 11) or a variant thereof; and
V_{H}-CDR3: SSSSYYYSGNGMDV (SEQ ID NO: 16) or a variant thereof.

In one embodiment, the Clone C05 comprises a HCVR which comprises the three following CDRs:
V_{H}-CDR1: GFTFSNN (SEQ ID NO: 8) or a variant thereof;
V_{H}-CDR2: SGSSRY (SEQ ID NO: 11) or a variant thereof; and
V_{H}-CDR3: SSSSYYYSGNGMDV (SEQ ID NO: 16) or a variant thereof.

In one embodiment, the anti-CD38 antibody or antigen-binding fragment thereof according to the present invention is named “Clone C06” according to the Examples below, and comprises a HCVR which comprises at least one, preferably at least two, more preferably the three following CDRs:
V_{H}-CDR1: GFTFSNS (SEQ ID NO: 9) or a variant thereof;
V_{H}-CDR2: SGSSRN (SEQ ID NO: 12) or a variant thereof; and
V_{H}-CDR3: SSYSSYGSGNGMDV (SEQ ID NO: 17) or a variant thereof.

In one embodiment, the Clone C06 comprises a HCVR which comprises the three following CDRs:
V_{H}-CDR1: GFTFSNS (SEQ ID NO: 9) or a variant thereof;
V_{H}-CDR2: SGSSRN (SEQ ID NO: 12) or a variant thereof; and
V_{H}-CDR3: SSYSSYGSGNGMDV (SEQ ID NO: 17) or a variant thereof.

In one embodiment, the anti-CD38 antibody or antigen-binding fragment thereof according to the present invention is named “Clone D06” according to the Examples below, and comprises a HCVR which comprises at least one, preferably at least two, more preferably the three following CDRs:
V_{H}-CDR1: GFTFSNY (SEQ ID NO: 7) or a variant thereof;
V_{H}-CDR2: YGSSRD (SEQ ID NO: 13) or a variant thereof; and
V_{H}-CDR3: SSGYYFGYGMDV (SEQ ID NO: 19) or a variant thereof.

In one embodiment, the Clone D06 comprises a HCVR which comprises the three following CDRs:
V_{H}-CDR1: GFTFSNY (SEQ ID NO: 7) or a variant thereof;
V_{H}-CDR2: YGSSRD (SEQ ID NO: 13) or a variant thereof; and
V_{H}-CDR3: SSGYYFGYGMDV (SEQ ID NO: 19) or a variant thereof.

In one embodiment, the anti-CD38 antibody or antigen-binding fragment thereof according to the present invention is named “Clone D07” according to the Examples below, and comprises a HCVR which comprises at least one, preferably at least two, more preferably the three following CDRs:
V_{H}-CDR1: GFTFSNS (SEQ ID NO: 9) or a variant thereof;
V_{H}-CDR2: SGSSRD (SEQ ID NO: 14) or a variant thereof; and
V_{H}-CDR3: SSSSYYYSGSGMDV (SEQ ID NO: 20) or a variant thereof.

In one embodiment, the Clone D07 comprises a HCVR which comprises the three following CDRs:
V_{H}-CDR1: GFTFSNS (SEQ ID NO: 9) or a variant thereof;
V_{H}-CDR2: SGSSRD (SEQ ID NO: 14) or a variant thereof; and
V_{H}-CDR3: SSSSYYYSGSGMDV (SEQ ID NO: 20) or a variant thereof.

In one embodiment, the anti-CD38 antibody or antigen-binding fragment thereof according to the present invention is named “Clone D10” according to the Examples below, and comprises a HCVR which comprises at least one, preferably at least two, more preferably the three following CDRs:
V_{H}-CDR1: GFTFSNS (SEQ ID NO: 9) or a variant thereof;
V_{H}-CDR2: SGSSRD (SEQ ID NO: 14) or a variant thereof; and
V_{H}-CDR3: SSSSYYYSGNGMDV (SEQ ID NO: 16) or a variant thereof.

In one embodiment, the Clone D10 comprises a HCVR which comprises the three following CDRs:
V_{H}-CDR1: GFTFSNS (SEQ ID NO: 9) or a variant thereof;
V_{H}-CDR2: SGSSRD (SEQ ID NO: 14) or a variant thereof; and
V_{H}-CDR3: SSSSYYYSGNGMDV (SEQ ID NO: 16) or a variant thereof.

In one embodiment, the anti-CD38 antibody or antigen-binding fragment thereof according to the present invention comprises a light chain variable region (abbreviated herein as LCVR or V_{L}) which comprises at least one, preferably at least two, more preferably the three following complementary-determining regions (CDRs):
V_{L}-CDR1: AGTSSDVGGX₁₃X₁₄X₁₅VS (SEQ ID NO: 21) or a variant thereof;
V_{L}-CDR2: X₁₆DSX₁₇RPS (SEQ ID NO: 22) or a variant thereof; and
V_{L}-CDR3: STRVFGGGT (SEQ ID NO: 23) or a variant thereof,
with:
X₁₃ being selected from Ser (S) and Asn (N);
X₁₄ being selected from Ser (S) and Tyr (Y);
X₁₅ being selected from Tyr (Y), and Ser (S);
X₁₆ being selected from Tyr (Y), Ser (S) and Asp (D); and
X₁₇ being selected from Tyr (Y) and Asn (N).

In one embodiment, the anti-CD38 antibody or antigen-binding fragment thereof according to the present invention comprises a LCVR which comprises the three following CDRs:
V_{L}-CDR1: AGTSSDVGGX₁₃X₁₄X₁₅VS (SEQ ID NO: 21) or a variant thereof;
V_{L}-CDR2: X₁₆DSX₁₇RPS (SEQ ID NO: 22) or a variant thereof; and
V_{L}-CDR3: STRVFGGGT (SEQ ID NO: 23) or a variant thereof,
with:
X₁₃ being selected from Ser (S) and Asn (N);
X₁₄ being selected from Ser (S) and Tyr (Y);
X₁₅ being selected from Tyr (Y), and Ser (S);
X₁₆ being selected from Tyr (Y), Ser (S) and Asp (D); and
X₁₇ being selected from Tyr (Y) and Asn (N).

In one embodiment, the anti-CD38 antibody or antigen-binding fragment thereof according to the present invention is named “Clone B08” according to the Examples below, and comprises a LCVR which comprises at least one, preferably at least two, more preferably the three following CDRs:
V_{L}-CDR1: AGTSSDVGGSYSVS (SEQ ID NO: 24) or a variant thereof;
V_{L}-CDR2: DDSNRPS (SEQ ID NO: 29) or a variant thereof; and
V_{L}-CDR3: STRVFGGGT (SEQ ID NO: 23) or a variant thereof.

In one embodiment, the Clone B08 comprises a LCVR which comprises the three following CDRs:
V_{L}-CDR1: AGTSSDVGGSYSVS (SEQ ID NO: 24) or a variant thereof;
V_{L}-CDR2: DDSNRPS (SEQ ID NO: 29) or a variant thereof; and
V_{L}-CDR3: STRVFGGGT (SEQ ID NO: 23) or a variant thereof.

In one embodiment, the anti-CD38 antibody or antigen-binding fragment thereof according to the present invention is named “Clone C05” according to the Examples below, and comprises a LCVR which comprises at least one, preferably at least two, more preferably the three following CDRs:
V_{L}-CDR1: AGTSSDVGGSSYVS (SEQ ID NO: 25) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the Clone C05 comprises a LCVR which comprises the three following CDRs:
V_{L}-CDR1: AGTSSDVGGSSYVS (SEQ ID NO: 25) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the anti-CD38 antibody or antigen-binding fragment thereof according to the present invention is named “Clone C06” according to the Examples below, and comprises a LCVR which comprises at least one, preferably at least two, more preferably the three following CDRs:
V_{L}-CDR1: AGTSSDVGGSSYVS (SEQ ID NO: 25) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the Clone C06 comprises a LCVR which comprises the three following CDRs:
V_{L}-CDR1: AGTSSDVGGSSYVS (SEQ ID NO: 25) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the anti-CD38 antibody or antigen-binding fragment thereof according to the present invention is named “Clone D06” according to the Examples below, and comprises a LCVR which comprises at least one, preferably at least two, more preferably the three following CDRs:
V_{L}-CDR1: AGTSSDVGGSYYVS (SEQ ID NO: 27) or a variant thereof;
V_{L}-CDR2: SDSYRPS (SEQ ID NO: 32) or a variant thereof; and
V_{L}-CDR3: STRVFGGGT (SEQ ID NO: 23) or a variant thereof.

In one embodiment, the Clone D06 comprises a LCVR which comprises the three following CDRs:
V_{L}-CDR1: AGTSSDVGGSYYVS (SEQ ID NO: 27) or a variant thereof;
V_{L}-CDR2: SDSYRPS (SEQ ID NO: 32) or a variant thereof; and
V_{L}-CDR3: STRVFGGGT (SEQ ID NO: 23) or a variant thereof.

In one embodiment, the anti-CD38 antibody or antigen-binding fragment thereof according to the present invention is named “Clone D07” according to the Examples below, and comprises a LCVR which comprises at least one, preferably at least two, more preferably the three following CDRs:
V_{L}-CDR1: AGTSSDVGGNSYVS (SEQ ID NO: 28) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the Clone D07 comprises a LCVR which comprises the three following CDRs:
V_{L}-CDR1: AGTSSDVGGNSYVS (SEQ ID NO: 28) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the anti-CD38 antibody or antigen-binding fragment thereof according to the present invention is named “Clone D10” according to the Examples below, and comprises a LCVR which comprises at least one, preferably at least two, more preferably the three following CDRs:
V_{L}-CDR1: AGTSSDVGGNSYVS (SEQ ID NO: 28) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the Clone D10 comprises a LCVR which comprises the three following CDRs:
V_{L}-CDR1: AGTSSDVGGNSYVS (SEQ ID NO: 28) or a variant thereof;
V_{L}-CDR2: YDSYRPS (SEQ ID NO: 30) or a variant thereof; and
V_{L}-CDR3: STRVFGGGT (SEQ ID NO: 23) or a variant thereof.

In one embodiment, the anti-CD38 antibody or antigen-binding fragment thereof according to the present invention comprises:
a HCVR which comprises at least one, preferably at least two, more preferably the three following CDRs:
   V_{H}-CDR1: GFTFSNX₁ (SEQ ID NO: 4) or a variant thereof,
   V_{H}-CDR2: X₂GSSRX₃ (SEQ ID NO: 5) or a variant thereof, and
   V_{H}-CDR3: X₄X₅X₆X₇X₈YX₉X₁₀X₁₁X₁₂GMDV (SEQ ID NO: 6) or a variant thereof; and
a LCVR which comprises at least one, preferably at least two, more preferably the three following CDRs:
   V_{L}-CDR1: AGTSSDVGGX₁₃X₁₄X₁₅VS (SEQ ID NO: 21) or a variant thereof;
   V_{L}-CDR2: X₁₆DSX₁₇RPS (SEQ ID NO: 22) or a variant thereof; and
   V_{L}-CDR3: STRVFGGGT (SEQ ID NO: 23) or a variant thereof;
   with:
   X₁ being selected from Tyr (Y), Asn (N) and Ser (S);
   X₂ being selected from Ser (S) and Tyr (Y);
   X₃ being selected from Tyr (Y), Asp (D), Asn (N) and Ser (S);
   X₄ being selected from Ser (S) and an empty position;
   X₅ being selected from Ser (S) and an empty position;
   X₆ being selected from Ser (S) and Tyr (Y);
   X₇ being selected from Ser (S) and Asp (D);
   X₈ being selected from Tyr (Y), Ser (S), Asp (D) and Gly (G);
   X₉ being selected from Tyr (Y) and Gly (G);
   X₁₀ being selected from Ser (S), Tyr (Y) and Phe (F);
   X₁₁ being selected from Gly (G) and Asp (D);
   X₁₂ being selected from Asn (N), Tyr (Y) and Ser (S);
   X₁₃ being selected from Ser (S) and Asn (N);
   X₁₄ being selected from Ser (S) and Tyr (Y);
   X₁₅ being selected from Tyr (Y), and Ser (S);
   X₁₆ being selected from Tyr (Y), Ser (S) and Asp (D); and
   X₁₇ being selected from Tyr (Y) and Asn (N).

In one embodiment, the anti-CD38 antibody or antigen-binding fragment thereof according to the present invention comprises:
a HCVR which comprises the three following CDRs:
   V_{H}-CDR1: GFTFSNX₁ (SEQ ID NO: 4) or a variant thereof,
   V_{H}-CDR2: X₂GSSRX₃ (SEQ ID NO: 5) or a variant thereof, and
   V_{H}-CDR3: X₄X₅X₆X₇X₈YX₉X₁₀X₁₁X₁₂GMDV (SEQ ID NO: 6) or a variant thereof; and
a LCVR which comprises the three following CDRs:
   V_{L}-CDR1: AGTSSDVGGX₁₃X₁₄X₁₅VS (SEQ ID NO: 21) or a variant thereof;
   V_{L}-CDR2: X₁₆DSX₁₇RPS (SEQ ID NO: 22) or a variant thereof; and
   V_{L}-CDR3: STRVFGGGT (SEQ ID NO: 23) or a variant thereof;
   with:
   X₁ being selected from Tyr (Y), Asn (N) and Ser (S);
   X₂ being selected from Ser (S) and Tyr (Y);
   X₃ being selected from Tyr (Y), Asp (D), Asn (N) and Ser (S);
   X₄ being selected from Ser (S) and an empty position;
   X₅ being selected from Ser (S) and an empty position;
   X₆ being selected from Ser (S) and Tyr (Y);
   X₇ being selected from Ser (S) and Asp (D);
   X₈ being selected from Tyr (Y), Ser (S), Asp (D) and Gly (G);
   X₉ being selected from Tyr (Y) and Gly (G);
   X₁₀ being selected from Ser (S), Tyr (Y) and Phe (F);
   X₁₁ being selected from Gly (G) and Asp (D);
   X₁₂ being selected from Asn (N), Tyr (Y) and Ser (S);
   X₁₃ being selected from Ser (S) and Asn (N);
   X₁₄ being selected from Ser (S) and Tyr (Y);
   X₁₅ being selected from Tyr (Y), and Ser (S);
   X₁₆ being selected from Tyr (Y), Ser (S) and Asp (D); and
   X₁₇ being selected from Tyr (Y) and Asn (N).

In one embodiment, the anti-CD38 antibody or antigen-binding fragment thereof according to the present invention is named “Clone B08” according to the Examples below, and comprises:
a HCVR which comprises at least one, preferably at least two, more preferably the three following CDRs:
   V_{H}-CDR1: GFTFSNY (SEQ ID NO: 7) or a variant thereof,
   V_{H}-CDR2: SGSSRS (SEQ ID NO: 10) or a variant thereof, and
   V_{H}-CDR3: SSDDYYYDYGMDV (SEQ ID NO: 15) or a variant thereof; and
a LCVR which comprises at least one, preferably at least two, more preferably the three following CDRs:
   V_{L}-CDR1: AGTSSDVGGSYSVS (SEQ ID NO: 24) or a variant thereof,
   V_{L}-CDR2: DDSNRPS (SEQ ID NO: 29) or a variant thereof, and
   V_{L}-CDR3: STRVFGGGT (SEQ ID NO: 23) or a variant thereof.

In one embodiment, the Clone B08 comprises:
a HCVR which comprises the three following CDRs:
   V_{H}-CDR1: GFTFSNY (SEQ ID NO: 7) or a variant thereof,
   V_{H}-CDR2: SGSSRS (SEQ ID NO: 10) or a variant thereof, and
   V_{H}-CDR3: SSDDYYYDYGMDV (SEQ ID NO: 15) or a variant thereof; and
a LCVR which comprises the three following CDRs:
   V_{L}-CDR1: AGTSSDVGGSYSVS (SEQ ID NO: 24) or a variant thereof,
   V_{L}-CDR2: DDSNRPS (SEQ ID NO: 29) or a variant thereof, and
   V_{L}-CDR3: STRVFGGGT (SEQ ID NO: 23) or a variant thereof.

In one embodiment, the anti-CD38 antibody or antigen-binding fragment thereof according to the present invention is named “Clone C05” according to the Examples below, and comprises:
a HCVR which comprises at least one, preferably at least two, more preferably the three following CDRs:
   V_{H}-CDR1: GFTFSNN (SEQ ID NO: 8) or a variant thereof,
   V_{H}-CDR2: SGSSRY (SEQ ID NO: 11) or a variant thereof, and
   V_{H}-CDR3: SSSSYYYSGNGMDV (SEQ ID NO: 16) or a variant thereof; and
a LCVR which comprises at least one, preferably at least two, more preferably the three following CDRs:
   V_{L}-CDR1: AGTSSDVGGSSYVS (SEQ ID NO: 25) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the Clone C05 comprises:
a HCVR which comprises the three following CDRs:
   V_{H}-CDR1: GFTFSNN (SEQ ID NO: 8) or a variant thereof,
   V_{H}-CDR2: SGSSRY (SEQ ID NO: 11) or a variant thereof, and
   V_{H}-CDR3: SSSSYYYSGNGMDV (SEQ ID NO: 16) or a variant thereof; and
a LCVR which comprises the three following CDRs:
   V_{L}-CDR1: AGTSSDVGGSSYVS (SEQ ID NO: 25) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the anti-CD38 antibody or antigen-binding fragment thereof according to the present invention is named “Clone C06” according to the Examples below, and comprises:
a HCVR which comprises at least one, preferably at least two, more preferably the three following CDRs:
   V_{H}-CDR1: GFTFSNS (SEQ ID NO: 9) or a variant thereof,
   V_{H}-CDR2: SGSSRN (SEQ ID NO: 12) or a variant thereof, and
   V_{H}-CDR3: SSYSSYGSGNGMDV (SEQ ID NO: 17) or a variant thereof; and
a LCVR which comprises at least one, preferably at least two, more preferably the three following CDRs:
   V_{L}-CDR1: AGTSSDVGGSSYVS (SEQ ID NO: 25) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the Clone C06 comprises:
a HCVR which comprises the three following CDRs:
   V_{H}-CDR1: GFTFSNS (SEQ ID NO: 9) or a variant thereof,
   V_{H}-CDR2: SGSSRN (SEQ ID NO: 12) or a variant thereof, and
   V_{H}-CDR3: SSYSSYGSGNGMDV (SEQ ID NO: 17) or a variant thereof; and
a LCVR which comprises the three following CDRs:
   V_{L}-CDR1: AGTSSDVGGSSYVS (SEQ ID NO: 25) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the anti-CD38 antibody or antigen-binding fragment thereof according to the present invention is named “Clone D06” according to the Examples below, and comprises:
a HCVR which comprises at least one, preferably at least two, more preferably the three following CDRs:
   V_{H}-CDR1: GFTFSNY (SEQ ID NO: 7) or a variant thereof,
   V_{H}-CDR2: YGSSRD (SEQ ID NO: 13) or a variant thereof, and
   V_{H}-CDR3: SSGYYFGYGMDV (SEQ ID NO: 19) or a variant thereof; and
a LCVR which comprises at least one, preferably at least two, more preferably the three following CDRs:
   V_{L}-CDR1: AGTSSDVGGSYYVS (SEQ ID NO: 27) or a variant thereof,
   V_{L}-CDR2: SDSYRPS (SEQ ID NO: 32) or a variant thereof, and
   V_{L}-CDR3: STRVFGGGT (SEQ ID NO: 23) or a variant thereof.

In one embodiment, the Clone D06 comprises:
a HCVR which comprises the three following CDRs:
   V_{H}-CDR1: GFTFSNY (SEQ ID NO: 7) or a variant thereof,
   V_{H}-CDR2: YGSSRD (SEQ ID NO: 13) or a variant thereof, and
   V_{H}-CDR3: SSGYYFGYGMDV (SEQ ID NO: 19) or a variant thereof; and
a LCVR which comprises the three following CDRs:
   V_{L}-CDR1: AGTSSDVGGSYYVS (SEQ ID NO: 27) or a variant thereof,
   V_{L}-CDR2: SDSYRPS (SEQ ID NO: 32) or a variant thereof, and
   V_{L}-CDR3: STRVFGGGT (SEQ ID NO: 23) or a variant thereof.

In one embodiment, the anti-CD38 antibody or antigen-binding fragment thereof according to the present invention is named “Clone D07” according to the Examples below, and comprises:
a HCVR which comprises at least one, preferably at least two, more preferably the three following CDRs:
   V_{H}-CDR1: GFTFSNS (SEQ ID NO: 9) or a variant thereof,
   V_{H}-CDR2: SGSSRD (SEQ ID NO: 14) or a variant thereof, and
   V_{H}-CDR3: SSSSYYYSGSGMDV (SEQ ID NO: 20) or a variant thereof; and
a LCVR which comprises at least one, preferably at least two, more preferably the three following CDRs:
   V_{L}-CDR1: AGTSSDVGGNSYVS (SEQ ID NO: 28) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the Clone D07 comprises:
a HCVR which comprises the three following CDRs:
   V_{H}-CDR1: GFTFSNS (SEQ ID NO: 9) or a variant thereof,
   V_{H}-CDR2: SGSSRD (SEQ ID NO: 14) or a variant thereof, and
   V_{H}-CDR3: SSSSYYYSGSGMDV (SEQ ID NO: 20) or a variant thereof; and
a LCVR which comprises the three following CDRs:
   V_{L}-CDR1: AGTSSDVGGNSYVS (SEQ ID NO: 28) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the anti-CD38 antibody or antigen-binding fragment thereof according to the present invention is named “Clone D10” according to the Examples below, and comprises:
a HCVR which comprises at least one, preferably at least two, more preferably the three following CDRs:
   V_{H}-CDR1: GFTFSNS (SEQ ID NO: 9) or a variant thereof,
   V_{H}-CDR2: SGSSRD (SEQ ID NO: 14) or a variant thereof, and
   V_{H}-CDR3: SSSSYYYSGNGMDV (SEQ ID NO: 16) or a variant thereof; and
a LCVR which comprises at least one, preferably at least two, more preferably the three following CDRs:
   V_{L}-CDR1: AGTSSDVGGNSYVS (SEQ ID NO: 28) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

In one embodiment, the Clone D10 comprises:
a HCVR which comprises the three following CDRs:
   V_{H}-CDR1: GFTFSNS (SEQ ID NO: 9) or a variant thereof,
   V_{H}-CDR2: SGSSRD (SEQ ID NO: 14) or a variant thereof, and
   V_{H}-CDR3: SSSSYYYSGNGMDV (SEQ ID NO: 16) or a variant thereof; and
a LCVR which comprises the three following CDRs:
   V_{L}-CDR1: AGTSSDVGGNSYVS (SEQ ID NO: 28) or a variant thereof,
   V_{L}-CDR2: YDSYRPS (SEQ ID NO: 30) or a variant thereof, and
   V_{L}-CDR3: STRVFGGGT (SEQ ID NO: 23) or a variant thereof.

A variant of a CDR refers to any of the CDRs as defined hereinabove characterized as having 1, 2 or 3 amino acids being substituted by a different amino acid.

A variant of a CDR refers to any of the CDRs as defined hereinabove characterized as having an amino acid sequence that shares at least 75%, 80%, 85%, 90%, 95%, 96%, 97%, 98%, 99% or more of identity with the particular CDR.

In one embodiment, the anti-CD38 antibody or antigen-binding fragment thereof according to the present invention is named “Clone B08” according to the Examples below, and comprises a HCVR with a sequence as set forth in SEQ ID NO: 33; or a variant thereof.

SEQ ID NO: 33
EVQLVESGGSLVKPGGSLRLSCAASGFTFSNYYMNWVRQAPGKGLEWISS
ISGSSRSIYYADFVKGRFTISRDNAKNSLYLQMNSLRAEDTAVYYCVRSS
DDYYYDYGMDVWGRGTLVTVSS

In one embodiment, the anti-CD38 antibody or antigen-binding fragment thereof according to the present invention is named “Clone C05” according to the Examples below, and comprises a HCVR with a sequence as set forth in SEQ ID NO: 34; or a variant thereof.

SEQ ID NO: 34
EVQLVESGGSLVKPGGSLRLSCAASGFTFSNNYMNWVRQAPGKGLEWISS
ISGSSRYISYADFVKGRFTISRDNAKNSLYLQMNSLRAEDTAVYYCVRSS
SSYYYSGNGMDVWGRGTLVTVSS

In one embodiment, the anti-CD38 antibody or antigen-binding fragment thereof according to the present invention is named “Clone C06” according to the Examples below, and comprises a HCVR with a sequence set forth in SEQ ID NO: 35; or a variant thereof.

SEQ ID NO: 35
EVQLVESGGSLVKPGGSLRLSCAASGFTFSNSAMNWVRQAPGKGLEWISS
ISGSSRNIYYADFVKGRFTISRDNAKNSLYLQMNSLRAEDTAVYYCVRSS
YSSYGSGNGMDVWGRGTLVTVSS

In one embodiment, the anti-CD38 antibody or antigen-binding fragment thereof according to the present invention is named “Clone D06” according to the Examples below, and comprises a HCVR with a sequence set forth in SEQ ID NO: 36; or a variant thereof.

SEQ ID NO: 36
EVQLVESGGSLVKPGGSLRLSCAASGFTFSNYDMNWVRQAPGKGLEWISY
IYGSSRDISYADFVKGRFTISRDNAKNSLYLQMNSLRAEDTAVYYCVRSS
GYYFGYGMDVWGRGTLVTVSS

In one embodiment, the anti-CD38 antibody or antigen-binding fragment thereof according to the present invention is named “Clone D07” according to the Examples below, and comprises a HCVR with a sequence set forth in SEQ ID NO: 37; or a variant thereof.

SEQ ID NO: 37
EVQLVESGGSLVKPGGSLRLSCAASGFTFSNSAMNWVRQAPGKGLEWISY
ISGSSRDISYADFVKGRFTISRDNAKNSLYLQMNSLRAEDTAVYYCVRSS
SSYYYSGSGMDVWGRGTLVTVSS

In one embodiment, the anti-CD38 antibody or antigen-binding fragment thereof according to the present invention is named “Clone D10” according to the Examples below, and comprises a HCVR with a sequence set forth in SEQ ID NO: 38; or a variant thereof.

SEQ ID NO: 38
EVQLVESGGSLVKPGGSLRLSCAASGFTFSNSAMNWVRQAPGKGLEWISS
ISGSSRDIYYADFVKGRFTISRDNAKNSLYLQMNSLRAEDTAVYYCVRSS
SSYYYSGNGMDVWGRGTLVTVSS

In one embodiment, the anti-CD38 antibody or antigen-binding fragment thereof according to the present invention is named “Clone B08” according to the Examples below, and comprises a LCVR with a sequence as set forth in SEQ ID NO: 39; or a variant thereof.

SEQ ID NO: 39
QSVLTQPASVSGSPGQSITISCAGTSSDVGGSYSVSWYQQHPGKAPKLMI
YDDSNRPSGVSNRFSGSKSGNTASLTISGLQAEDEADYYCSSYTDYSTRV
FGGGTKL

In one embodiment, the anti-CD38 antibody or antigen-binding fragment thereof according to the present invention is named “Clone C05” according to the Examples below, and comprises a LCVR with a sequence as set forth in SEQ ID NO: 40; or a variant thereof.

In one embodiment, the anti-CD38 antibody or antigen-binding fragment thereof according to the present invention is named “Clone C06” according to the Examples below, and comprises a LCVR with a sequence as set forth in SEQ ID NO: 40; or a variant thereof.

SEQ ID NO: 40
QSVLTQPASVSGSPGQSITISCAGTSSDVGGSSYVSWYQQHPGKAPKLM
IYYDSYRPSGVSNRFSGSKSGNTASLTISGLQAEDEADYYCSSNTYYST
RVFGGGTKL

In one embodiment, the anti-CD38 antibody or antigen-binding fragment thereof according to the present invention is named “Clone D06” according to the Examples below, and comprises a LCVR with a sequence set forth in SEQ ID NO: 41; or a variant thereof.

SEQ ID NO: 41
QSVLTQPASVSGSPGQSITISCAGTSSDVGGSYYVSWYQQHPGKAPKLMI
YSDSYRPSGVSNRFSGSKSGNTASLTISGLQAEDEADYYCSSNTAYSTRV
FGGGTKL

In one embodiment, the anti-CD38 antibody or antigen-binding fragment thereof according to the present invention is named “Clone D07” according to the Examples below, and comprises a LCVR with a sequence set forth in SEQ ID NO: 42; or a variant thereof.

In one embodiment, the anti-CD38 antibody or antigen-binding fragment thereof according to the present invention is named “Clone D10” according to the Examples below, and comprises a LCVR with a sequence set forth in SEQ ID NO: 42; or a variant thereof.

SEQ ID NO: 42
QSVLTQPASVSGSPGQSITISCAGTSSDVGGNSYVSWYQQHPGKAPKLMI
YYDSYRPSGVSNRFSGSKSGNTASLTISGLQAEDEADYYCSSNTYYSTRV
FGGGTKL

In one embodiment, the anti-CD38 antibody or antigen-binding fragment thereof according to the present invention comprises:
a HCVR as defined hereinabove; and
a LCVR as defined hereinabove.

In one embodiment, the anti-CD38 antibody or antigen-binding fragment thereof according to the present invention comprises:
a HCVR selected from SEQ ID NOs: 33, 34, 35, 36, 37 and 38; and
a LCVR selected from SEQ ID NOs: 39, 40, 41 and 42.

In one embodiment, the anti-CD38 antibody or antigen-binding fragment thereof according to the present invention is named “Clone B08” according to the Examples below, and comprises:
a HCVR with a sequence as set forth in SEQ ID NO: 33; and
a LCVR with a sequence as set forth in SEQ ID NO: 39.

In one embodiment, the anti-CD38 antibody or antigen-binding fragment thereof according to the present invention is named “Clone C05” according to the Examples below, and comprises:
a HCVR with a sequence as set forth in SEQ ID NO: 34; and
a LCVR with a sequence as set forth in SEQ ID NO: 40.

In one embodiment, the anti-CD38 antibody or antigen-binding fragment thereof according to the present invention is named “Clone C06” according to the Examples below, and comprises:
a HCVR with a sequence as set forth in SEQ ID NO: 35; and
a LCVR with a sequence as set forth in SEQ ID NO: 40.

In one embodiment, the anti-CD38 antibody or antigen-binding fragment thereof according to the present invention is named “Clone D06” according to the Examples below, and comprises:
a HCVR with a sequence as set forth in SEQ ID NO: 36; and
a LCVR with a sequence as set forth in SEQ ID NO: 41.

In one embodiment, the anti-CD38 antibody or antigen-binding fragment thereof according to the present invention is named “Clone D07” according to the Examples below, and comprises:
a HCVR with a sequence as set forth in SEQ ID NO: 37; and
a LCVR with a sequence as set forth in SEQ ID NO: 42.

In one embodiment, the anti-CD38 antibody or antigen-binding fragment thereof according to the present invention is named “Clone D10” according to the Examples below, and comprises:
a HCVR with a sequence as set forth in SEQ ID NO: 38; and
a LCVR with a sequence as set forth in SEQ ID NO: 42.

A variant of a HCVR and/or of a LCVR refers to any of the HCVR and/or LCVR as defined hereinabove characterized as having an amino acid sequence that shares at least 75%, 80%, 85%, 90%, 95%, 96%, 97%, 98%, 99% or more of identity with the particular HCVR and/or LCVR.

A variant of a HCVR and/or of a LCVR refers to any of the HCVR and/or LCVR as defined hereinabove characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids being substituted by a different amino acid.

In one embodiment, the anti-CD38 antibody or antigen-binding fragment thereof according to the present invention has no significant affinity and hence has no significant ability to bind to CD3. In one embodiment, the anti-CD38 antibody or antigen-binding fragment thereof according to the present invention is not an anti-CD3/anti-CD38 bispecific antibody. Illustratively, anti-CD3/anti-CD38 bispecific antibodies are disclosed in WO2016071355.

In one embodiment, the anti-CD38 antibody or antigen-binding fragment thereof according to the present invention is chimeric.

In one embodiment, a chimeric anti-CD38 antibody or antigen-binding fragment thereof according to the present invention comprises a heavy chain and/or a light chain, comprising a human constant region and a variable region from another species, such as, e.g., a murine variable region.

In one embodiment, the anti-CD38 antibody or antigen-binding fragment thereof according to the present invention is humanized.

In one embodiment, the anti-CD38 antibody or antigen-binding fragment thereof according to the present invention is humanized, preferably is a humanized monoclonal antibody or antigen-binding fragment, more preferably comprises:
a light chain constant region (LCCR) derived from a human κ LCCR, and
a heavy chain constant region (HCCR) derived from a human IgG1, IgG2, IgG3 or IgG4 (wild type or mutated) HCCR.

In one embodiment, the anti-CD38 antibody or antigen-binding fragment thereof according to the present invention is humanized, preferably is a humanized monoclonal antibody or antigen-binding fragment, more preferably comprises:
a light chain constant region (LCCR) derived from a human κ LCCR, and
a heavy chain constant region (HCCR) derived from a human IgG1 comprising a D297A or a D297G mutation or derived from a human IgG4 comprising a S228P mutation.

The humanized anti-CD38 antibody or antigen-binding fragment thereof according to the present invention has the advantage to be less immunogenic (or completely non-immunogenic) than murine versions when administrated to human subjects.

As well known by one skilled in the art, the choice of IgG isotypes of the HCCR centers on whether specific functions are required and the need for a suitable in vivo half-life. For example, antibodies designed for selective eradication of cancer cells typically require an active isotype that permits complement activation and effector-mediated cell killing by antibody-dependent cell-mediated cytotoxicity. Both human IgG1 and IgG3 (shorter half-life) isotypes meet these criteria, particularly human IgG1 isotype (wild type and variants). In particular, depending of the IgG isotype of the HCCR (particularly human wild type and variants IgG1 isotype), the antibody can be cytotoxic towards cells via a CDC, ADCC and/or ADCP mechanism (Salfeld, 2007. Nat Biotechnol. 25(12):1369-72; Irani et al., 2015. Mol Immunol. 67(2 Pt A):171-82). In fact, the fragment crystallizable (Fc) region interacts with a variety of accessory molecules to mediate indirect effector functions such as antibody-dependent cellular cytotoxicity (ADCC), antibody-dependent cellular phagocytosis (ADCP) and complement-dependent cytotoxicity (CDC).

In one embodiment, the anti-CD38 antibody or antigen-binding fragment thereof according to the present invention can be modified to enhance ADCC, ADCP and/or CDC. Such modifications are well known in the art.

For example, antibodies comprising a low fucose content are known to enhance ADCC response via the FcγRIII receptor (see, e.g., International patent publication WO2014140322). Thus, the antibody according to the present invention may comprise a low fucose content.

The term “fucose content”, as used herein, refers to the percentage of fucosylated forms within the N-glycans attached to the N297 residue of the Fc fragment of each heavy chain of each antibody.

The term “low fucose content”, as used herein, refers to a fucose content of less than, or equal to, 65%. Advantageously, the fucose content is less than or equal to 65%, preferably less than or equal to 60%, 55% or 50%, or even less than or equal to 45%, 40%, 35%, 30%, 25% or 20%. However, it is not necessary that the fucose content be zero, and it may for example be greater than or equal to 5%, 35 10%, 15% or 20%.

In one embodiment, the anti-CD38 antibody or antigen-binding fragment thereof according to the present invention may further comprise different types of glycosylation (N-glycans of the oligomannose or biantennary complex type, with a variable proportion of bisecting N-acetylglucosamine (GlcNAc) residues or galactose residues in the case of N-glycans of the biantennary complex type), provided that they have a low fucose content (see, e.g., International patent publication WO2007048077). For example, antibodies having slightly fucosylated N-glycans can be obtained as described in European patent publication EP1176195 or in International patent publications WO2001077181 or WO2012041768.

The N-glycans of the oligomannose type have reduced half-life in vivo as compared to N-glycans of the biantennary complex type. Consequently, advantageously, the anti-CD38 antibody or antigen-binding fragment thereof according to the present invention has on its N-glycosylation sites of the Fc fragment glycan structures of the biantennary complex type, with a low fucose content, as defined above.

In one embodiment, the anti-CD38 antibody or antigen-binding fragment thereof according to the present invention is modified to facilitate delivery across the blood-brain barrier (BBB). Means and methods to modify antibodies to facilitate their crossing through the BBB, e.g., when parentally administered, are well known in the art, and are described in, e.g., Yu et al., 2011. Sci Transl Med. 3(84):84ra44; Atwal et al., 2011. Sci Transl Med. 3(84):84ra43; and International patent applications WO2015031673 and WO2016208695.

In some embodiments, the anti-CD38 antibody or antigen-binding fragment thereof according to the present invention is conjugated to a therapeutic moiety, i.e., a drug. In one embodiment, the therapeutic moiety is selected from a cytotoxin, a chemotherapeutic agent, a cytokine, an immunosuppressant, an immune stimulator, a lytic peptide and a radioisotope. Such conjugates are referred to herein as an “antibody drug conjugates” or “ADCs”.

In one embodiment, the compound according to the present invention is an isolated nucleic acid encoding an anti-CD38 antibody or an antigen-binding fragment thereof as described hereinabove.

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably more than 96%, 97%, 98% or 99% by weight; or
(2) homogeneity as shown by agarose gel electrophoresis and using an intercalating agent such as ethidium bromide, SYBR Green, GelGreen or the like.

It will be readily understood that the one skilled in the art can design nucleic acid sequences encoding any of the HCVRs and/or LCVRs disclosed herein.

It is further understood that the one skilled in the art is familiar with molecular biology methods aiming at modifying a nucleic acid sequence in order to improve, e.g., recombinant production rates, such as by codon optimization. Ultimately, the present application encompasses any nucleic acid encoding any HCVRs and/or LCVRs as disclosed herein.

In one embodiment, the compound according to the present invention is an expression vector comprising the isolated nucleic acid encoding an anti-CD38 antibody or an antigen-binding fragment thereof as described hereinabove.

In one embodiment, the expression vector according to the present invention comprises a sequence encoding the HCVR of the antibody or binding fragment thereof according to the invention, operably linked to regulatory elements.

In one embodiment, the expression vector according to the present invention comprises a sequence encoding the LCVR of the antibody or binding fragment thereof according to the invention, operably linked to regulatory elements.

In one embodiment, the expression vector according to the present invention comprises:
a sequence encoding the HCVR of the antibody or binding fragment thereof according to the invention, operably linked to regulatory elements, and
a sequence encoding the LCVR of the antibody or binding fragment thereof according to the invention, operably linked to regulatory elements.

In one embodiment, the compound according to the present invention is an antigen-binding antibody mimetic.

All the embodiments disclosed herein for antibodies or antigen-binding fragment thereof are transposed mutatis mutandis to the antigen-binding antibody mimetics according to the present invention.

In one embodiment, the compound according to the present invention is a small organic molecule.

The present invention also relates to a composition comprising or consisting of or consisting essentially of the compound which specifically competes with CD31 for CD38 binding according to the present invention.

The present invention also relates to a pharmaceutical composition comprising the compound which specifically competes with CD31 for CD38 binding according to the present invention; and at least one pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, e.g., FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The present invention also relates to a medicament comprising or consisting of or consisting essentially of the compound which specifically competes with CD31 for CD38 binding according to the present invention. The present invention further relates to the use of a compound which specifically competes with CD31 for CD38 binding as disclosed herein for the preparation or the manufacture of a medicament.

The present invention also relates to methods of preventing and/or treating a disease in a subject in need thereof, comprising administering to said subject the compound which specifically competes with CD31 for CD38 binding, the composition, the pharmaceutical composition or the medicament according to the present invention.

The present invention also relates to the compound which specifically competes with CD31 for CD38 binding, the composition, the pharmaceutical composition or the medicament according to the present invention, for use as a drug. The present invention also relates to the compound which specifically competes with CD31 for CD38 binding, the composition, the pharmaceutical composition or the medicament according to the present invention, for use in preventing and/or treating a disease in a subject in need thereof.

In one embodiment, the disease is related to, due to or caused by increased soluble CD38 levels in the subject. In particular, increased soluble CD38 levels may be detected in a blood sample (including whole blood, plasma and serum, preferably in a plasma sample), in a cerebrospinal fluid sample and/or in a muscle biopsy.

In one embodiment, the disease is selected from the group comprising or consisting of: neurodegenerative diseases; neuroinflammatory diseases; inflammatory diseases; autoimmune diseases; metabolic diseases; ocular diseases; age-related diseases and aging; and cancer and metastasis. In one embodiment, the disease is selected from the group comprising, or consisting of, a neurodegenerative disease, a neuroinflammatory disease, an inflammatory disease, an autoimmune disease and a combination thereof.

The present disclosure also encompasses symptoms of the recited diseases.

Examples of neurodegenerative diseases include, but are not limited to, Parkinson's disease and related disorders including Parkinson's disease, Parkinson-dementia, autosomal recessive PARK2 and PARK6-linked Parkinsonism, atypical parkinsonian syndromes, including, progressive supranuclear palsy, corticobasal degeneration syndrome, Lewy bodies dementia, multiple system atrophy, Guadeloupean Parkinsonism and Lytigo-bodig disease; motor neuron diseases including amyotrophic lateral sclerosis, frontotemporal dementia, progressive bulbar palsy, pseudobulbar palsy, primary lateral sclerosis, progressive muscular atrophy, spinal muscular atrophy and post-polio syndrome; neuro-inflammatory diseases; Alzheimer's disease and related disorders including early stage of an Alzheimer's disorder, mild stage of an Alzheimer's disorder, moderate stage of an Alzheimer's disorder, mild to moderate stage of an Alzheimer's disorder, advanced stage of an Alzheimer's disorder, mild cognitive impairment, vascular dementia, mixed dementia, Pick's disease, argyrophilic grain disease, posterior cortical atrophy, Wernicke-Korsakoff Syndrome; prion diseases; lysosomal storage diseases; leukodystrophies; Huntington's Disease; multiple sclerosis; Down syndrome; spinal and bulbar muscular atrophy; HIV-Associated Neurocognitive Disorder; Tourette Syndrome; autosomal dominant spinocerebellar ataxia; Friedreich's Ataxia; Dentatorubral pallidoluysian atrophy; myotonic dystrophy; schizophrenia; age associated memory impairment; autism and autism spectrum disorders; attention-deficit hyperactivity disorder; chronic pain; alcohol-induced dementia; progressive non-fluent aphasia; semantic dementia; spastic paraplegia; fibromyalgia; post-Lyme disease; neuropathies; withdrawal symptoms; Alpers' disease; cerebro-oculo-facio-skeletal syndrome; Wilson's disease; Cockayne syndrome; Leigh's disease; neurodegeneration with brain iron accumulation; opsoclonus myoclonus syndrome; alpha-methylacyl-CoA racemase deficiency; Andermann syndrome; Arts syndrome; Marinesco-Sjögren syndrome; mitochondrial membrane protein-associated neurodegeneration; pantothenate kinase-associated neurodegeneration; polycystic lipomembranous osteodysplasia with sclerosing leukoencephalopathy; riboflavin transporter deficiency neuronopathy; and ataxia telangiectasia.

In one embodiment, the invention relates to the anti-CD38 antibodies or antigen-binding fragments thereof named “Clone B08” or “Clone D06” according to the present invention for use in preventing and/or treating a neurodegenerative disease in a subject in need thereof.

The terms “neuroinflammatory disease” and “inflammatory demyelinating disease” can be used interchangeably, to refer to a disease in which there is inflammation of one or more areas of the brain or the spinal cord. The terms are not fully equivalent, however, and the one skilled in the art will appreciate that a neuroinflammatory disease does not necessarily requires demyelination, although demyelination often develops following the inflammation.

Examples of neuroinflammatory diseases include, but are not limited to, multiple sclerosis; acute disseminated encephalomyelitis; optic neuritis; transverse myelitis; neuromyelitis optica; and the like. In some instances, primary conditions with secondary neuroinflammation (e.g., traumatic brain injury with secondary neuroinflammation) may be considered a neuroinflammatory disease as it relates to the subject disclosure.

In one embodiment, the invention relates to the anti-CD38 antibody or antigen-binding fragment thereof named “Clone B08” according to the present invention for use in preventing and/or treating a neuroinflammatory disease in a subject in need thereof.

Examples of inflammatory diseases include, but are not limited to, arthritis (including osteoarthritis, rheumatoid arthritis, spondyloarthropathies and psoriatic arthritis); asthma (including atopic asthma, nonatopic asthma, allergic asthma, exercise-induced asthma, drug-induced asthma, occupational asthma and late stage asthma); inflammatory bowel disease (including Crohn's Disease, ulcerative colitis and colitis); inflammatory skin disorders (including psoriasis, atopic dermatitis and contact hypersensitivity); multiple sclerosis; osteoporosis; tendonitis; allergic disorders (including rhinitis, conjunctivitis and urticaria); inflammation in response to an insult to the host (including injury or infection); transplant rejection; graft versus host disease; sepsis; and systematic lupus erythematosus.

In one embodiment, the invention relates to the anti-CD38 antibodies or antigen-binding fragments thereof named “Clone B08”, “Clone C05”, “Clone D06” or “Clone D07” according to the present invention for use in preventing and/or treating an inflammatory disease in a subject in need thereof.

Examples of autoimmune diseases include, but are not limited to, alopecia areata; ankylosing spondylitis; arthritis; antiphospholipid syndrome; autoimmune Addison's disease; autoimmune hemolytic anemia; autoimmune inner ear disease (also known as Meniere's disease); autimmune lymphoproliferative syndrome; autoimmune thrombocytopenic purpura; autoimmune hemolytic anemia; autoimmune hepatitis; Bechet's disease; Crohn's disease; diabetes mellitus type 1; glomerulonephritis; Graves' disease; Guillain-Barré syndrome; inflammatory bowel disease; lupus nephritis; multiple sclerosis; myasthenis gravis; pemphigus; pernicous anemia; polyarteritis nodosa; polymyositis; primary biliary cirrhosis; psoriasis; Raynaud's phenomenon; rheumatic fever; rheumatoid arthritis; scleroderma; Sjögren's syndrome; systemic lupus erythematosus; ulcerative colitis; vitiligo; and Wegener's granulamatosis.

In one embodiment, the invention relates to the anti-CD38 antibodies or antigen-binding fragments thereof named “Clone B08”, “Clone C05”, “Clone D06” or “Clone D07” according to the present invention for use in preventing and/or treating an autoimmune disease in a subject in need thereof.

Examples of metabolic diseases include, but are not limited to, diabetes (e.g., type 1 diabetes, type 2 diabetes, gestational diabetes); hyperglycemia; insulin resistance; impaired glucose tolerance; hyperinsulinism; diabetic complication; dyslipidemia; hypercholesterolemia; hypertriglyceridemia; HDL hypocholesterolemia; LDL hypercholesterolemia and/or HLD non-cholesterolemia; VLDL hyperproteinemia; dyslipoproteinemia; apolipoprotein A-1 hypoproteinemia; metabolic syndrome; syndrome X; obesity; non-alcoholic fatty liver disease (NAFLD); nonalcoholic steatohepatitis; and adrenal leukodystrophy.

The term “cancer” as used herein is meant to encompass primary and secondary cancers, as well as recurrent, metastatic and/or multi-drug resistant cancers.

Examples of cancer include, but are not limited to, breast cancer; prostate cancer; lung cancer; ovarian cancer; colorectal cancer; brain cancer. Other non-limiting examples of cancers include biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Still other examples of cancers include lymphomas, sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, synovioma, mesothelioma, lymphangioendotheliosarcoma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia and heavy chain.

Examples of ocular diseases include, but are not limited to, age-related macular degeneration, glaucoma, macular edema, diabetic retinopathy, uveitis, allergic conjunctivitis, scleritis, keratitis, keratoconjunctivitis, vernal keratoconjunctivitis, atopic keratoconjunctivitis, cicatrizing conjunctivitis, blepharitis, enophthalmitis, retinitis, retinopathies, choroiditis, Sjogren's syndrome and retinal necrosis.

“Aging”, also referred to as “organismal aging”, is manifested by age-related diseases, the incidence of which increases with age. Death from aging means death from age-related diseases. Suppression or alleviation of aging delays one, some or most age-related diseases. Slow aging is manifested by delayed age-related diseases. Slow aging is considered to be a type healthy aging.

Examples of age-related diseases include, but are not limited to, benign tumors; cardiovascular diseases, such as stroke, atherosclerosis and hypertension; angioma; osteoporosis; insulin-resistance and type II diabetes, including diabetic retinopathy and neuropathy; Alzheimer's disease; Parkinson's disease; age-related macular degeneration; arthritis; seborrheic keratosis; actinic keratosis; photoaged skin; skin spots; skin cancer; systemic lupus erythematosus; psoriasis; smooth muscle cell proliferation and intimal thickening following vascular injury; inflammation; arthritis; side effects of chemotherapy; benign prostatic hyperplasia; as well as less common diseases wherein their incidence is higher in elderly people than in young people.

In one embodiment, the disease is selected from the group comprising or consisting of amyotrophic lateral sclerosis; Parkinson's disease and related disorders; Alzheimer's disease and related disorders; Huntington disease; multiple sclerosis; rheumatoid arthritis; systemic lupus erythematosus; diabetes; obesity; non-alcoholic steatohepatitis; age-related macular degeneration; and glaucoma. In one embodiment, the disease is selected from the group comprising or consisting of amyotrophic lateral sclerosis; Parkinson's disease and related disorders; Alzheimer's disease and related disorders; and multiple sclerosis. In some embodiments, the disease is amyotrophic lateral sclerosis. In some embodiments, the disease is multiple sclerosis. In some embodiments, the disease is Parkinson's disease and related disorders. In some embodiments, the disease is Alzheimer's disease and related disorders.

In one embodiment, the methods according to the present invention are for delaying organismal aging.

In one embodiment, the compound which specifically competes with CD31 for CD38 binding, the composition, the pharmaceutical composition or the medicament according to the present invention is for use in delaying organismal aging.

The present invention also relates to methods of increasing the level of at least one anti-inflammatory cytokine in a subject in need thereof, in particular in the blood of said subject, comprising administering to said subject the compound which specifically competes with CD31 for CD38 binding, the composition, the pharmaceutical composition or the medicament according to the present invention.

The present invention also relates to the compound which specifically competes with CD31 for CD38 binding, the composition, the pharmaceutical composition or the medicament according to the present invention, for use in increasing the level of at least one anti-inflammatory cytokine in a subject in need thereof, in particular in the blood of said subject.

Examples of anti-inflammatory cytokines include, but are not limited to, interleukin-10 (IL-10), transforming growth factor ß (TGF-ß), interleukin-1ra (IL-1ra), interleukin-4 (IL-4), interleukin-6 (IL-6), interleukin-11 (IL-11), interleukin-13 (IL-13) and interleukin-22 (IL-22).

Methods to measure blood cytokine levels in a subject are known in the art. Such methods include, for example, the use of ELISA test, such as the one described in the Example section below.

In particular, the present invention relates to a method of increasing the level of IL-10 in a subject in need thereof, in particular in the blood of said subject, comprising administering to said subject the compound which specifically competes with CD31 for CD38 binding, the composition, the pharmaceutical composition or the medicament according to the present invention.

In particular, the present invention relates to the compound which specifically competes with CD31 for CD38 binding, the composition, the pharmaceutical composition or the medicament according to the present invention, for use in increasing the level of IL-10 in a subject in need thereof, in particular in the blood of said subject.

Methods to measure IL-10 levels in a blood sample from a subject are well known in the art and include, e.g., the use of an ELISA test.

In one embodiment, the compound which specifically competes with CD31 for CD38 binding, the composition, the pharmaceutical composition or the medicament according to the present invention triggers an increase of IL-10 levels in the blood of a subject by at least 100%, 200%, 300%, in particular at least 400% and more particularly at least 500%, as compared to the administration of negative control molecule.

In one embodiment, the subject is an animal. In one embodiment, the subject is a mammal.

Examples of mammals include, but are not limited to, primates (including human and non-human), cattle (including cows), horses, pigs, sheep, goats, dogs and cats.

In a preferred embodiment, the subject is a human.

In one embodiment, the subject is an adult (e.g., a subject above the age of 18 in human years or a subject after reproductive capacity has been attained). In another embodiment, the subject is a child (for example, a subject below the age of 18 in human years or a subject before reproductive capacity has been attained).

In one embodiment, the subject is a male. In one embodiment, the subject is a female.

In one embodiment, the subject is/was diagnosed with a disease selected from the group comprising or consisting of neurodegenerative diseases; neuroinflammatory diseases; inflammatory diseases; autoimmune diseases; metabolic diseases; ocular diseases; age-related diseases; and cancer and metastasis.

In one embodiment, the subject is/was diagnosed with a disease selected from the group comprising or consisting of amyotrophic lateral sclerosis; Parkinson's disease and related disorders; Alzheimer's disease and related disorders; Huntington disease; multiple sclerosis; rheumatoid arthritis; systemic lupus erythematosus; diabetes; obesity; non-alcoholic steatohepatitis; age-related macular degeneration; and glaucoma.

In one embodiment, the subject is at risk of developing a disease selected from the group comprising or consisting of neurodegenerative diseases; neuroinflammatory diseases; inflammatory diseases; autoimmune diseases; metabolic diseases; ocular diseases; age-related diseases; and cancer and metastasis.

In one embodiment, the subject is at risk of developing a disease selected from the group comprising or consisting of amyotrophic lateral sclerosis; Parkinson's disease and related disorders; Alzheimer's disease and related disorders; Huntington disease; multiple sclerosis; rheumatoid arthritis; systemic lupus erythematosus; diabetes; obesity; non-alcoholic steatohepatitis; age-related macular degeneration; and glaucoma.

In one embodiment, the subject is/was diagnosed with increased plasma soluble CD38 levels.

In one embodiment, the subject is/was diagnosed with increased cerebrospinal fluid soluble CD38 levels.

In one embodiment, the subject is/was diagnosed with increased soluble CD38 levels in a muscle biopsy.

Methods to detect and measure soluble CD38 levels are well known to the one skilled in the art. In particular, means and kits for detecting and measuring soluble CD38 levels are commercially available.

In one embodiment, the compound, composition, pharmaceutical composition or medicament according to the present invention will be formulated for administration to the subject.

In one embodiment, the compound, composition, pharmaceutical composition or medicament according to the present invention is to be administered systemically or locally.

In one embodiment, the compound, composition, pharmaceutical composition or medicament according to the present invention is to be administered by injection, orally, topically, nasally, buccally, rectally, vaginaly, intratracheally, by endoscopy, transmucosally, or by percutaneous administration.

In one embodiment, the compound, composition, pharmaceutical composition or medicament according to the present invention is to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv), subcutaneous, intramuscular (im), intradermal (id), intraperitoneal (ip), perispinal injection and perfusion.

In one embodiment, when injected, the compound, composition, pharmaceutical composition or medicament according to the present invention is sterile. Methods for obtaining a sterile composition include, but are not limited to, GMP synthesis (where GMP stands for “Good manufacturing practice”).

Sterile injectable forms of a composition may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It will be understood that other suitable routes of administration are also contemplated in the present invention, and the administration mode will ultimately be decided by the attending physician within the scope of sound medical judgment. Apart from administration by injection (iv, ip, im and the like), other routes are available, such as nebulization (Respaud et al., 2014. MAbs. 6(5):1347-55; Guilleminault et al., 2014. J Control Release. 196:344-54; Respaud et al., 2015. Expert Opin Drug Deliv. 12(6):1027-39) or subcutaneous administration (Jackisch et al., 2014. Geburtshilfe Frauenheilkd. 74(4):343-349; Solal-Celigny, 2015. Expert Rev Hematol. 8(2):147-53).

In one embodiment, the compound, composition, pharmaceutical composition or medicament according to the present invention is able to cross the blood-brain barrier (BBB), preferably when using an intravenous or subcutaneous route of administration.

Methods to deliver compounds, compositions, pharmaceutical compositions or medicaments when injected intravenously across the BBB are known in the art, in particular when said compounds are antibodies. Indeed, several publications demonstrated that intravenous injection of monoclonal antibodies effectively crossed the blood-brain barrier (BBB). Pharmacokinetic modeling of brain concentration of intravenously injected monoclonal antibody demonstrated that around 0.4% of plasma mAb concentrations are found in the brain (Shah & Bets, 2013. MAbs. 5(2):297-305). Brain/plasma ratio of Aducanumab and ABBV-8E12 were found to reach 1.3% and 0.385% in humans, respectively (Sevigny et al. 2016. Nature. 537(7618):50-6; West et al., 2017. J Prev Alz Dis. 4(4):236-241). Moreover, several strategies have been developed to improve BBB crossing including the use of bispecific antibodies targeting insulin receptor or transferrin receptor (for review see Neves et al., 2016. Trends Biotech. 34(1):36-48) or using ultrasound-induced BBB opening, as described in Konofagou et al., 2012. Curr Pharm Biotechnol. 13(7): 1332-45.

In one embodiment, the compound, composition, pharmaceutical composition or medicament according to the present invention is to be administered to the subject in need thereof in a therapeutically effective amount.

Such therapeutically amount can be determined by one skilled in the art by routine tests including assessment of the effect of administration of the compound, composition, pharmaceutical composition or medicament on the diseases which are sought to be prevented and/or to be treated by the administration of said the compound, composition, pharmaceutical composition or medicament according to the invention.

For example, such tests can be implemented by analyzing both quantitative and qualitative effect of the administration of different amounts of said aforementioned compound, composition, pharmaceutical composition or medicament according to the present invention on a set of markers (biological and/or clinical) characteristics of said diseases, in particular from a biological sample of a subject.

It will be however understood that the total daily usage of the compound, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the compound, composition, pharmaceutical composition or medicament employed; the age, body weight, general health, sex and diet of the subject; the time of administration, route of administration, and rate of excretion of the compound, composition, pharmaceutical composition or medicament employed; the duration of the treatment; drugs used in combination or coincidental with the compound, composition, pharmaceutical composition or medicament employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, a therapeutically effective amount of the compound, composition, pharmaceutical composition or medicament according to the present invention ranges from about 0.1 mg/kg to about 50 mg/kg, from about 0.5 mg/kg to about 45 mg/kg, from about 1 mg/kg to about 40 mg/kg, from about 2.5 mg/kg to about 35 mg/kg, from about 5 mg/kg to about 30 mg/kg, from about 10 mg/kg to about 20 mg/kg. In one embodiment, a therapeutically effective amount of the compound, composition, pharmaceutical composition or medicament according to the present invention is about 15 mg/kg.

In one embodiment, a therapeutically effective amount of the compound, composition, pharmaceutical composition or medicament according to the present invention ranges from about 10 μg/kg to about 4 mg/kg (human equivalent dose (HED)), from about 40 μg/kg to about 3.6 mg/kg (HED), from about 80 μg/kg to about 3.2 mg/kg (HED), from about 200 μg/kg to about 2.8 mg/kg (HED), from about 400 μg/kg to about 2.4 mg/kg (HED), from about 800 μg/kg to about 2 mg/kg (HED). In one embodiment, a therapeutically effective amount of the compound, composition, pharmaceutical composition or medicament according to the present invention is about 1.2 mg/kg (HED).

In one embodiment, a therapeutically effective amount of the compound, composition, pharmaceutical composition or medicament according to the present invention is to be administered once a day, twice a day, three times a day or more.

In one embodiment, a therapeutically effective amount of the compound, composition, pharmaceutical composition or medicament according to the present invention is to be administered every day, every two days, every three days, every four days, every five days, every six days.

In one embodiment, a therapeutically effective amount of the compound, composition, pharmaceutical composition or medicament according to the present invention is to be administered every week, every two weeks, every three weeks.

In one embodiment, a therapeutically effective amount of the compound, composition, pharmaceutical composition or medicament according to the present invention is to be administered every month, every two months, every three months, every four months, every five months, every six months.

In a preferred embodiment, a therapeutically effective amount of the compound, composition, pharmaceutical composition or medicament according to the present invention is to be administered every 12 hours, every 24 hours, every 36 hours, every 48 hours, every 60 hours, every 72 hours, every 96 hours.

In a preferred embodiment, a therapeutically effective amount of the compound, composition, pharmaceutical composition or medicament according to the present invention is to be administered every 60 hours.

In one embodiment, the compound, composition, pharmaceutical composition or medicament according to the present invention is for acute administration. In one embodiment, the compound, composition, pharmaceutical composition or medicament according to the present invention is for chronic administration.

In one embodiment, a therapeutically effective amount of the compound, composition, pharmaceutical composition or medicament according to the present invention is to be administered for about 5 days, 7 days, 10 days, 14 days, 21 days, 28 days, 1 month, 2 months, 3 months, 6 months, 1 year or more.

In one embodiment, the compound, composition, pharmaceutical composition or medicament according to the present invention is to be administered before, concomitantly with or after a therapeutic drug.

Some examples of therapeutic drugs suitable for co-administration with the compound, composition, pharmaceutical composition or medicament according to the present invention include, without limitation, immunosuppressants, cytotoxins, chemotherapeutic agents, cytokines, immune stimulators, lytic peptides and radioisotopes.

It will be understood by the one skilled in the art that the co-administration of the compound, composition, pharmaceutical composition or medicament according to the present invention with a particular therapeutic drug, which may be chosen among those recited herein but without being limited thereto, will depend on the disease or condition to be prevented and/or treated.

Examples of immunosuppressants include, without limitation, mTOR inhibitors such as, e.g., sirolimus, everolimus, ridaforolimus, temsirolimus, umirolimus and zotarolimus; IL-1 receptor antagonists such as, e.g., anakinra; antimetabolites such as, e.g., azathioprine, leflunomide, methotrexate, mycophenolic acid and teriflunomide; IMiDs such as, e.g., apremilast, lenalidomide, pomalidomide and thalidomide; and antibodies such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, abatacept, belatacept, etanercept, pegsunercept, aflibercept, alefacept and rilonacept.

Examples of cytotoxins include, without limitation, radionuclides (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, and ²¹¹At), conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others.

Examples of chemotherapeutic agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan or topotecan); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, cyclophosphamide, chlorambucil, carmustine or lomustine; anti-tumor anthracycline derivatives (suc has, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene); aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); kinase inhibitors (such as, e.g., flavoperidol, imatinib mesylate or gefitinib); farnesyltransferase inhibitors; and HDAC inhibitors.

Examples of cytokines include, without limitation, chemokines (such as, e.g., CCL1, CCL2/MCP1, CCL3/MIP1α, CCL4/MIP1β, CCL5/RANTES, CCL6, CCL7, CCL8, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, CXCL1/KC, CXCL2, CXCL3, CXCL4, CXCL5, CXCL6, CXCL7, CXCL8/IL8, CXCL9, CXCL10, CXCL11, CXCL12, CXCL13, CXCL14, CXCL15, CXCL16, CXCL17, CX3CL1, XCL1 and XCL2), tumor necrosis factors (such as, e.g., TNFA, Lymphotoxin, TNFSF4, TNFSF5/CD40LG, TNFSF6, TNFSF7, TNFSF8, TNFSF9, TNFSF10, TNFSF11, TNFSF13, TNFSF13B and EDA) and interleukins (such as, e.g., IL-1α, IL-1β, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, IFNα, IFNβ, IFNκ, IFNω and GM-CSF).

Examples of immune stimulators include, without limitation, filgrastim, pegfilgrastim, lenograstim, molgramostim, sargramostim, ancestim, albinterferon, interferon alfa, peginterferon alfa, interferon beta, peginterferon beta, interferon gamma, aldesleukin, oprelvekin, growth hormone, immunocyanin, pegademase, prolactin, tasonermin, histamine dihydrochloride, poly ICLC, vitamin D, lentinan, plerixafor, roquinimex, mifamurtide, glatiramer acetate, thymopentin, thymosin al, thymulin, polyinosinic:polycytidylic acid, pidotimod, Bacillus Calmette-Guérin vaccine, melanoma vaccine and sipuleucel-T vaccine.

Examples of lytic peptides include, without limitation, toxins (such as, e.g., Diptheria toxin or Pseudomonas exotoxin).

Examples of radioisotopes include, without limitation, the radionuclides of technetium (e.g., Tc-99 and Tc-97), potassium (e.g., K-40), rubidium (e.g., Rb-82), iodine (e.g., I-123, I-124, I-125, I-129, I-131), cesium (e.g., Cs-135, Cs-137), cobalt (e.g., Co-60), palladium (e.g., Pd-103, Pd-107), cadmium (e.g., Cd-113), strontium (e.g., Sr-89, Sr-90), europium (e.g., Eu-55), tin (e.g., Sn-121, Sn-126), phosphorus (e.g., P-32, P-33), thallium (e.g., T1-201), indium (e.g., In-111), gallium (e.g., Ga-67, Ga-68), yttrium (e.g., Y-90), iridium (e.g., Ir-192), bismuth (e.g., Bi-213), radium (e.g., Ra-223, Ra-225), and ruthenium (e.g., Ru-106).

The present invention also relates to a method of manufacturing a compound according to the present invention, which comprises a step of selecting a compound which specifically competes with CD31, as detailed hereinabove.

In one embodiment where the compound is an antibody, an antigen-binding fragment thereof or an antigen-binding antibody mimetic, the method comprises or consists of selecting an antibody, an antigen-binding fragment thereof or an antigen-binding antibody mimetic which has a K_{D} value inferior or equal to 10⁻⁷ M, preferably inferior or equal to 10⁻⁸, more preferably inferior or equal to 10⁻⁹ M for CD38, even more preferably inferior or equal to 1.10⁻¹⁰ M, as may be determined by biosensor analysis, particularly by Biacore Analysis.

In one embodiment where the compound is a small organic molecule, the method comprises or consists of selecting a small organic molecule which displaces CD31 binding with a K_{D} value inferior or equal to 10⁻⁶ M, preferably inferior or equal to 10⁻⁷ M for CD38, more preferably inferior or equal to 1.10⁻⁸ M, as may be determined by biosensor analysis, particularly by Biacore Analysis.

In one embodiment where the compound is an oligonucleotide, the method comprises or consists of selecting an oligonucleotide which displaces CD31 binding with a K_{D} value inferior or equal to 200 nM, preferably inferior or equal to 150 nM for CD38, more preferably inferior or equal to 100 nM, as may be determined by biosensor analysis, particularly by Biacore Analysis.

As above indicated, the capacity of a compound to compete with CD31 can be tested by FACS or Biacore analysis.

In one embodiment where the compound is an antibody, an antigen-binding fragment thereof or an antigen-binding antibody mimetic, the method of manufacturing a compound according to the present invention may further comprise a step of selecting an antibody, an antigen-binding fragment thereof or an antigen-binding antibody mimetic which specifically binds to at least one epitope within CD38, as defined herein above.

Preferably, the step of selecting an antibody, an antigen-binding fragment thereof or an antigen-binding antibody mimetic which specifically binds to at least one epitope within CD38 comprises selecting an antibody, an antigen-binding fragment thereof or an antigen-binding antibody mimetic which specifically binds:
to at least one epitope extending at least throughout amino acid residue 220 to amino acid residue 285 of human CD38 with SEQ ID NO: 2; and/or
to cysteine 254 and/or 275 of human CD38 with SEQ ID NO: 2; and/or
to the 5^{th} C-terminal disulfide loop involving cysteine 254 and cysteine 275 of human CD38 with SEQ ID NO: 2.

In one embodiment where the compound is an antibody, an antigen-binding fragment thereof or an antigen-binding antibody mimetic, the method of manufacturing a compound according to the present invention may further comprise a step of selecting an antibody, an antigen-binding fragment thereof or an antigen-binding antibody mimetic which does not mediate, trigger or enhance ADCC, ADCP and/or CDC.

In one embodiment, the method comprises or consists of selecting a compound having at least one, preferably at least two, more preferably the three following properties:
it protects dopaminergic neurons against energy deficit due to mitochondrial complex I inhibition, in particular against the mitochondrial neurotoxin MPP⁺;
it increases the release of the anti-inflammatory cytokine interleukin-10 (IL-10) in vitro using human peripheral blood mononuclear cells (PBMC); and/or
it increases IL-10 levels in vivo when injected to mice.

In one embodiment, the method comprises or consists of selecting a compound which competes with CD31, preferably with human CD31, and induces tyrosine phosphorylation. In particular, the method comprises or consists of selecting a compound which induces tyrosine phosphorylation of discrete cytoplasmic substrates that is inhibited in the presence of genistein.

In one embodiment, the method comprises or consists of selecting a compound which competes with CD31, preferably with human CD31, and triggers CD38 internalization.

In one embodiment, the method comprises or consists of selecting a compound which competes with CD31, preferably with human CD31, and induces lysosomal exocytosis. In particular, the method comprises or consists of selecting a compound which induces lysosomal exocytosis that is inhibited in the presence of vacuolin-1.

In one aspect, the invention also relates to a method for identifying individuals in need of treatment against a disease selected in the group comprising or consisting of neurodegenerative diseases, neuroinflammatory diseases, inflammatory diseases, autoimmune diseases, metabolic diseases, ocular diseases, age-related diseases and aging, and cancer and metastasis, that are susceptible to respond to an efficient amount of a compound according to the invention, in particular of an anti-CD38 antibody or antigen-binding fragment thereof according to the invention, the method comprising the measure of the CD38 level.

The invention further relates to the use of the measure of the CD38 level in an individual in need of treatment against a disease selected in the group comprising or consisting of neurodegenerative diseases, neuroinflammatory diseases, inflammatory diseases, autoimmune diseases, metabolic diseases, ocular diseases, age-related diseases and aging, and cancer and metastasis, as a biomarker, in particular to determine whether said individual is susceptible to respond to an efficient amount of a compound according to the invention, in particular to an efficient amount of an anti-CD38 antibody or antigen-binding fragment thereof according to the invention.

In one embodiment, the CD38 level is the plasma CD38 level. In one embodiment, the CD38 level is the cerebrospinal fluid CD38 level. Methods for measuring CD38 levels are well known in the state of the art, and may include an ELISA assay, with an adapted anti-CD38 antibody. Suitable antibodies for ELISA may be purchased, e.g., from R&D SYSTEMS®.

In some embodiment, the method further comprises the step of comparing the CD38 level measured in the said individual with a reference value. In one embodiment, the reference value may represent the CD38 level in a healthy individual or a group of healthy individuals. As used herein, “healthy individual(s)” refers to individual(s) not having any of the above-mentioned diseases. In one embodiment, a statistic significant increase in the CD38 level measured in the said individual as compared to the reference value may indicate that the individual is susceptible to respond to the anti-CD38 antibody or antigen-binding fragment thereof according to the invention in the treatment of the disease.

### BRIEF DESCRIPTION OF DRAWINGS

FIG. 1 is a plot showing the neuroprotective effect of sCD31 against MPP⁺-induced dopaminergic (DA) cell death. DA (TH⁺) cell survival in midbrain cultures treated with MPP⁺ (3 μM) between 5 and 7 DIV exposed or not to sCD31 (0.01, 0.03, 0.1, 0.3, 1 μg/ml). Results are expressed in % of corresponding control cultures. $ P<0.05 one-way ANOVA vs control treatment; #P<0.05 one-way ANOVA vs MPP⁺ treatment.

FIG. 2 is a bar graph showing the neuroprotective effect of sCD31 against MPP⁺-induced DA cell death. DA (TH⁺) cell survival in midbrain cultures treated with MPP⁺ (3 μM) between 5 and 7 DIV exposed or not to sCD31 (1 μg/ml) in the presence or not of anti-CD31 antibody clone Moon-1 (1 μg/ml), of the antagonistic anti-CD38 antibody clone OKT10 (OKT, 1 μg/ml), or of the antagonistic anti-CD38 antibody clone AT13/5 (1 μg/ml). Results are expressed in % of corresponding control cultures. $ P<0.05 one-way ANOVA vs control treatment; #P<0.05 one-way ANOVA vs MPP⁺ treatment.

FIG. 3 is a bar graph showing the neuroprotective effect of agonistic anti-CD38 antibody clone HB7 against MPP⁺-induced DA cell death. DA (TH⁺) cell survival in midbrain cultures treated with MPP⁺ (3 μM) between 5 and 7 DIV exposed or not to agonistic anti-CD38 antibody (clone HB7, 1 μg/ml) in the presence or not of the antagonistic anti-CD38 antibody clone OKT10 (OKT, 1 μg/ml), or of the antagonistic anti-CD38 antibody clone AT13/5 (1 μg/ml). Results are expressed in % of corresponding control cultures. $ P<0.05 one-way ANOVA vs control treatment; #P<0.05 one-way ANOVA vs MPP treatment.

FIG. 4 is a bar graph showing the neuroprotective effect of sCD31 or agonistic anti-CD38 antibody clone HB7 against MPP⁺-induced DA cell death in the presence or absence of the tyrosine kinase inhibitor genistein. DA (TH⁺) cell survival in midbrain cultures treated with MPP (3 μM) between 5 and 7 DIV exposed or not to sCD31 (1 μg/ml) or agonistic anti-CD38 antibody (clone HB7, 1 μg/ml) in the presence or not of genistein (GENI, 10 nM), an inhibitor of tyrosine kinase. Results are expressed in % of corresponding control cultures. $ P<0.05 one-way ANOVA vs control treatment; #P<0.05 one-way ANOVA vs MPP treatment.

FIG. 5 is a bar graph showing the neuroprotective effect of sCD31 or agonistic anti-CD38 antibody clone HB7 against MPP⁺-induced DA cell death in the presence or absence of the lysosomal exocytosis inhibitor vacuolin-1 or endosidin2. DA (TH⁺) cell survival in midbrain cultures treated with MPP (3 μM) between 5 and 7 DIV exposed or not to sCD31 (1 μg/ml) or agonistic anti-CD38 antibody (clone HB7, 1 μg/ml) in the presence or not of vacuolin-1 (VAC, 10 μM), or endosidin2 (40 μM), two inhibitors of lysosomal maturation and exocytosis. Results are expressed in % of corresponding control cultures. $ P<0.05 one-way ANOVA vs control treatment; #P<0.05 one-way ANOVA vs MPP treatment.

FIG. 6 is a bar graph showing the effect of agonistic anti-CD38 HB7 antibody injected intravenously (15 mg/kg iv, two days before MPTP injections) in the in vivo MPTP mouse model on the number of DA (TH⁺) neurons in the substantia nigra pars compacta. Results are expressed in % of control (PBS-injected) mice. PBS group: n=5; MPTP group: n=6; MPTP+HB7 15 mg/kg iv group: n=8. $ P<0.05 one-way ANOVA vs control (PBS-injected) group. #P<0.05 one-way ANOVA vs MPTP group.

FIG. 7 is a bar graph showing the effect of intravenous injection of agonistic anti-CD38 clone HB7 on IL-10 levels in mice. $ P<0.05 one-way ANOVA vs control (PBS-injected) group.

FIG. 8 is a diagram depicting the process used to identify anti-CD38 antibodies that competes with CD31.

FIG. 9 is a bar graph showing the neuroprotective effect of new humanized anti-CD38 antibodies (clone A02, B06, B08, B09, C05, C06, C08, D03, D04, D06, D07, D10, E03, E05, E08, F07) identified by phage display or anti-CD38 antibody clone HB7 against MPP⁺-induced DA cell death. DA (TH⁺) cell survival in midbrain cultures treated with MPP (3 μM) between 5 and 7 DIV exposed or not to anti-CD38 antibodies (clone A02, B06, B08, B09, C05, C06, C08, D03, D04, D06, D07, D10, E03, E05, E08, F07; 0.5 μg/ml) or anti-CD38 antibody clone HB7 (0.5 μg/ml). Results are expressed in % of corresponding control cultures. #P<0.05 one-way ANOVA vs MPP⁺ treatment.

FIG. 10 is a bar graph showing the neuroprotective effect of new humanized anti-CD38 antibodies (clones B08, C05, C06, D06 and D07) identified by phage display against MPP⁺-induced DA cell death in the presence or absence of the lysosomal exocytosis inhibitor vacuolin-1. DA (TH⁺) cell survival in midbrain cultures treated with MPP⁺ (3 μM) between 5 and 7 DIV exposed or not to anti-CD38 antibodies (clones B08, C05, C06, C08, D06 and D07; 1 μg/ml) in the presence or not of vacuolin-1 (VAC, 10 μM), an inhibitor of lysosomal exocytosis. Results are expressed in % of corresponding control cultures. $ P<0.05 one-way ANOVA vs control treatment; #P<0.05 one-way ANOVA vs MPP⁺ treatment.

FIG. 11 is a bar graph showing the effect of anti-CD38 antibodies (clone HB7, B08 or D06) injected intracerebrally (0.1 mg/kg icy, concomitantly with 6-OHDA icy injection) in the in vivo 6-OHDA mouse model on the number of DA (TH⁺) neurons in the substantia nigra pars compacta. Results are expressed in % of contralateral (non-lesioned) side. PBS group: n=8 (PBS); 6-OHDA+PBS group: n=8 (PBS); 6-OHDA+HB7 0.1 mg/kg icy group: n=10 (HB7 icy 0.1 mg/kg); 6-OHDA+B08 0.1 mg/kg icy group: n=7 (B8 icy 0.1 mg/kg); 6-OHDA+D06 0.1 mg/kg icy group: n=7 (D6 icy 0.1 mg/kg). $ P<0.05 one-way ANOVA vs control (PBS-injected) group. #P<0.05 one-way ANOVA vs 6-OHDA group.

FIG. 12 is a bar graph showing the effect of anti-CD38 antibodies (clone B08 or Daratumumab, DARA) injected intravenously (15 mg/kg iv, one day before 6-OHDA icy injection) in the in vivo 6-OHDA mouse model on the number of DA (TH⁺) neurons in the substantia nigra pars compacta. Results are expressed in % of contralateral (non-lesioned) side. PBS group: n=8 (PBS); 6-OHDA group: n=8 (PBS); 6-OHDA+B08 15 mg/kg iv group: n=10 (B8 iv 15 mg/kg); 6-OHDA+Daratumumab 15 mg/kg iv group: n=10 (DARA iv 15 mg/kg). $ P<0.05 one-way ANOVA vs control (PBS-injected) group. #P<0.05 one-way ANOVA vs 6-OHDA group.

FIG. 13 is a bar graph showing the effect of the B08 anti-CD38 antibody injected intravenously (15 mg/kg iv, two days after 6-OHDA icy injection) in the in vivo 6-OHDA mouse model on the number of DA (TH⁺) neurons in the substantia nigra pars compacta. Results are expressed in % of contralateral (non-lesioned) side. PBS group: n=12 (PBS); 6-OHDA+PBS group: n=12 (PBS); 6-OHDA+B08 15 mg/kg iv group: n=12 (B8 iv 15 mg/kg). $ P<0.05 one-way ANOVA vs control (PBS-injected) group. #P<0.05 one-way ANOVA vs 6-OHDA group.

FIG. 14 is a bar graph showing the effect of anti-CD38 antibodies (clone HB7, B08, C05, D06 or D07) on the release of the anti-inflammatory cytokine IL-10 using human peripheral blood mononuclear cells from 3 healthy donors (pooled results). $ P<0.05 one-way ANOVA vs control (PBS-treated) group.

FIG. 15A-B is a set of scheme and graph. (A) is a scheme representing the protocol. (B) is a bar graph showing the effect of anti-CD38 antibody (clone B08) injected intravenously (15 mg/kg iv, 10 and 20 days after MOG injection) in the in vivo EAE mouse model on the clinical score. PBS group: n=10 (PBS); B08 15 mg/kg iv group: n=10 (B8 15 mg/kg iv). * P<0.05 two-way ANOVA vs PBS group. ** P<0.01 two-way ANOVA vs PBS group. Clinical score is calculated as described in example 9, section 2.4b).

FIG. 16A-B is a set of schema and graph. (A) is a scheme representing the protocol. (B) is a bar graph showing the effect of anti-CD38 antibodies (clone B08 or Daratumumab, DARA) injected intravenously (15 mg/kg iv, injected at D0) in the in vivo DSS mouse model on colon length. No-DSS group: n=5 (PBS; white bar); DSS+PBS group: n=6 (PBS; black bar); DSS+DARA 15 mg/kg iv group: n=7 (DARA iv 15 mg/kg; black bar); DSS+B08 15 mg/kg iv group: n=6 (B8 iv 15 mg/kg; black bar). $ P<0.05 one-way ANOVA vs No-DSS group. #P<0.05 one-way ANOVA vs DSS group.

FIG. 17 is a bar graph showing plasma CD38 levels in human healthy controls (HC) or patients with amyotrophic lateral sclerosis (ALS). HC group: n=46; ALS group: n=40. *** P<0.001, t-test vs HC group.

### EXAMPLES

The present invention is further described in the following examples, but the technical scope of the present invention is not limited to these examples.

### Example 1: In Vitro Neuroprotection of Midbrain Dopaminergic Neurons by sCD31 is Mediated Through Interaction with CD38

Protection Against the Mitochondrial Neurotoxin MPP⁺

We report here that sCD31 is neuroprotective in a situation in which DA cell death was caused by mitochondrial poisoning with 1-methyl-4-phenylpyridinium (MPP⁺), the active metabolite of the DA neurotoxin 1-methyl-4-phenyl-1,2,3,6-tetrahydropyridine (MPTP).

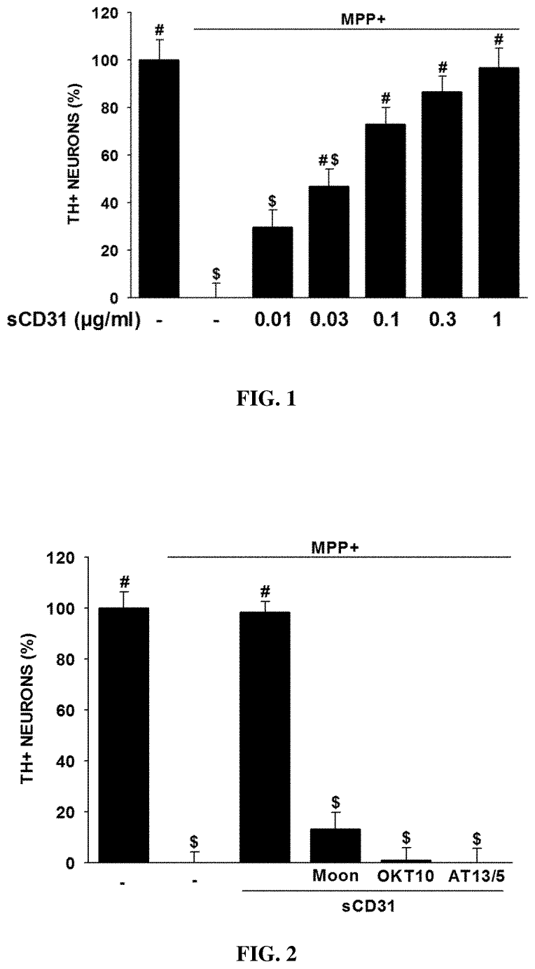

For this purpose, spontaneously occurring DA cell death was prevented by a treatment combining depolarizing concentrations of K⁺ (30 mM) and MK801 (5 μM), a glutamate receptor antagonist used to prevent unwanted excitotoxic insult. The cultures were then exposed to 3 μM MPP between 5 and 7 days in vitro (DIV) to achieve a loss of approximately 50% of DA neurons. When the cultures were exposed to murine sCD31 (with SEQ ID NO: 3) during the intoxication period, MPP⁺-induced DA cell loss was prevented by this compound (FIG. 1) with an EC₅₀ of 36 ng/mL.

Of note, these effects are true, direct neuroprotective effects, and are not due to a possible interference with CD38 localized on immune cells (as illustrated in Hara-Yokoyama et al., 2008. Int Immunopharmacol. 8(1):59-70) since there are no peripheral immune cells in this in vitro cell culture setting.

The Neuroprotective Effect of sCD31 is Antagonized in the Presence of Anti-CD31 Antibody Clone Moon-1 or Antagonistic Anti-CD38 Antibodies (Clone OKT-10 or AT13/5)

CD31 is known to interact with 3 ligands: CD31 itself, CD38 and the αᵥβ₃ integrin (Kalinowska & Losy, 2006. Eur J Neurol. 13(12):1284-90). However, CD31 is not expressed in neuronal cells (Williams et al., 1996. J Neurosci Res. 45(6):747-57). Interaction of CD31 with CD38 was previously shown to be antagonized in the presence of the anti-CD31 antibody clone Moon-1 or by antagonistic anti-CD38 antibodies (Deaglio et al., 1996. J Immunol. 156(2):727-34; Deaglio et al., 1998. J Immunol. 160(1):395-402).

To test whether the neuroprotective effect of sCD31 is mediated through CD38, we studied the neuroprotective effect of sCD31 (1 μg/mL) in the presence or the absence of anti-CD31 antibody clone Moon-1 (1 μg/mL) or antagonistic anti-CD38 antibodies (clone OKT10 or AT13/5, 1 μg/mL) in the previously described MPP⁺ in vitro model.

We observed that the neuroprotective effect of sCD31 was antagonized in the presence of anti-CD31 antibody clone Moon-1 or of the antagonistic anti-CD38 antibodies clones OKT10 or AT13/5, thus meaning that the neuroprotective effect of sCD31 is mediated through the interaction with CD38 (FIG. 2).

The Neuroprotective Effect of sCD31 is Reproduced by Agonistic Anti-CD38 Antibody (Clone HB7)

Agonistic anti-CD38 antibodies are compounds that reproduce the biological effect of CD31 binding to CD38 (Deaglio et al., 1998. J Immunol. 160(1):395-402). Thus, like sCD31, agonistic anti-CD38 antibodies should also be neuroprotective.

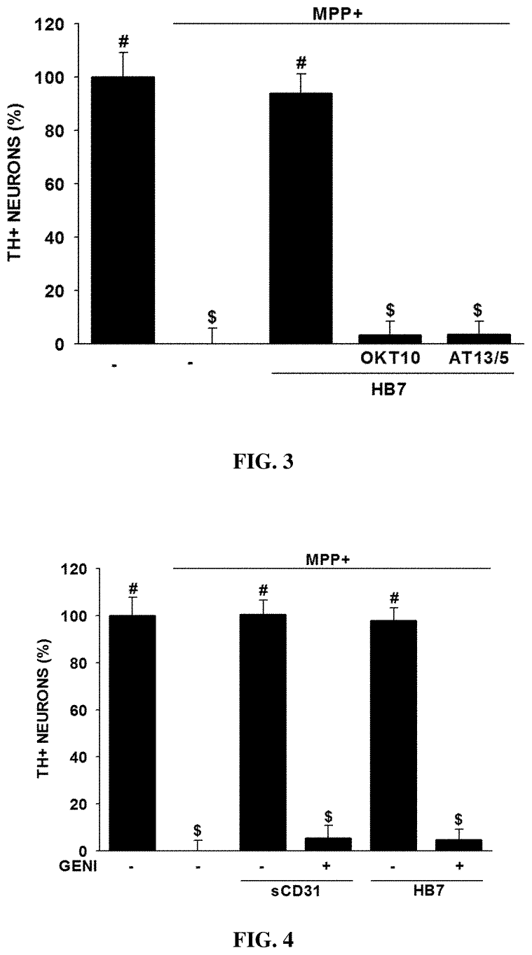

We observed that an agonistic anti-CD38 antibody (clone HB7, 1 μg/mL) was able to protect DA neurons in the MPP in vitro model, and that this neuroprotective effect was antagonized in the presence of antagonistic anti-CD38 antibodies (clone OKT10 or AT13/5, 1 μg/mL) (FIG. 3).

The Neuroprotective Effect of sCD31 or of Agonistic Anti-CD38 Antibodies Requires Tyrosine Phosphorylation/Activation of Tyrosine Kinase

As previously mentioned herein, CD38 is a multifunctional molecule including a receptor-mediated function through internalization, a tyrosine phosphorylation-mediated function and an enzyme-mediated function. Previous research demonstrated that agonistic (clone HB7) or antagonistic (clone OKT10 or AT13/5) anti-CD38 antibodies failed to modulate CD38 enzymatic activity in vitro (Deckert et al., 2014. Clin Cancer Res. 20(17):4574-83). Consequently, we wanted to determine whether sCD31 or agonistic anti-CD38 antibodies required tyrosine phosphorylation to mediate its neuroprotective effect, by using genistein, an inhibitor of tyrosine kinase.

We observed that, in the MPP in vitro model, the neuroprotective effect of sCD31 (1 μg/mL) or of agonistic anti-CD38 antibodies (clone HB7, 1 μg/mL) was antagonized in the presence of the tyrosine kinase inhibitor genistein (GENI, 10 nM), suggesting that tyrosine phosphorylation is necessary to protect DA neurons (FIG. 4).

The Neuroprotective Effect of sCD31 or of Agonistic Anti-CD38 Antibodies is Mediated Through Lysosomal Exocytosis

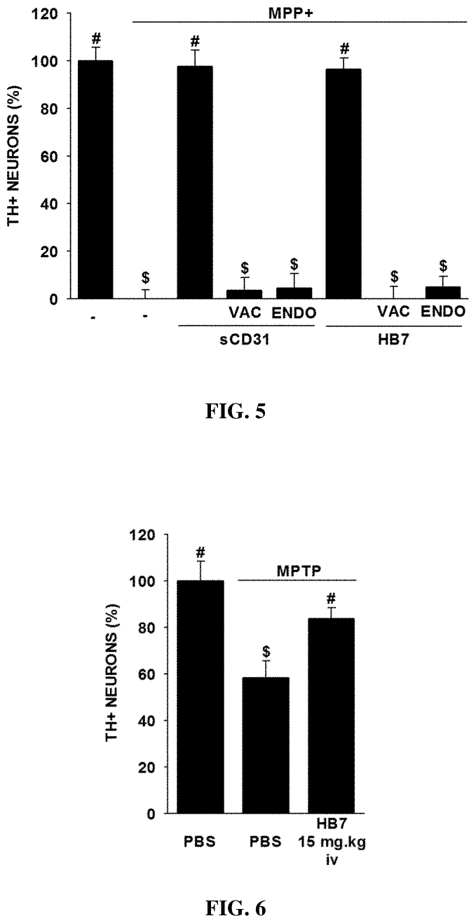

One may assume that sCD31 and agonistic anti-CD38 antibodies could both protect dopaminergic neurons by acting at the CD38 level but through a different mechanism of action. Ruling out this possibility, we observed (FIG. 5) that in the MPP in vitro model, the neuroprotective effect of sCD31 (1 μg/mL) or of agonistic anti-CD38 antibody (clone HB7, 1 μg/mL) were both antagonized in the presence of the inhibitors of lysosomal maturation and of Ca²⁺-dependent lysosomal exocytosis vacuolin-1 and endosidin2.

Agonistic Anti-CD38 Antibody (Clone HB7) is Neuroprotective In Vivo

To determine whether agonistic anti-CD38 antibody (clone HB7) was also neuroprotective in vivo, we intravenously injected a 15 mg/kg dose of this antibody in a mouse model in which dopaminergic neurons of the substantia nigra pars compacta degenerate following repeated intraperitoneal injection of MPTP. MPTP has the ability to cross the blood brain barrier and is converted into MPP by the astrocytes. MPP⁺, which is an inhibitor of the mitochondrial complex I, is preferentially transported in dopaminergic neurons through the dopamine transporter, resulting in a specific cell death of dopaminergic population. This model has been extensively used as a test system for assessment of neuroprotective and neurorepair strategies (Dauer & Przedborski, 2003. Neuron. 39(6):889-909).

We observed that intravenous injection of agonistic anti-CD38 antibody (clone HB7) at a 15 mg/kg dose strongly protected dopaminergic neurons of the substantia nigra pars compacta from MPTP-induced neurodegeneration (FIG. 6).

Conclusion

Altogether, the data presented in this example show that sCD31 is neuroprotective in vitro, an effect which is mediated through the interaction with CD38. This effect is antagonized in the presence of anti-CD31 antibodies or antagonistic anti-CD38 antibodies.

Additionally, it has been demonstrated that agonistic anti-CD38 antibodies, which mimic the binding of sCD31 to CD38, are also able to induce a neuroprotective effect, said effect being antagonized in the presence of antagonistic anti-CD38 antibodies.

Whether the neuroprotective effect is induced by sCD31 or by agonistic anti-CD38 antibodies, tyrosine phosphorylation/activation of tyrosine kinase has been shown to be necessary to induce said neuroprotective effect. Moreover, the resulting neuroprotective effect has been shown to be due to lysosomal maturation and Ca²⁺-dependent lysosomal exocytosis.

Finally, agonistic anti-CD38 antibodies have been shown to be neuroprotective in vivo in the widely-used MPTP mouse model using an intravenous route of administration, thus demonstrating that agonistic anti-CD38 antibodies are able to cross the BBB and engage their target.

These data therefore suggest a therapeutic role of sCD31 and agonistic anti-CD38 antibodies for the treatment of neurodegenerative and neuroinflammatory diseases.

### Example 2: In Vivo Effect of sCD31 or Agonistic Anti-CD38 Antibodies (Clone HB7) on IL-10 Levels

Agonistic Anti-CD38 Antibody (Clone HB7) Increases IL-10 In Vivo

To test whether the anti-inflammatory properties of agonistic anti-CD38 antibodies were also observe in vivo, we intravenously injected anti-CD38 antibody clone HB7 (15 mg/kg) to mice.

Eight days following injection, we observed a 6-fold increase in IL-10 levels (FIG. 7).

Conclusion

Altogether, the data presented in this example show that both sCD31 and agonistic anti-CD38 antibodies induce a strong release of IL-10 in vivo, suggesting a role in immunoregulation and against inflammation. Indeed, IL-10 is widely known to downregulate the expression of Tₕ1 cytokines, MHC class II antigens, and co-stimulatory molecules on macrophages; to enhance B cell survival, proliferation, and antibody production; to block NF-κB activity; and to regulate the JAK-STAT signaling pathway.

Moreover, IL-10 has also been shown to be effective in treating cancer, in particular by inhibiting tumor metastasis (Sun et al., 2000. J Immunother. 23(2):208-14). Expression of IL-10 from transfected tumor cell lines in IL-10 transgenic mice (Groux et al., 1999. J Immunol. 162(3):1723-9) or dosing with IL-10 leads to control of primary tumor growth and decreased metastatic burden (Fujii et al., 2001. Blood. 98(7):2143-51; Berman et al., 1996. J Immunol. 157(1):231-8).

These data therefore suggest a therapeutic role of sCD31 and agonistic anti-CD38 antibodies for the treatment of inflammatory diseases and cancer.

### Example 3: Identification of Agonistic Anti-CD38 Antibodies that Competes with CD31 by Phage Display

To generate new humanized antibodies that reproduce the biological effect of CD31 on CD38, we used a phage display library of scFvs containing 1.5 billion sequences (Philibert et al., 2007. BMC Biotechnol. 7:81).

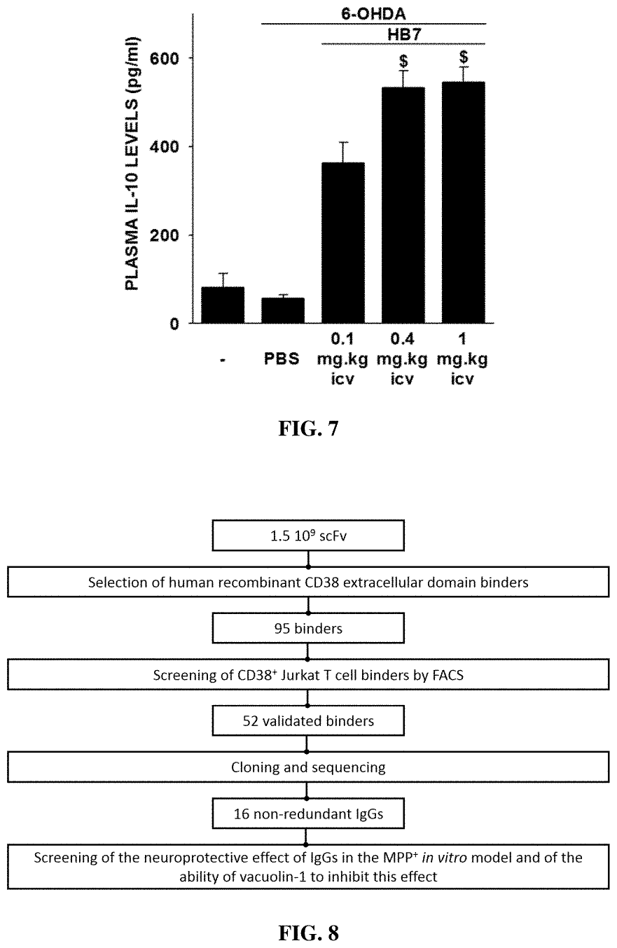

A first round of selection was done to identify scFvs that bind human CD38 extracellular domain (FIG. 8). Among 95 selected scFvs, 52 were confirmed to bind to CD38+Jurkat T cells by FACS. These 52 validated binders were sequenced, leading to the identification of 16 non-redundant scFvs that bind CD38. These binders were cloned and the corresponding IgG were produced in CHO cells.

The 16 non-redundant IgGs were then tested (clones A02, B06, B08, B09, C05, C06, C08, D03, D04, D06, D07, D10, E03, E05, E08, F07), first in the MPP in vitro model (at 0.5 μg/mL for each clone), then by competition assay with the human extracellular domain of CD31 by Biacore on a panel of selected antibodies.

In the MPP in vitro model, several humanized anti-CD38 antibodies clones were found to be neuroprotective (FIG. 9). In particular, 4 clones (B08, C05, D06 and D10) shows a stronger neuroprotective effect than that of the reference agonistic anti-CD38 antibody clone HB7 (0.5 μg/mL).

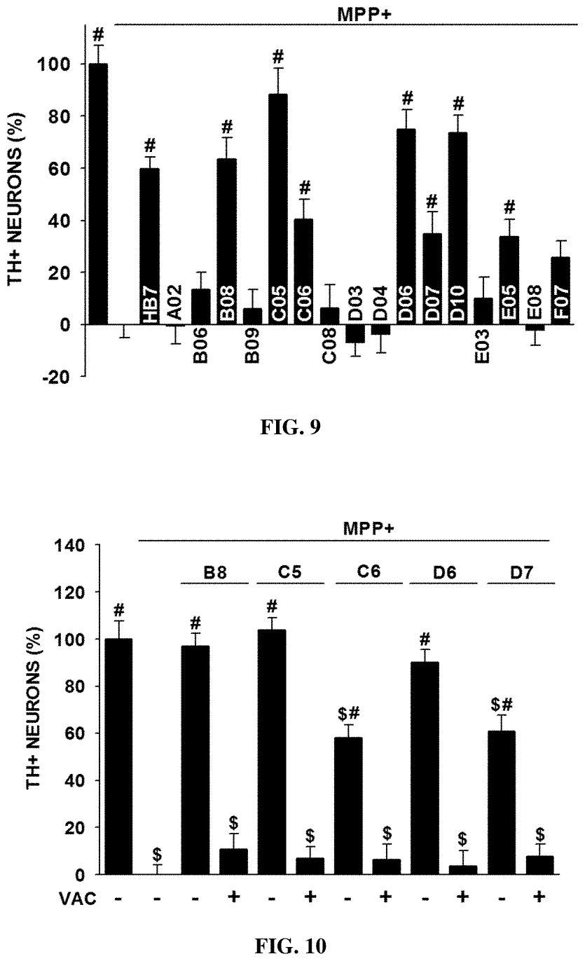

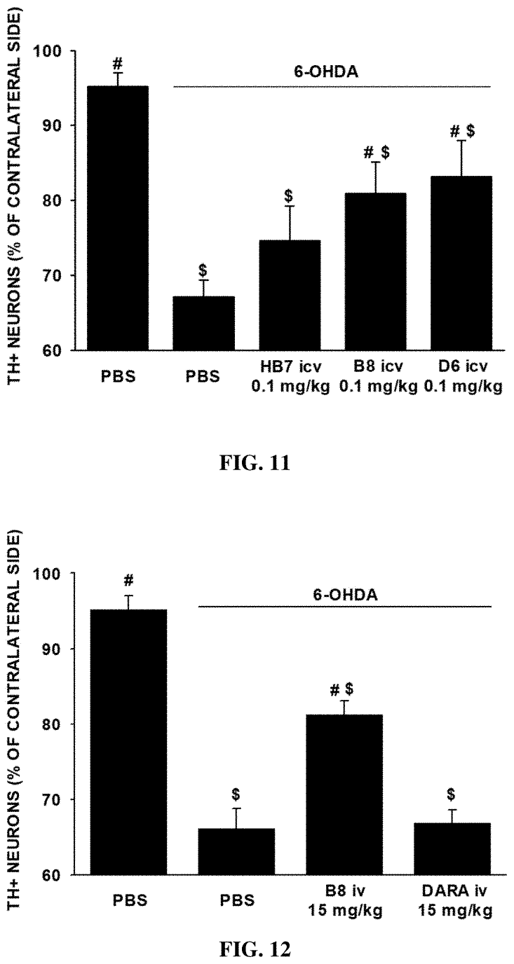

The neuroprotective effect of clones B08, C05, C06, D06 and D07 was antagonized in the presence of the lysosomal exocytosis inhibitors vacuolin-1 (VAC, 10 μM), suggesting that these antibodies reproduce the biological effect of sCD31 (FIG. 10).

### Example 4: Neuroprotective Properties of B08 and C06 Anti-CD38 Antibodies in an In Vivo Neurodegenerative Model

We aimed at determining whether B08 or D06 anti-CD38 antibodies were neuroprotective in vivo against neurodegeneration induced by oxidative stress. Mouse were administered with 6-hydroxydopamine (6-OHDA) in the right striatum. Since 6-OHDA is an analogue of dopamine, it is transported in dopaminergic neurons through the dopamine transporter, resulting in a specific degeneration and loss of dopaminergic neurons of the right substantia nigra pars compacta. It has been extensively used as a test system for novel symptomatic agents and for assessment of neuroprotective and neurorepair strategies (Galindo et al., 2014. In Kostrzewa (Ed.), Handbook of neurotoxicity (1^{st} Ed., pp. 639-651). New York: Springer-Verlag). Since 6-OHDA is injected unilaterally in the right striatum, left striatum remains unaffected and each mouse is its own control.

Mice were intracerebrally injected with 0.1 mg/kg of HB7, B08 and D06 anti-CD38 antibodies. We observed that at a concentration of 0.1 mg/kg injected intracerebrally (icy) concomitantly with 6-OHDA, B08 and D06 anti-CD38 antibodies statistically significantly protected dopaminergic neurons of the right substantia nigra pars compacta (FIG. 11). We also observed that HB7 anti-CD38 antibody did fail to statistically significantly protect dopaminergic neurons.

Of interest, regarding B08 anti-CD38 antibody, this neuroprotective effect was also observed when injected intravenously (iv, 15 mg/kg) one day before 6-OHDA icy injection (FIG. 12). Noticeably, the anti-CD38 antibody Daratumumab (DARA, 15 mg/kg iv) failed to demonstrate neuroprotective activity.

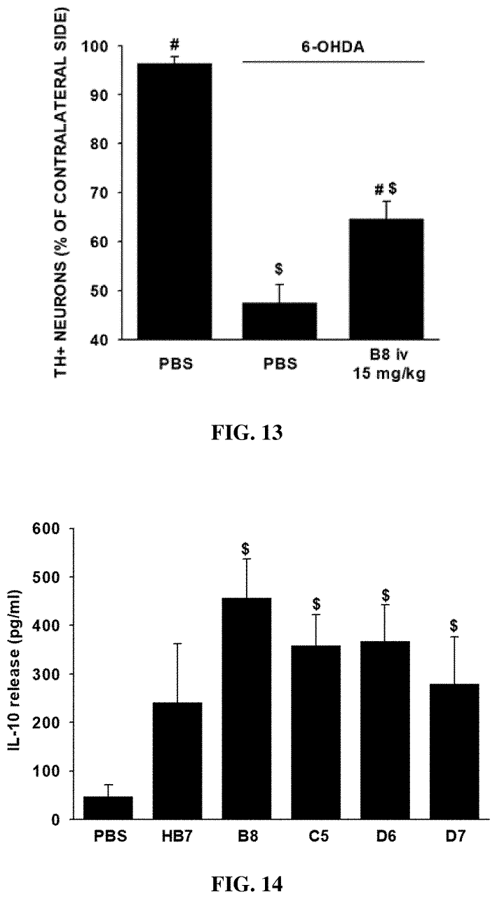

To understand whether B08 anti-CD38 antibody could also protect neurons when the neurodegenerative process is ongoing, we injected B08 anti-CD38 antibody (at 15 mg/kg iv) two days after 6-OHDA icy injection. We still observed strong neuroprotective activity of the B08 anti-CD38 antibody even after degeneration has started (FIG. 13). This demonstrates the powerful neuroprotective effect of B08 anti-CD38 antibody.

As a conclusion, the neuroprotective effect of B08 and D06 anti-CD38 antibodies were demonstrated to be of superior efficacy than that obtained with HB7 anti-CD38 antibody. Noticeably, this neuroprotective effect is still observed when the compound is injected intravenously when the neurodegenerative process is ongoing.

### Example 5: Anti-Inflammatory Properties of the B08, C05, D06 and D07 Anti-CD38 Antibodies In Vitro

To determine whether agonistic anti-CD38 antibodies (clone B08, C05, D06 and D07) possess anti-inflammatory properties, we assessed their effect on the release of the anti-inflammatory cytokine IL-10 in vitro using human peripheral blood mononuclear cells (PBMCs) from 3 healthy donors. We observed that anti-CD38 antibodies (clone B08, C05, D06 and D07) all statistically significantly increased IL-10 release, thus demonstrating anti-inflammatory properties (FIG. 14). Of importance, HB7 failed to statistically significantly increase IL-10 release in culture human PBMCs in this assay, demonstrating the higher anti-inflammatory effect of the B08, C05, D06 and D07 anti-CD38 antibodies over the HB7 anti-CD38 antibody.

As a conclusion, agonistic anti-CD38 antibodies, as represented by clones B08, C05, D06 and D07, increase IL-10 release from PBMCs in vitro. In addition, because IL-10 is involved in the general mechanism of inflammation and has been reported in numerous auto-immune diseases, agonistic anti-CD38 antibodies, as represented by clones B08, C05, D06 and D07, provide a new strategy to treat and/or prevent inflammatory and/or auto-immune diseases.

### Example 6: Neuroprotective Effect of B08 Anti-CD38 Antibody in an Experimental Autoimmune Encephalomyelitis (EAE) Mouse Model of Multiple Sclerosis (MS), an Auto-Immune, Neuro-Inflammatory Disease

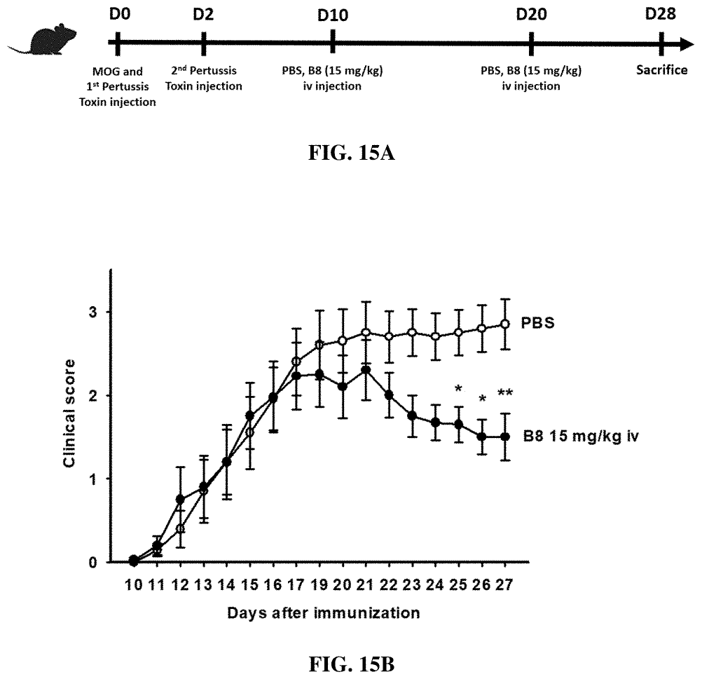

Multiple sclerosis (MS) is a progressive inflammatory and demyelinating disease of the human central nervous system (CNS) in which an important role for the immune system in the pathogenesis of the disease is suspected (Lassmann, 2008. J Neurol Sci. 274(1-2):45-7). EAE is an induced disease in mice that affects the central nervous system and constitutes a model for human MS. After a subcutaneous injection of an emulsion with a Complete Freund's Adjuvant (CFA) mixed with oligodendrocyte membrane proteins like myelin oligodendrocyte glycoprotein (MOG), an immune response starts in the periphery and, within 2 weeks, causes inflammation in the CNS. In the same time, we can observe, a progressive ascending paralysis (tail to hindlimb to forelimb). To determine whether B08 anti-CD38 antibody could have beneficial effect in this disease, we injected this antibody (15 mg/kg at symptom onset (10 days after MOG injection) and at day 20). We observed that B08 anti-CD38 antibody significantly reduced the clinical score compared with PBS-injected mice (FIG. 15).

As a conclusion, agonistic anti-CD38 antibodies (clone B08) ameliorate the clinical score in the EAE mouse model of the auto-immune, neuro-inflammatory disease MS when injected intravenously at symptom onset.

### Example 7: Anti-Inflammatory Properties of the B08 Agonistic Anti-CD38 Antibody in Vivo, in a Dextran Sulfate Sodium (DSS)-Induced Colitis Mouse Model

To demonstrate the effect of B08 agonistic anti-CD38 antibody in a purely inflammatory model, we decided to test its effects in the Dextran Sulfate Sodium (DSS)-induced colitis mouse model. Administration of DSS causes human ulcerative colitis-like pathologies due to its toxicity to colonic epithelial cells, which results in compromised mucosal barrier function. Clinical observations similar to human pathologies, such as weight loss, diarrhea and occult blood in stool, are commonly observed in the DSS model. Moreover, diminution of the colon's length is characteristic of this model. Importantly, studies suggest that DSS colitis mostly involves activation of lymphocytes, neutrophils and macrophages (Eichele and Kharbanda, 2017. World J Gastroenterol. 23(33):6016-29).

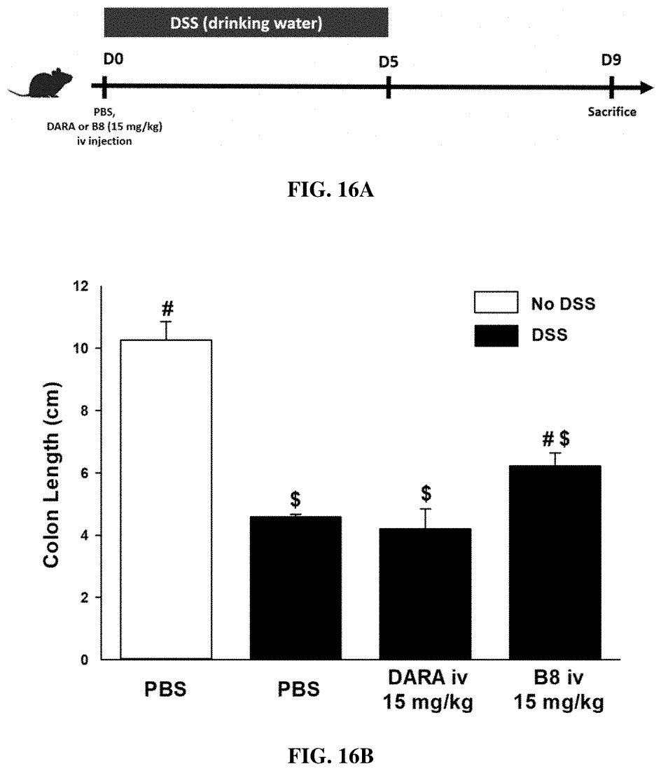

We observed that administration of B08 anti-DC38 antibody (15 mg/kg, iv) statistically significantly repressed reduction of the diminution of the colon's length (FIG. 16).

As a conclusion, agonistic anti-CD38 antibody (clone B08) increases colon length in the DSS mouse model of inflammatory bowel disease.

### Example 8: Use of CD38 Levels as Biomarkers for Therapeutic Use of sCD31 or Agonistic Anti-CD38 Antibodies

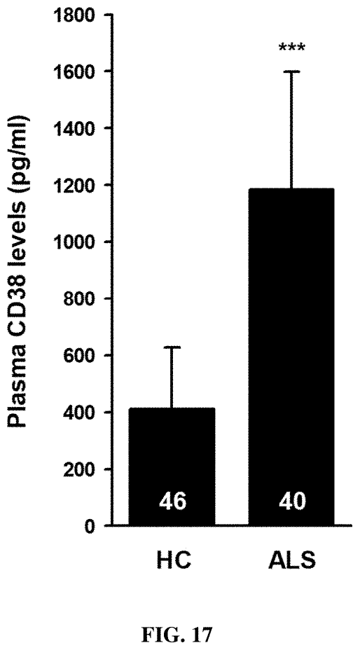

We observed that sCD31 and some anti-CD38 antibodies reproducing the binding of sCD31 to CD38 were neuroprotective. We therefore assessed whether measuring CD38 levels could be useful to identify patients that would benefit from such anti-CD38 treatment. We observed that plasma CD38 levels were strongly increased in plasma samples from ALS patients when compared to healthy controls (HC) (FIG. 17). This suggested that CD38 levels could be used as a marker to select patients to which administrate an anti-CD38 antibody as described herein.

### Example 9: Materials and Methods for Examples 1-7

1—In Vitro Experiments

1.1—Midbrain Cell Cultures

Animals were treated in accordance with the Guide for the Care and Use of Laboratory Animals (National Research Council, 1996), European Directive 86/609, and the guidelines of the local institutional animal care and use committee. Cultures were prepared from the ventral mesencephalon of gestational age 15.5 Wistar rat embryos (Janvier Breeding Center, Le Genest St Isle, France). Dissociated cells in suspension obtained by mechanical trituration of midbrain tissue pieces were seeded at a density of 1.2-1.5×10⁵ cells/cm² onto tissue culture supports precoated with 1 mg/mL polyethylenimine diluted in borate buffer pH 8.3 as described (Michel et al., 1997. J Neurochem. 69(4):1499-507). The cultures were then maintained in N5 medium supplemented with 5 mM glucose, 5% horse serum, and 0.5% fetal calf serum, except for the first 3 DIV, when the concentration of fetal calf serum was 2.5% to favor initial maturation of the cultures (Guerreiro et al., 2008. Mol Pharmacol. 74(4):980-9). They were fed daily by replacing 70% of the medium. Routinely, mesencephalic cultures were established on Nunc 24-well culture plates (Thermofischer Scientific, Rochester, N.Y.). Note that these cultures contain tyrosine hydroxylase (TH)⁺ neurons that were exclusively dopaminergic (Traver et al., 2006. Mol Pharmacol. 70(1):30-40). TH⁺ neurons represented approximately 1-2% of the total number of neuronal cells present in these cultures. The evaluation of the survival of DA neurons was performed by counting cells immunopositive for TH as described (Toulorge et al., 2011. Faseb J. 25(8):2563-73).

1.2—MPP⁺ Intoxication Model

Treatments with MPP were performed in cultures where the spontaneous death process was prevented by long-term exposure to depolarizing concentrations of K⁺ (30 mM), in the presence of the glutamate receptor antagonist MK801 (5 μM) to prevent unwanted excitotoxic insults as described previously (Douhou et al., 2001. J Neurochem. 78(1): 163-74). Treatments with MPP and potential neuroprotective molecules were carried out between 5 and 7 DIV.

1.3—Quantification of Neuronal Survival

The cultures were fixed for 12 min using 4% formaldehyde in Dulbecco's phosphate-buffered saline (PBS), then washed twice with PBS before an incubation step at 4° C. for 24 h with the following antibodies. A monoclonal anti-TH antibody diluted 1/5000 (ImmunoStar, Inc., Hudson, Wis.) was used to assess the survival of DA neurons. Antibody was diluted in PBS containing 0.2% Triton X-100. Detection of the primary antibodies was performed with an Alexa Fluor-488 conjugate of an anti-mouse IgG antibody.

1.4—Human PBMCs Cell Culture

3 healthy volunteers (Donor 1: woman, 23 years old; Donor 2: man, 29 years old; Donor 3: woman, 20 years old) served as blood donors. Venous blood (5 mL) was collected into heparinized tubes and immediately processed. Briefly, blood was diluted 1/1 in pre-warmed RPMI 1640 medium. PBMCs were isolated using gradient centrifugation in Ficoll solution (Sigma; 300×g, 30 min). Isolated PBMCs were re-suspended in 1 volume of pre-warmed RPMI 1640 medium and centrifuged again (300×g, 30 min). PBMCs were re-suspended in RPMI 1640 medium supplemented with 10% human serum (Sigma-Aldrich) at a concentration of 10⁷ cells per mL. Cells were seeded in 96 well microplate (100 μL per well). Test compounds (10 μg/ml), LPS (500 ng/mL) and interferon-γ 1B (100 ng/mL) were added to PBMCs culture immediately after plating. After 24 hours, cell culture supernatants were collected and frozen at −80° C. for further processing.

1.5—IL-10 ELISA Assay

IL-10 levels in cell culture supernatants were assayed using an ELISA human IL-10 kit (Ozyme®) according to the indications for use specified by the manufacturer.

1.6—Statistical Analysis

Simple comparisons between two groups were performed with Student's t test. Multiple comparisons against a single reference group were made by one-way analysis of variance followed by Dunnett's test when possible. When all pairwise comparisons were required, the Student-Newman-Keuls test was used. S.E.M. values were derived from at least three independent experiments.

2—In Vivo Experiments

2.1—Animals

Animals were treated in accordance with the Guide for the Care and Use of Laboratory Animals (National Research Council, 1996), with the European Directive 2010/63/EU, and the guidelines of the local institutional animal care and use committee. For all studies, 8 to 10 weeks-old male C57Bl/6 mice (Janvier, France) were used. They were maintained on a 12:12 h light/dark cycle with lights on at 8 a.m. The room temperature was kept at 20° C., with free access to standard diet and tap water.

2.2—6-OHDA and MPTP Mouse Models

a) Experimental Design

Two in vivo mouse models were used: the 6-OHDA mouse model and the MPTP mouse model. For the 6-OHDA mouse model, the intoxication protocol was based on the unilateral stereotaxic intrastriatal injection of 6-OHDA in the right striatum. Intracerebral or intravenous injections of treatments were made either the day before the surgical stereotaxic procedure, concomitantly with 6-OHDA during the surgical stereotaxic procedure, or two days after 6-OHDA injection. Animals were sacrificed 8 days after the surgical stereotaxic procedure.

For the MPTP mouse model, 4 injections of MPTP (20 mg/kg) were made. Intravenous injection of anti-CD38 clone HB7 antibody was done 2 days before MPTP injection. Mice were sacrificed 7 days after MPTP injections.

b) Surgical Stereotaxic Procedure

For unilateral 6-OHDA mouse model, animals were anesthetized using Chloral hydrate (400 mg/kg, i.p.) and placed into a stereotactic frame adapted to mice. The injection was performed using a Hamilton syringe at the following coordinates: AP: +0.85 cm, ML: ±2 cm, DV: −3.4 cm (corresponding to the atlas of Franklin and Paxinos, 1997). A total volume of 2.5 μL containing or not 5 μg of 6-OHDA diluted in PBS in the presence or the absence of test treatments was injected. The needle was left in place for 10 min after the injection before retraction.

c) Tissue Preparation

Mice were sacrificed by cervical dislocation, and beheaded to collect brain and blood samples. The brain was fixed in a paraformaldehyde solution (4%) during 1 day before being soaked in sucrose (30%) during 2 days and then froze at −80° C. for further analysis.

d) Brain Slicing, Immunofluorescence and Neurons Enumeration

Each brain was sliced using a freezing microtome at −30° C. The thickness of each slice was 20 μm. The slicing was done around the substantia nigra (80 slices from slice 51 to 63 according to Allen Mouse Brain).

Slices were then washed out in PBS and incubated with anti-Tyrosine Hydroxylase to stain dopaminergic neurons (Abcam®) during 2 days, at 4° C., with agitation. Slices were incubated with corresponding secondary antibody in the presence of DAPI (Sigma Aldrich®) to stain cell's nuclei during 2 hours at room temperature, and mounted on gelatin-coated slides.

The slices were imaged using a Nikon TE2000 U equipped with a Hamamatsu ORCE-ER camera or with a Zeiss Axio Vert.A1 equipped with an axiocam 503 mono camera. Image analysis was done using Image J software or Zen (Zeiss®).

2.3—IL-10 ELISA Assay

Blood samples collected in collection tube (Microvette® 500 EDTA-K3, SARSTEDT) were centrifuged at 3000 rotations per minute during 15 minutes. Supernatants (plasma) were collected and transferred into tubes and froze at −80° C. for further analysis. IL-10 levels in plasma samples were assayed using an ELISA IL-10 kit (BioLegend®) according to the indications for use specified by the manufacturer.

2.4—EAE Mouse Model

a) Experimental Design

Animals were anesthetized via inhalation with isoflurane (15 min). Mouse were immunized by subcutaneous injection of an emulsion containing 100 μg MOG peptide 35-55 in Complete Freund's Adjuvant and 500 μg HKMT (Heat-killed Mycobacterium tuberculosis) on day 0. Two hours later, animals received an intraperitoneal injection of 500 ng of pertussis toxin on day 0 and day 2. Test compounds were injected intravenously 10 and 20 days post-emulsion subcutaneous injection. Animals were sacrificed at day 28.

b) Clinical Score Evaluation

After immunization, animals were daily monitored for weight and clinical symptoms. Clinical scores were graded according to the following scale: 0: no clinical signs; 0.5: partially limp tail; 1: paralyzed tail, normal gait; 1.5: paralyzed tail, uncoordinated movement, hind limb paresis; 2: one hind limb paralyzed or does not respond to pinch; 2.5: one hind limb paralyzed and weakness of the other; 3: two hind limbs paralyzed; 3.5: two hind limbs paralyzed and weakness of forelimbs; 4: moribund state.

2.5—DSS Mouse Model

a) Experimental Design

Experimental colitis was induced in mice by giving drinking water ad libitum containing 3% (w/v) DSS for 5 days, while control mice received tap water only. Test compounds were intravenously administered (15 mg/kg) when DSS treatment began. Mice were sacrificed 9 days after the beginning of DSS treatment.

b) Statistical Analysis

The statistical analyses and figures were made using the software Sigma Plot. Different tests may be performed depending on the data: One-way or two-way ANOVA and post hoc tests, if the conditions of application are respected.

1-15. (canceled)

16. An isolated anti-human CD38 antibody or antigen-binding fragment thereof, wherein said antibody or antigen-binding fragment thereof competes with CD31 and induces lysosomal exocytosis in a cell, that is inhibited in the presence of vacuolin-1.

17. The isolated anti-human CD38 antibody or antigen-binding fragment thereof according to claim 16, wherein CD31 is human CD31.

18. The isolated anti-human CD38 antibody or antigen-binding fragment thereof according to claim 16, wherein said isolated anti-human CD38 antibody or antigen-binding fragment thereof is monoclonal.

19. The isolated anti-human CD38 antibody or antigen-binding fragment thereof according to claim 16, wherein said isolated anti-human CD38 antibody or antigen-binding fragment thereof is humanized.

20. The isolated anti-human CD38 antibody or antigen-binding fragment thereof according to claim 16, wherein:
a) the variable region of the heavy chain (HCVR) of said isolated anti-human CD38 antibody or antigen-binding fragment thereof comprises the three following complementary-determining regions (CDRs):
V_{H}-CDR1: GFTFSNX₁ (SEQ ID NO: 4),
V_{H}-CDR2: X₂GSSRX₃ (SEQ ID NO: 5), and
V_{H}-CDR3: X₄X₅X₆X₇X₈YX₉X₁₀X₁₁X₁₂GMDV (SEQ ID NO: 6); and
b) the variable region of the light chain (LCVR) of said isolated anti-human CD38 antibody or antigen-binding fragment thereof comprises the three following CDRs:
(SEQ ID NO: 21)
V_{L}-CDR1: AGTSSDVGGX₁₃X₁₄X₁₅VS,
(SEQ ID NO: 22)
V_{L}-CDR2: X₁₆DSX₁₇RPS, and
(SEQ ID NO: 23)
V_{L}-CDR3: STRVFGGGT;
wherein:
X₁ is Tyr (Y), Asn (N) or Ser (S);
X₂ is Ser (S) or Tyr (Y);
X₃ is Tyr (Y), Asp (D), Asn (N) or Ser (S);
X₄ is Ser (S) or is absent;
X₅ is Ser (S) or is absent;
X₆ is Ser (S) or Tyr (Y);
X₇ is Ser (S) or Asp (D);
X₈ is Tyr (Y), Ser (S), Asp (D) or Gly (G);
X₉ is Tyr (Y) or Gly (G);
X₁₀ is Ser (S), Tyr (Y) or Phe (F);
X₁₁ is Gly (G) or Asp (D);
X₁₂ is Asn (N), Tyr (Y) or Ser (S);
X₁₃ is Ser (S) or Asn (N);
X₁₄ is Ser (S) or Tyr (Y);
X₁₅ is Tyr (Y), or Ser (S);
X₁₆ is Tyr (Y), Ser (S) or Asp (D); and
X₁₇ is Tyr (Y) or Asn (N).

21. The isolated anti-human CD38 antibody or antigen-binding fragment thereof according to claim 16, wherein said anti-human CD38 antibody or antigen-binding fragment thereof comprises:
a V_{H}-CDR1 with SEQ ID NO: 7, a V_{H}-CDR2 with SEQ ID NO: 10, a V_{H}-CDR3 with SEQ ID NO: 15, a V_{L}-CDR1 with SEQ ID NO: 24, a V_{L}-CDR2 with SEQ ID NO: 29 and a V_{L}-CDR3 with SEQ ID NO: 23;
a V_{H}-CDR1 with SEQ ID NO: 8, a V_{H}-CDR2 with SEQ ID NO: 11, a V_{H}-CDR3 with SEQ ID NO: 16, a V_{L}-CDR1 with SEQ ID NO: 25, a V_{L}-CDR2 with SEQ ID NO: 30 and a V_{L}-CDR3 with SEQ ID NO: 23;
a V_{H}-CDR1 with SEQ ID NO: 9, a V_{H}-CDR2 with SEQ ID NO: 12, a V_{H}-CDR3 with SEQ ID NO: 17, a V_{L}-CDR1 with SEQ ID NO: 25, a V_{L}-CDR2 with SEQ ID NO: 30 and a V_{L}-CDR3 with SEQ ID NO: 23;
a V_{H}-CDR1 with SEQ ID NO: 7, a V_{H}-CDR2 with SEQ ID NO: 13, a V_{H}-CDR3 with SEQ ID NO: 19, a V_{L}-CDR1 with SEQ ID NO: 27, a V_{L}-CDR2 with SEQ ID NO: 32 and a V_{L}-CDR3 with SEQ ID NO: 23;
a V_{H}-CDR1 with SEQ ID NO: 9, a V_{H}-CDR2 with SEQ ID NO: 14, a V_{H}-CDR3 with SEQ ID NO: 20, a V_{L}-CDR1 with SEQ ID NO: 28, a V_{L}-CDR2 with SEQ ID NO: 30 and a V_{L}-CDR3 with SEQ ID NO: 23; or
a V_{H}-CDR1 with SEQ ID NO: 9, a V_{H}-CDR2 with SEQ ID NO: 14, a V_{H}-CDR3 with SEQ ID NO: 16, a V_{L}-CDR1 with SEQ ID NO: 28, a V_{L}-CDR2 with SEQ ID NO: 30 and a V_{L}-CDR3 with SEQ ID NO: 23.

22. The isolated anti-human CD38 antibody or antigen-binding fragment thereof according to claim 16, wherein said anti-human CD38 antibody or antigen-binding fragment thereof comprises:
a HCVR with SEQ ID NO: 33 and a LCVR with SEQ ID NO: 39;
a HCVR with SEQ ID NO: 34 and a LCVR with SEQ ID NO: 40;
a HCVR with SEQ ID NO: 35 and a LCVR with SEQ ID NO: 40;
a HCVR with SEQ ID NO: 36 and a LCVR with SEQ ID NO: 41;
a HCVR with SEQ ID NO: 37 and a LCVR with SEQ ID NO: 42; or
a HCVR with SEQ ID NO: 38 and a LCVR with SEQ ID NO: 42.

23. A nucleic acid encoding the isolated anti-human CD38 antibody or antigen-binding fragment thereof according to claim 16.

24. An expression vector comprising the nucleic acid according to claim 23.

25. A method of preventing and/or treating a disease in a subject in need thereof, comprising administering to said subject a compound which specifically competes with CD31 for CD38 binding, wherein said disease is selected from the group consisting of neurodegenerative diseases; neuroinflammatory diseases; inflammatory diseases; autoimmune diseases; metabolic diseases; ocular diseases; age-related diseases; and cancer and metastasis.

26. The method according to claim 25, wherein said disease is selected from the group consisting of amyotrophic lateral sclerosis; Parkinson's disease and related disorders; Alzheimer's disease and related disorders; Huntington disease; multiple sclerosis; rheumatoid arthritis; systemic lupus erythematosus; diabetes; obesity; non-alcoholic steatohepatitis; age-related macular degeneration; and glaucoma.

27. The method according to claim 25, wherein said compound is selected from the group consisting of a peptide, a chimeric peptide, an antibody, an antigen-binding fragment thereof, an antigen-binding antibody mimetic, an oligonucleotide and a small organic molecule; and wherein said compound:
induces tyrosine phosphorylation of discrete cytoplasmic substrates in a cell, that is inhibited in the presence of genistein; and/or
induces lysosomal exocytosis in a cell, that is inhibited in the presence of vacuolin-1.

28. The method according to claim 25, wherein said compound is an isolated anti-human CD38 antibody or antigen-binding fragment thereof, wherein said antibody or antigen-binding fragment thereof competes with CD31 and induces lysosomal exocytosis in a cell, that is inhibited in the presence of vacuolin-1.

29. The method according to claim 25, wherein said compound is:
a peptide comprising the Ig-like domains 1-3 of human CD31 or a variant thereof; or
a chimeric peptide comprising the Ig-like domains 1-3 of human CD31 or a variant thereof, fused to a Fc domain of an immunoglobulin, to human serum albumin or to transferrin.

30. A method of increasing the level of at least one anti-inflammatory cytokine in a subject in need thereof, comprising administering to said subject a compound which specifically competes with CD31 for CD38 binding.

31. The method according to claim 30, for increasing the level of said at least one anti-inflammatory cytokine in the blood of said subject.

32. The method according to claim 30, wherein said anti-inflammatory cytokine is interleukin-10 (IL-10).

33. The method according to claim 30, wherein said compound is selected from the group consisting of a peptide, a chimeric peptide, an antibody, an antigen-binding fragment thereof, an antigen-binding antibody mimetic, an oligonucleotide and a small organic molecule; and wherein said compound:
induces tyrosine phosphorylation of discrete cytoplasmic substrates in a cell, that is inhibited in the presence of genistein; and/or
induces lysosomal exocytosis in a cell, that is inhibited in the presence of vacuolin-1.

34. The method according to claim 30, wherein said compound is an isolated anti-human CD38 antibody or antigen-binding fragment thereof, wherein said antibody or antigen-binding fragment thereof competes with CD31 and induces lysosomal exocytosis in a cell, that is inhibited in the presence of vacuolin-1.

35. The method according to claim 30, wherein said compound is:
a peptide comprising the Ig-like domains 1-3 of human CD31 or a variant thereof; or
a chimeric peptide comprising the Ig-like domains 1-3 of human CD31 or a variant thereof, fused to a Fc domain of an immunoglobulin, to human serum albumin or to transferrin.
